SEQUENCE LISTING (188 sequences)

SEQ ID NO: 1 - protein, 98 aa, synthetic construct
  source 1..98: note = Zinc Finger QQR
EKLRNGSGDPGKKKQHACPECGKSFSQSSNLQKHQRTHTGEKPYKCPECGKSFSQSSNLQKHQRTHTGEKPYKCPECGKSFSRSDHLQRHQRTHQNKK

SEQ ID NO: 2 - protein, 92 aa, synthetic construct
  source 1..92: note = Lambda repressor DNA binding domain
STKKKPLTQEQLEDARRLKAIYEKKKNELGLSQESVADKMGMGQSGVGALFNGINALNAYNAALLAKILKVSVEEFSPSIAREIYEMYEAVS

SEQ ID NO: 3 - protein, 142 aa, synthetic construct
  source 1..142: note = Pot1 ssDNA binding domain
SDSFSLLSQITPHQRCSFYAQVIKTWYSDKNFTLYVTDYTENELFFPMSPYTSSSRWRGPFGRFSIRCILWDEHDFYCRNYIKEGDYVVMKNVRTKIDHLGYLECILHGDSAKRYNMSIEKVDSEEPELNEIKSRKRLYVQN

SEQ ID NO: 4 - protein, 206 aa, synthetic construct
  source 1..206: note = VirD2 domain
PDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPW

SEQ ID NO: 5 - protein, 114 aa, synthetic construct
  source 1..114: note = monomeric streptavidin domain
AEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVK

SEQ ID NO: 6 - protein, 33 aa, synthetic construct
  source 1..33: note = BRCA2-derived RAD51 binding peptide
PTLLGFHTASGKKVKIAKESLDKVKNLFDEKEQ

SEQ ID NO: 7 - protein, 69 aa, synthetic construct
  source 1..69: note = DSS1-derived RAD52 binding peptide
SEKKQPVDLGLLEEDDEFEEFPAEDWAGLDEDEDAHVWEDNWDDDNVEDDFSNQLRAELEKHGYKMETS

SEQ ID NO: 8 - protein, 288 aa, Homo sapiens
  source 1..288: note = PD-1 PROTEIN NP_005009.2 programmed cell death protein 1 precursor
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 9 - protein, 1719 aa, synthetic construct
  source 1..1719: note = dScCas9-FokI-ZFQ
MAVTPKKKRKVGGSGSEKLRNGSGDPGKKKQHACPECGKSFSQSSNLQKHQRTHTGEKPYKCPECGKSFSQSSNLQKHQRTHTGEKPYKCPECGKSFSRSDHLQRHQRTHQNKKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGA

SEQ ID NO: 10 - protein, 1730 aa, synthetic construct
  source 1..1730: note = dScCas9-FokI-Lam
MAVTAPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGSETPGTSESATPESTKKKPLTQEQLEDARRLKAIYEKKKNELGLSQESVADKMGMGQSGVGALFNGINALNAYNAALLAKILKVSVEEFSPSIAREIYEMYEAVSKRPAATKKAGQAKKKKGA

SEQ ID NO: 11 - protein, 1735 aa, synthetic construct
  source 1..1735: note = dScCas9-FokI-Strep
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGA

SEQ ID NO: 12 - protein, 900 aa, synthetic construct
  source 1..900: note = dScCas9-FokI-Vir
MAATPKKKRKVGGSGSPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKI

SEQ ID NO: 13 - protein, 1647 aa, synthetic construct
  source 1..1647: note = dScCas9-FokI-BRC2A2
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDKRPAATKKAGQAKKKKGA

SEQ ID NO: 14 - protein, 1683 aa, synthetic construct
  source 1..1683: note = dScCas9-FokI-DSS1
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDKRPAATKKAGQAKKKKGA

SEQ ID NO: 15 - protein, 1761 aa, synthetic construct
  source 1..1761: note = dScCas9-FokI-BRCA2-Strep
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKKRPAATKKAGQAKKKKGA

SEQ ID NO: 16 - protein, 1810 aa, synthetic construct
  source 1..1810: note = dScCas9-FokI-DSS1-Strep
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGSETPGTSESATPEAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKKRPAATKKAGQAKKKK

SEQ ID NO: 17 - protein, 1853 aa, synthetic construct
  source 1..1853: note = dScCas9-FokI-BRCA2-virD2
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWKRPAATKKAGQAKKKKGA

SEQ ID NO: 18 - protein, 1902 aa, synthetic construct
  source 1..1902: note = dScCas9-FokI-DSS1-virD2
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGSETPGTSESATPEPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWKRPAATKKAGQAKKKK

SEQ ID NO: 19 - protein, 1630 aa, synthetic construct
  source 1..1630: note = dScCasFok, 3NLS variant, Construct 13661
MAVTAPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDKRPAATKKAGQAKKKKPKKKRKVGA

SEQ ID NO: 20 - protein, 1663 aa, synthetic construct
  source 1..1663: note = dScCasFok-BRCA2, 3NLS variant, Construct 13656
MAVTAPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQKRPAATKKAGQAKKKKPKKKRKVGA

SEQ ID NO: 21 - protein, 1681 aa, synthetic construct
  source 1..1681: note = dScCasFok-DSS1, 3NLS variant, Construct 13659
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDKRPAATKKAGQAKKKK

SEQ ID NO: 22 - protein, 1717 aa, synthetic construct
  source 1..1717: note = dScCasFok-ZFQ, 3NLS variant, Construct 13648
MAVTPKKKRKVGGSGSEKLRNGSGDPGKKKQHACPECGKSFSQSSNLQKHQRTHTGEKPYKCPECGKSFSQSSNLQKHQRTHTGEKPYKCPECGKSFSRSDHLQRHQRTHQNKKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 23 - protein, 1733 aa, synthetic construct
  source 1..1733: note = dScCasFok-Strep, 1NLS variant, Construct 13665
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 24 - DNA, 16 bp, synthetic construct
  source 1..16: mol_type = other DNA; note = 3042-EMX1-T1-ddPCR-F1
gaaaccatgcccattc

SEQ ID NO: 25 - DNA, 17 bp, synthetic construct
  source 1..17: mol_type = other DNA; note = 3049-EMX1-T1-ddPCR-R2
agagaagggtggttttc

SEQ ID NO: 26 - DNA, 20 bp, synthetic construct
  source 1..20: mol_type = other DNA; note = 3088-EMX1-T1-TGEE3-reference probe
  misc_feature 1: note = modified with 56-FAM at 5'
  misc_feature 20: note = modified with Iowa Black Quencher at 3'
  misc_feature 9: note = Internal ZEN modification between nucleotides No's 9 and 10
ctctgtatggaaaagagcat

SEQ ID NO: 27 - DNA, 17 bp, synthetic construct
  source 1..17: mol_type = other DNA; note = 3089-EMX1-T1-TGEE3-drop off probe
  misc_feature 1: note = Modifyed with HEX at 5'
  misc_feature 17: note = Modifyed with Iowa Black Quencher at 3'
  misc_feature 8: note = LNA nucleotide
  misc_feature 9: note = LNA nucleotide
tcgtagagtcccatgtc

SEQ ID NO: 28 - DNA, 16 bp, synthetic construct
  source 1..16: mol_type = other DNA; note = 3175-EMX1-T1-TGEE4-drop off probe
  misc_feature 1: note = modified with HEX at 5'
  misc_feature 16: note = Modifyed with Iowa Black Quencher at 3'
  misc_feature 9: note = LNA nucleotide
  misc_feature 10: note = LNA nucleotide
atgtctgccggcttcc

SEQ ID NO: 29 - protein, 141 aa, synthetic construct
  source 1..141: note = RAD54 N-terminal peptide
RRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVH

SEQ ID NO: 30 - protein, 40 aa, synthetic construct
  source 1..40: note = RAD52 RAD52-recruitment peptide
PPAPPVTHSTPVTVSEPLLEKDFLAGVTQELIKTLEDNSE

SEQ ID NO: 31 - protein, 1763 aa, synthetic construct
  source 1..1763: note = ancestral dCas9-FokI-RAD54ntd (ancestral dCas9-FokI fused to a RAD54 N-terminal domain peptide)
MAVTPKKKRKVRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 32 - protein, 1662 aa, synthetic construct
  source 1..1662: note = ancestral dCas9-FokI-RAD52id (ancestral dCas9-FokI fused to a RAD52 peptide that interacts with RAD51)
MAVTPKKKRKVPPAPPVTHSTPVTVSEPLLEKDFLAGVTQELIKTLEDNSEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 33 - protein, 152 aa, synthetic construct
  source 1..152: note = MmeI nuclease domain
ALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQ

SEQ ID NO: 34 - protein, 121 aa, synthetic construct
  source 1..121: note = MnlI nuclease domain
KTTRNQGIVQYVRAYFPDECACCKNQYNIKDRSFTYRHSDRYYLEVHHVISFASDRTLDQIDNLVKVCPTCHRALSKNRADEQYQKELISEILINAPKAKEFCLNFTDENDCIQFIYDRLR

SEQ ID NO: 35 - protein, 182 aa, synthetic construct
  source 1..182: note = BfiI nuclease domain
NFFSLHPNVYATGRPKGLIGMLENVWVSNHTPGEGTLYLISGFSNYNGGVRFYETFTEHINQGGRVIAILGGSTSQRLSSRQVVEELLNRGVEVHIINRKRILHAKLYGTSNNLGESLVVSSGNFTGPGMSQNIEASLLLDNNTTQSMGFSWNDMISEMLNQNWHIHNMTNATDASPGWNLL

SEQ ID NO: 36 - protein, 1581 aa, synthetic construct
  source 1..1581: note = ancestral dCas9-MmeI
MAVTPKKKRKALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 37 - protein, 1550 aa, synthetic construct
  source 1..1550: note = ancestral dCas9-MnlI
MAVTPKKKRKKTTRNQGIVQYVRAYFPDECACCKNQYNIKDRSFTYRHSDRYYLEVHHVISFASDRTLDQIDNLVKVCPTCHRALSKNRADEQYQKELISEILINAPKAKEFCLNFTDENDCIQFIYDRLRSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 38 - protein, 1611 aa, synthetic construct
  source 1..1611: note = ancestral dCas9-BfiI
MAVTPKKKRKNFFSLHPNVYATGRPKGLIGMLENVWVSNHTPGEGTLYLISGFSNYNGGVRFYETFTEHINQGGRVIAILGGSTSQRLSSRQVVEELLNRGVEVHIINRKRILHAKLYGTSNNLGESLVVSSGNFTGPGMSQNIEASLLLDNNTTQSMGFSWNDMISEMLNQNWHIHNMTNATDASPGWNLLSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 39 - protein, 195 aa, Homo sapiens
  source 1..195: note = IGF1 (homo sapiens)
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 40 - protein, 390 aa, synthetic construct
  source 1..390: note = TGFB1
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 41 - protein, 1368 aa, synthetic construct [sequence omitted]

SEQ ID NO: 42 - protein, 1601 aa, synthetic construct
  source 1..1601: note = dScCas9-FokI, 1NLS
MAPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESMEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 43 - protein, 1625 aa, synthetic construct
  source 1..1625: note = dScCasFok, SV40+bipartite SV40 NLS (TG14280)
MAVTPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESMEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 44 - protein, 1631 aa, synthetic construct
  source 1..1631: note = dCasFok, 2NLS, 6His
MAVTPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESMEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 45 - protein, 1651 aa, synthetic construct
  source 1..1651: note = dCasFok, 2NLS, N-terminal BRCA2
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 46 - protein, 1657 aa, synthetic construct
  source 1..1657: note = dCasFok, 2NLS, N-terminal BRCA2, 6His
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 47 - protein, 1755 aa, synthetic construct
  source 1..1755: note = dCasFok, 2NLS, N-terminal Streptavidin
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 48 - protein, 1761 aa, synthetic construct
  source 1..1761: note = dCasFok, 2NLS, N-terminal Streptavidin, 6His
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 49 - protein, 1783 aa, synthetic construct
  source 1..1783: note = dCasFok, 2NLS, N-terminal Pot
MAVTPKKKRKVGGSGSSDSFSLLSQITPHQRCSFYAQVIKTWYSDKNFTLYVTDYTENELFFPMSPYTSSSRWRGPFGRFSIRCILWDEHDFYCRNYIKEGDYVVMKNVRTKIDHLGYLECILHGDSAKRYNMSIEKVDSEEPELNEIKSRKRLYVQNSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 50 - protein, 1789 aa, synthetic construct
  source 1..1789: note = dCasFok, 2NLS, N-terminal Pot, 6His
MAVTPKKKRKVGGSGSSDSFSLLSQITPHQRCSFYAQVIKTWYSDKNFTLYVTDYTENELFFPMSPYTSSSRWRGPFGRFSIRCILWDEHDFYCRNYIKEGDYVVMKNVRTKIDHLGYLECILHGDSAKRYNMSIEKVDSEEPELNEIKSRKRLYVQNSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 51 - protein, 1766 aa, synthetic construct
  source 1..1766: note = dCasFok, 1NLS, N-terminal Streptavidin, C-terminal BRCA2
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQ

SEQ ID NO: 52 - protein, 1794 aa, synthetic construct
  source 1..1794: note = dCasFok, 1NLS, N-terminal Pot, C-terminal BRCA2
MAVTPKKKRKVGGSGSSDSFSLLSQITPHQRCSFYAQVIKTWYSDKNFTLYVTDYTENELFFPMSPYTSSSRWRGPFGRFSIRCILWDEHDFYCRNYIKEGDYVVMKNVRTKIDHLGYLECILHGDSAKRYNMSIEKVDSEEPELNEIKSRKRLYVQNSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQ

SEQ ID NO: 53 - protein, 1662 aa, synthetic construct
  source 1..1662: note = dCasFok, 1NLS, ancestral RuvC+REC1/2, N- and C-terminal BRCA2
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQ

SEQ ID NO: 54 - protein, 1651 aa, synthetic construct
  source 1..1651: note = dCasFok, 2NLS, ancestral RuvC+REC1/2, N-terminal BRCA2
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 55 - protein, 1657 aa, synthetic construct
  source 1..1657: note = dCasFok, 2NLS, ancestral RuvC+REC1/2, N-terminal BRCA2, 6His
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 56 - protein, 3418 aa, Homo sapiens
  source 1..3418: note = BRCA2
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVANQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETSYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 57 - protein, 70 aa, Homo sapiens
  source 1..70: note = DSS1, homo sapiens
MSEKKQPVDLGLLEEDDEFEEFPAEDWAGLDEDEDAHVWEDNWDDDNVEDDFSNQLRAELEKHGYKMETS

SEQ ID NO: 58 - protein, 431 aa, Homo sapiens
  source 1..431: note = FOXP3 protein
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 59 - DNA, 695 bp, synthetic construct
  source 1..695: mol_type = other DNA; note = TCRA constant dCasFok guide 1
acgcgtaaaaaaaaagcaccgactcggtgccactttttcaagttgataacggactagccttattttaacttgctatttctagctctaaaacacagatatccagaaccctgaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgagagactctaaatccagttttagagctagaaatagcaagttaaaataaggctagtccgttatcaacttgaaaaagtggcaccgagtcggtgctttttttacgcgt

SEQ ID NO: 60 - DNA, 695 bp, synthetic construct
  source 1..695: mol_type = other DNA; note = TCRA constant dCasFok guide 2
acgcgtaaaaaaaaagcaccgactcggtgccactttttcaagttgataacggactagccttattttaacttgctatttctagctctaaaacgccgtgtaccagctgagagaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaagtctgtctgcctattcagttttagagctagaaatagcaagttaaaataaggctagtccgttatcaacttgaaaaagtggcaccgagtcggtgctttttttacgcgt

SEQ ID NO: 61 - DNA, 695 bp, synthetic construct
  source 1..695: mol_type = other DNA; note = TCRA constant dCasFok guide 3
acgcgtaaaaaaaaagcaccgactcggtgccactttttcaagttgataacggactagccttattttaacttgctatttctagctctaaaactaaatccagtgacaagtctgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttttgattctcaaacaaatgttttagagctagaaatagcaagttaaaataaggctagtccgttatcaacttgaaaaagtggcaccgagtcggtgctttttttacgcgt

SEQ ID NO: 62 - DNA, 695 bp, synthetic construct
  source 1..695: mol_type = other DNA; note = TCRA constant dCasFok guide 4
acgcgtaaaaaaaaagcaccgactcggtgccactttttcaagttgataacggactagccttattttaacttgctatttctagctctaaaacgtgacaagtctgtctgcctaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctcaaacaaatgtgtcacagttttagagctagaaatagcaagttaaaataaggctagtccgttatcaacttgaaaaagtggcaccgagtcggtgctttttttacgcgt

SEQ ID NO: 63 - DNA, 695 bp, synthetic construct
  source 1..695: mol_type = other DNA; note = TCRA constant dCasFok guide 5
acgcgtaaaaaaaaagcaccgactcggtgccactttttcaagttgataacggactagccttattttaacttgctatttctagctctaaaaccctattcaccgattttgattggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaaagtaaggattctgatgttttagagctagaaatagcaagttaaaataaggctagtccgttatcaacttgaaaaagtggcaccgagtcggtgctttttttacgcgt

SEQ ID NO: 64 - DNA, 695 bp, synthetic construct
  source 1..695: mol_type = other DNA; note = TCRA constant dCasFok guide 6
acgcgtaaaaaaaaagcaccgactcggtgccactttttcaagttgataacggactagccttattttaacttgctatttctagctctaaaacacaaatgtgtcacaaagtaaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatcacagacaaaactgtgcgttttagagctagaaatagcaagttaaaataaggctagtccgttatcaacttgaaaaagtggcaccgagtcggtgctttttttacgcgt

SEQ ID NO: 65 - DNA, 695 bp, synthetic construct
  source 1..695: mol_type = other DNA; note = TCRA constant dCasFok guide 7
acgcgtaaaaaaaaagcaccgactcggtgccactttttcaagttgataacggactagccttattttaacttgctatttctagctctaaaacagtaaggattctgatgtgtaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgctagacatgaggtctagttttagagctagaaatagcaagttaaaataaggctagtccgttatcaacttgaaaaagtggcaccgagtcggtgctttttttacgcgt

SEQ ID NO: 66 - DNA, 695 bp, synthetic construct
  source 1..695: mol_type = other DNA; note = TCRA constant dCasFok guide 8
acgcgtaaaaaaaaagcaccgactcggtgccactttttcaagttgataacggactagccttattttaacttgctatttctagctctaaaacacaaaactgtgctagacatgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagagcaacagtgctgtggcgttttagagctagaaatagcaagttaaaataaggctagtccgttatcaacttgaaaaagtggcaccgagtcggtgctttttttacgcgt

SEQ ID NO: 67 - DNA, 695 bp, synthetic construct
  source 1..695: mol_type = other DNA; note = TCRA constant dCasFok guide 9
acgcgtaaaaaaaaagcaccgactcggtgccactttttcaagttgataacggactagccttattttaacttgctatttctagctctaaaacgacatgaggtctatggacttggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggcctggagcaacaaatgttttagagctagaaatagcaagttaaaataaggctagtccgttatcaacttgaaaaagtggcaccgagtcggtgctttttttacgcgt

SEQ ID NO: 68 - DNA, 695 bp, synthetic construct
  source 1..695: mol_type = other DNA; note = TCRA constant dCasFok guide 10
acgcgtaaaaaaaaagcaccgactcggtgccactttttcaagttgataacggactagccttattttaacttgctatttctagctctaaaacgtgcaaacgccttcaacaacggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaccttcttccccagcccgttttagagctagaaatagcaagttaaaataaggctagtccgttatcaacttgaaaaagtggcaccgagtcggtgctttttttacgcgt

SEQ ID NO: 69 - DNA, 695 bp, synthetic construct
  source 1..695: mol_type = other DNA; note = TCRA constant dCasFok guide 11
acgcgtaaaaaaaaagcaccgactcggtgccactttttcaagttgataacggactagccttattttaacttgctatttctagctctaaaacacgccttcaacaacagcattggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcttccccagcccaggtaagttttagagctagaaatagcaagttaaaataaggctagtccgttatcaacttgaaaaagtggcaccgagtcggtgctttttttacgcgt

SEQ ID NO: 70 - DNA, 695 bp, synthetic construct
  source 1..695: mol_type = other DNA; note = TCRB constant dCasFok guide 1
acgcgtaaaaaaaaagcaccgactcggtgccactttttcaagttgataacggactagccttattttaacttgctatttctagctctaaaacgaggacctgaacaaggtgttggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgtttgagccatcagaaggttttagagctagaaatagcaagttaaaataaggctagtccgttatcaacttgaaaaagtggcaccgagtcggtgctttttttacgcgt

SEQ ID NO: 71 - DNA, 695 bp, synthetic construct
  source 1..695: mol_type = other DNA; note = TCRB constant dCasFok guide 2
acgcgtaaaaaaaaagcaccgactcggtgccactttttcaagttgataacggactagccttattttaacttgctatttctagctctaaaacggtcgctgtgtttgagccatggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccacacccaaaaggccacagttttagagctagaaatagcaagttaaaataaggctagtccgttatcaacttgaaaaagtggcaccgagtcggtgctttttttacgcgt

SEQ ID NO: 72 - DNA, 695 bp, synthetic construct
  source 1..695: mol_type = other DNA; note = TCRB constant dCasFok guide 3
acgcgtaaaaaaaaagcaccgactcggtgccactttttcaagttgataacggactagccttattttaacttgctatttctagctctaaaacgtggtcagggaagaagcctgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtggccttttgggtgtgggttttagagctagaaatagcaagttaaaataaggctagtccgttatcaacttgaaaaagtggcaccgagtcggtgctttttttacgcgt

SEQ ID NO: 73 - DNA, 695 bp, synthetic construct
  source 1..695: mol_type = other DNA; note = TCRB constant dCasFok guide 4
acgcgtaaaaaaaaagcaccgactcggtgccactttttcaagttgataacggactagccttattttaacttgctatttctagctctaaaaccacccaaaaggccacactggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttcttccctgaccacgtggttttagagctagaaatagcaagttaaaataaggctagtccgttatcaacttgaaaaagtggcaccgagtcggtgctttttttacgcgt

SEQ ID NO: 74 - DNA, 695 bp, synthetic construct
  source 1..695: mol_type = other DNA; note = TCRB constant dCasFok guide 5
acgcgtaaaaaaaaagcaccgactcggtgccactttttcaagttgataacggactagccttattttaacttgctatttctagctctaaaacccaaaaggccacactggtgtggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttccctgaccacgtggaggttttagagctagaaatagcaagttaaaataaggctagtccgttatcaacttgaaaaagtggcaccgagtcggtgctttttttacgcgt

SEQ ID NO: 75 - DNA, 695 bp, synthetic construct
  source 1..695: mol_type = other DNA; note = TCRB constant dCasFok guide 6
acgcgtaaaaaaaaagcaccgactcggtgccactttttcaagttgataacggactagccttattttaacttgctatttctagctctaaaacagctccacgtggtcagggaaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcacaccagtgtggccttttgttttagagctagaaatagcaagttaaaataaggctagtccgttatcaacttgaaaaagtggcaccgagtcggtgctttttttacgcgt

SEQ ID NO: 76 - DNA, 695 bp, synthetic construct
  source 1..695: mol_type = other DNA; note = TCRB constant dCasFok guide 7
acgcgtaaaaaaaaagcaccgactcggtgccactttttcaagttgataacggactagccttattttaacttgctatttctagctctaaaacactggtgtgcctggccacagggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgtggagctgagctggtgggttttagagctagaaatagcaagttaaaataaggctagtccgttatcaacttgaaaaagtggcaccgagtcggtgctttttttacgcgt

SEQ ID NO: 77 - DNA, 695 bp, synthetic construct
  source 1..695: mol_type = other DNA; note = TCRB constant dCasFok guide 8
acgcgtaaaaaaaaagcaccgactcggtgccactttttcaagttgataacggactagccttattttaacttgctatttctagctctaaaacgtgtgcctggccacaggcttggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagctgagctggtgggtgagttttagagctagaaatagcaagttaaaataaggctagtccgttatcaacttgaaaaagtggcaccgagtcggtgctttttttacgcgt

SEQ ID NO: 78 - DNA, 695 bp, synthetic construct
  source 1..695: mol_type = other DNA; note = TCRB constant dCasFok guide 9
acgcgtaaaaaaaaagcaccgactcggtgccactttttcaagttgataacggactagccttattttaacttgctatttctagctctaaaacggccacaggcttcttccctgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctggtgggtgaatgggaaggttttagagctagaaatagcaagttaaaataaggctagtccgttatcaacttgaaaaagtggcaccgagtcggtgctttttttacgcgt

SEQ ID NO: 79 - DNA, 695 bp, synthetic construct
  source 1..695: mol_type = other DNA; note = TCRB constant dCasFok guide 10
acgcgtaaaaaaaaagcaccgactcggtgccactttttcaagttgataacggactagccttattttaacttgctatttctagctctaaaacgccacaggcttcttccctgaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggtgggtgaatgggaagggttttagagctagaaatagcaagttaaaataaggctagtccgttatcaacttgaaaaagtggcaccgagtcggtgctttttttacgcgt

SEQ ID NO: 80 - DNA, 695 bp, synthetic construct
  source 1..695: mol_type = other DNA; note = TCRB constant dCasFok guide 11
acgcgtaaaaaaaaagcaccgactcggtgccactttttcaagttgataacggactagccttattttaacttgctatttctagctctaaaacgcttcttccctgaccacgtgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgaatgggaaggaggtgcagttttagagctagaaatagcaagttaaaataaggctagtccgttatcaacttgaaaaagtggcaccgagtcggtgctttttttacgcgt

SEQ ID NO: 81 - DNA, 695 bp, synthetic construct
  source 1..695: mol_type = other DNA; note = TCRB constant dCasFok guide 12
acgcgtaaaaaaaaagcaccgactcggtgccactttttcaagttgataacggactagccttattttaacttgctatttctagctctaaaacactgtgcacctccttcccatggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctccacgtggtcagggaaggttttagagctagaaatagcaagttaaaataaggctagtccgttatcaacttgaaaaagtggcaccgagtcggtgctttttttacgcgt

SEQ ID NO: 82 - DNA, 695 bp, synthetic construct
  source 1..695: mol_type = other DNA; note = TCRB constant dCasFok guide 13
acgcgtaaaaaaaaagcaccgactcggtgccactttttcaagttgataacggactagccttattttaacttgctatttctagctctaaaacggagctgagctggtgggtgaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagtggggtcagcacggacgttttagagctagaaatagcaagttaaaataaggctagtccgttatcaacttgaaaaagtggcaccgagtcggtgctttttttacgcgt

SEQ ID NO: 83 - DNA, 695 bp, synthetic construct
  source 1..695: mol_type = other DNA; note = TCRB constant dCasFok guide 14
acgcgtaaaaaaaaagcaccgactcggtgccactttttcaagttgataacggactagccttattttaacttgctatttctagctctaaaacgtgggtgaatgggaaggaggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcacggacccgcagcccctcgttttagagctagaaatagcaagttaaaataaggctagtccgttatcaacttgaaaaagtggcaccgagtcggtgctttttttacgcgt

SEQ ID NO: 84 - DNA, 695 bp, synthetic construct
  source 1..695: mol_type = other DNA; note = RNLS exon1 dCasFok guide 1
acgcgtaaaaaaaaagcaccgactcggtgccactttttcaagttgataacggactagccttattttaacttgctatttctagctctaaaaccctctcgccatggcgcaggtggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggatgacaggaagcttgtgttttagagctagaaatagcaagttaaaataaggctagtccgttatcaacttgaaaaagtggcaccgagtcggtgctttttttacgcgt

SEQ ID NO: 85 - DNA, 695 bp, synthetic construct
  source 1..695: mol_type = other DNA; note = RNLS exon1 dCasFok guide 2
acgcgtaaaaaaaaagcaccgactcggtgccactttttcaagttgataacggactagccttattttaacttgctatttctagctctaaaacctcgccatggcgcaggtgctggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatgacaggaagcttgtgcggttttagagctagaaatagcaagttaaaataaggctagtccgttatcaacttgaaaaagtggcaccgagtcggtgctttttttacgcgt

SEQ ID NO: 86 - DNA, 695 bp, synthetic construct
  source 1..695: mol_type = other DNA; note = RNLS exon1 dCasFok guide 3
acgcgtaaaaaaaaagcaccgactcggtgccactttttcaagttgataacggactagccttattttaacttgctatttctagctctaaaaccatggcgcaggtgctgatcgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaggaagcttgtgcgctgcggttttagagctagaaatagcaagttaaaataaggctagtccgttatcaacttgaaaaagtggcaccgagtcggtgctttttttacgcgt

SEQ ID NO: 87 - DNA, 695 bp, synthetic construct
  source 1..695: mol_type = other DNA; note = RNLS exon1 dCasFok guide 4
acgcgtaaaaaaaaagcaccgactcggtgccactttttcaagttgataacggactagccttattttaacttgctatttctagctctaaaacggcgcaggtgctgatcgtggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaagcttgtgcgctgcgctggttttagagctagaaatagcaagttaaaataaggctagtccgttatcaacttgaaaaagtggcaccgagtcggtgctttttttacgcgt

SEQ ID NO: 88 - DNA, 695 bp, synthetic construct
  source 1..695: mol_type = other DNA; note = RNLS exon1 dCasFok guide 5
acgcgtaaaaaaaaagcaccgactcggtgccactttttcaagttgataacggactagccttattttaacttgctatttctagctctaaaacctcagcagcgcagcgcacaaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcgcccacgatcagcacctgttttagagctagaaatagcaagttaaaataaggctagtccgttatcaacttgaaaaagtggcaccgagtcggtgctttttttacgcgt

SEQ ID NO: 89 - DNA, 695 bp, synthetic construct
  source 1..695: mol_type = other DNA; note = RNLS exon1 dCasFok guide 6
acgcgtaaaaaaaaagcaccgactcggtgccactttttcaagttgataacggactagccttattttaacttgctatttctagctctaaaaccgggatgacaggaagcttgtggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaggcagacgtccggtcccgttttagagctagaaatagcaagttaaaataaggctagtccgttatcaacttgaaaaagtggcaccgagtcggtgctttttttacgcgt

SEQ ID NO: 90 - DNA, 695 bp, synthetic construct
  source 1..695: mol_type = other DNA; note = RNLS exon1 dCasFok guide 7
acgcgtaaaaaaaaagcaccgactcggtgccactttttcaagttgataacggactagccttattttaacttgctatttctagctctaaaacaggaggcagacgtccggtccggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgggacaaggctgaggactgttttagagctagaaatagcaagttaaaataaggctagtccgttatcaacttgaaaaagtggcaccgagtcggtgctttttttacgcgt

SEQ ID NO: 91 - DNA, 695 bp, synthetic construct
  source 1..695: mol_type = other DNA; note = RNLS exon1 dCasFok guide 8
acgcgtaaaaaaaaagcaccgactcggtgccactttttcaagttgataacggactagccttattttaacttgctatttctagctctaaaacgtcccttgtaccttgctgtgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactcaggtgggttgtctaagttttagagctagaaatagcaagttaaaataaggctagtccgttatcaacttgaaaaagtggcaccgagtcggtgctttttttacgcgt

SEQ ID NO: 92 - DNA, 695 bp, synthetic construct
  source 1..695: mol_type = other DNA; note = IRE1alpha exon1 dCasFok guide 1
acgcgtaaaaaaaaagcaccgactcggtgccactttttcaagttgataacggactagccttattttaacttgctatttctagctctaaaacatgcaacagggagtaaggacggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcttctgccacttgccctggttttagagctagaaatagcaagttaaaataaggctagtccgttatcaacttgaaaaagtggcaccgagtcggtgctttttttacgcgt

SEQ ID NO: 93 - DNA, 695 bp, synthetic construct
  source 1..695: mol_type = other DNA; note = IRE1alpha exon1 dCasFok guide 2
acgcgtaaaaaaaaagcaccgactcggtgccactttttcaagttgataacggactagccttattttaacttgctatttctagctctaaaacggagtaaggactctcacccgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacttgccctgccggtttctgttttagagctagaaatagcaagttaaaataaggctagtccgttatcaacttgaaaaagtggcaccgagtcggtgctttttttacgcgt

SEQ ID NO: 94 - DNA, 695 bp, synthetic construct
  source 1..695: mol_type = other DNA; note = IRE1alpha exon1 dCasFok guide 3
acgcgtaaaaaaaaagcaccgactcggtgccactttttcaagttgataacggactagccttattttaacttgctatttctagctctaaaactcacccgagagctggcttctggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtttcttggtactgggagggttttagagctagaaatagcaagttaaaataaggctagtccgttatcaacttgaaaaagtggcaccgagtcggtgctttttttacgcgt

SEQ ID NO: 95 - DNA, 695 bp, synthetic construct
  source 1..695: mol_type = other DNA; note = IRE1alpha exon1 dCasFok guide 4
acgcgtaaaaaaaaagcaccgactcggtgccactttttcaagttgataacggactagccttattttaacttgctatttctagctctaaaacccgagagctggcttctgccaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttggtactgggaggctgcgttttagagctagaaatagcaagttaaaataaggctagtccgttatcaacttgaaaaagtggcaccgagtcggtgctttttttacgcgt

SEQ ID NO: 96 - DNA, 695 bp, synthetic construct
  source 1..695: mol_type = other DNA; note = IRE1alpha exon1 dCasFok guide 5
acgcgtaaaaaaaaagcaccgactcggtgccactttttcaagttgataacggactagccttattttaacttgctatttctagctctaaaacgcttctgccacttgccctgcggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaggctgcaggcttccttgttttagagctagaaatagcaagttaaaataaggctagtccgttatcaacttgaaaaagtggcaccgagtcggtgctttttttacgcgt

SEQ ID NO: 97 - DNA, 695 bp, synthetic construct
  source 1..695: mol_type = other DNA; note = IRE1alpha exon1 dCasFok guide 6
acgcgtaaaaaaaaagcaccgactcggtgccactttttcaagttgataacggactagccttattttaacttgctatttctagctctaaaactgcacagaaggaagcctgcaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaaccggcagggcaagtgggttttagagctagaaatagcaagttaaaataaggctagtccgttatcaacttgaaaaagtggcaccgagtcggtgctttttttacgcgt

SEQ ID NO: 98 - DNA, 695 bp, synthetic construct
  source 1..695: mol_type = other DNA; note = IRE1alpha exon1 dCasFok guide 7
acgcgtaaaaaaaaagcaccgactcggtgccactttttcaagttgataacggactagccttattttaacttgctatttctagctctaaaacgccggtttcttggtactgggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctgtgcaccgagacttccgttttagagctagaaatagcaagttaaaataaggctagtccgttatcaacttgaaaaagtggcaccgagtcggtgctttttttacgcgt

SEQ ID NO: 99 - DNA, 695 bp, synthetic construct
  source 1..695: mol_type = other DNA; note = IRE1alpha exon1 dCasFok guide 8
acgcgtaaaaaaaaagcaccgactcggtgccactttttcaagttgataacggactagccttattttaacttgctatttctagctctaaaacggtactgggaggctgcaggcggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagacttccttgagcaagtggttttagagctagaaatagcaagttaaaataaggctagtccgttatcaacttgaaaaagtggcaccgagtcggtgctttttttacgcgt

SEQ ID NO: 100 - DNA, 695 bp, synthetic construct
  source 1..695: mol_type = other DNA; note = IRE1alpha exon1 dCasFok guide 9
acgcgtaaaaaaaaagcaccgactcggtgccactttttcaagttgataacggactagccttattttaacttgctatttctagctctaaaacacagctccacttgctcaaggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaggaagcctgcagcctccgttttagagctagaaatagcaagttaaaataaggctagtccgttatcaacttgaaaaagtggcaccgagtcggtgctttttttacgcgt

SEQ ID NO: 101 - DNA, 695 bp, synthetic construct
  source 1..695: mol_type = other DNA; note = IRE1alpha exon1 dCasFok guide 10
acgcgtaaaaaaaaagcaccgactcggtgccactttttcaagttgataacggactagccttattttaacttgctatttctagctctaaaacagacttccttgagcaagtggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctcttggaagatcccgcatgttttagagctagaaatagcaagttaaaataaggctagtccgttatcaacttgaaaaagtggcaccgagtcggtgctttttttacgcgt

SEQ ID NO: 102 - DNA, 695 bp, synthetic construct
  source 1..695: mol_type = other DNA; note = IRE1alpha exon1 dCasFok guide 11
acgcgtaaaaaaaaagcaccgactcggtgccactttttcaagttgataacggactagccttattttaacttgctatttctagctctaaaacccttgagcaagtggagctgtggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagatcccgcatgggttcgttttagagctagaaatagcaagttaaaataaggctagtccgttatcaacttgaaaaagtggcaccgagtcggtgctttttttacgcgt

SEQ ID NO: 103 - DNA, 695 bp, synthetic construct
  source 1..695: mol_type = other DNA; note = IRE1alpha exon1 dCasFok guide 12
acgcgtaaaaaaaaagcaccgactcggtgccactttttcaagttgataacggactagccttattttaacttgctatttctagctctaaaactgagcaagtggagctgtgggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggatcccgcatgggttcgaggttttagagctagaaatagcaagttaaaataaggctagtccgttatcaacttgaaaaagtggcaccgagtcggtgctttttttacgcgt

SEQ ID NO: 104 - DNA, 695 bp, synthetic construct
  source 1..695: mol_type = other DNA; note = REST exon1 dCasFok guide 1
acgcgtaaaaaaaaagcaccgactcggtgccactttttcaagttgataacggactagccttattttaacttgctatttctagctctaaaacttatggccacccaggtaatgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaggagggctgtttaccaggttttagagctagaaatagcaagttaaaataaggctagtccgttatcaacttgaaaaagtggcaccgagtcggtgctttttttacgcgt

SEQ ID NO: 105 - DNA, 695 bp, synthetic construct
  source 1..695: mol_type = other DNA; note = REST exon1 dCasFok guide 2
acgcgtaaaaaaaaagcaccgactcggtgccactttttcaagttgataacggactagccttattttaacttgctatttctagctctaaaacaggtaatggggcagtcttctggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttaccagcagtggcaacatgttttagagctagaaatagcaagttaaaataaggctagtccgttatcaacttgaaaaagtggcaccgagtcggtgctttttttacgcgt

SEQ ID NO: 106 - DNA, 695 bp, synthetic construct
  source 1..695: mol_type = other DNA; note = REST exon1 dCasFok guide 3
acgcgtaaaaaaaaagcaccgactcggtgccactttttcaagttgataacggactagccttattttaacttgctatttctagctctaaaactttaccagcagtggcaacatggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacgacatgtatgacttgcgttttagagctagaaatagcaagttaaaataaggctagtccgttatcaacttgaaaaagtggcaccgagtcggtgctttttttacgcgt

SEQ ID NO: 107 - DNA, 695 bp, synthetic construct
  source 1..695: mol_type = other DNA; note = REST exon1 dCasFok guide 4
acgcgtaaaaaaaaagcaccgactcggtgccactttttcaagttgataacggactagccttattttaacttgctatttctagctctaaaactggaatggccctgcctaacgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgacctttccaaagctgaagttttagagctagaaatagcaagttaaaataaggctagtccgttatcaacttgaaaaagtggcaccgagtcggtgctttttttacgcgt

SEQ ID NO: 108 - DNA, 695 bp, synthetic construct
  source 1..695: mol_type = other DNA; note = REST exon1 dCasFok guide 5
acgcgtaaaaaaaaagcaccgactcggtgccactttttcaagttgataacggactagccttattttaacttgctatttctagctctaaaacgcctaacgacatgtatgactggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagctgaactggccgcacctgttttagagctagaaatagcaagttaaaataaggctagtccgttatcaacttgaaaaagtggcaccgagtcggtgctttttttacgcgt

SEQ ID NO: 109 - DNA, 695 bp, synthetic construct
  source 1..695: mol_type = other DNA; note = REST exon1 dCasFok guide 6
acgcgtaaaaaaaaagcaccgactcggtgccactttttcaagttgataacggactagccttattttaacttgctatttctagctctaaaacaataagctgaggtgcggccaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtcatgcaagtcatacatggttttagagctagaaatagcaagttaaaataaggctagtccgttatcaacttgaaaaagtggcaccgagtcggtgctttttttacgcgt

SEQ ID NO: 110 - DNA, 695 bp, synthetic construct
  source 1..695: mol_type = other DNA; note = REST exon1 dCasFok guide 7
acgcgtaaaaaaaaagcaccgactcggtgccactttttcaagttgataacggactagccttattttaacttgctatttctagctctaaaacgtatgacttgcatgacctttggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgcacctcagcttattatggttttagagctagaaatagcaagttaaaataaggctagtccgttatcaacttgaaaaagtggcaccgagtcggtgctttttttacgcgt

SEQ ID NO: 111 - DNA, 695 bp, synthetic construct
  source 1..695: mol_type = other DNA; note = REST exon1 dCasFok guide 8
acgcgtaaaaaaaaagcaccgactcggtgccactttttcaagttgataacggactagccttattttaacttgctatttctagctctaaaacgcatgacctttccaaagctgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcttattatgctggcaaatgttttagagctagaaatagcaagttaaaataaggctagtccgttatcaacttgaaaaagtggcaccgagtcggtgctttttttacgcgt

SEQ ID NO: 112 - DNA, 695 bp, synthetic construct
  source 1..695: mol_type = other DNA; note = REST exon1 dCasFok guide 9
acgcgtaaaaaaaaagcaccgactcggtgccactttttcaagttgataacggactagccttattttaacttgctatttctagctctaaaactccaaagctgaactggccgcggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctggcaaatgtggccttaagttttagagctagaaatagcaagttaaaataaggctagtccgttatcaacttgaaaaagtggcaccgagtcggtgctttttttacgcgt

SEQ ID NO: 113 - DNA, 695 bp, synthetic construct
  source 1..695: mol_type = other DNA; note = REST exon1 dCasFok guide 10
acgcgtaaaaaaaaagcaccgactcggtgccactttttcaagttgataacggactagccttattttaacttgctatttctagctctaaaaccacctcagcttattatgctgggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctggggaagtaaatggcaggttttagagctagaaatagcaagttaaaataaggctagtccgttatcaacttgaaaaagtggcaccgagtcggtgctttttttacgcgt

SEQ ID NO: 114 - DNA, 695 bp, synthetic construct
  source 1..695: mol_type = other DNA; note = REST exon1 dCasFok guide 11
acgcgtaaaaaaaaagcaccgactcggtgccactttttcaagttgataacggactagccttattttaacttgctatttctagctctaaaacctcagcttattatgctggcaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggaagtaaatggcagctggttttagagctagaaatagcaagttaaaataaggctagtccgttatcaacttgaaaaagtggcaccgagtcggtgctttttttacgcgt

SEQ ID NO: 115 - DNA, 695 bp, synthetic construct
  source 1..695: mol_type = other DNA; note = REST exon1 dCasFok guide 12
acgcgtaaaaaaaaagcaccgactcggtgccactttttcaagttgataacggactagccttattttaacttgctatttctagctctaaaaccagcttattatgctggcaaaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagtaaatggcagctgctgttttagagctagaaatagcaagttaaaataaggctagtccgttatcaacttgaaaaagtggcaccgagtcggtgctttttttacgcgt

SEQ ID NO: 116 - DNA, 695 bp, synthetic construct
  source 1..695: mol_type = other DNA; note = REST exon1 dCasFok guide 13
acgcgtaaaaaaaaagcaccgactcggtgccactttttcaagttgataacggactagccttattttaacttgctatttctagctctaaaacgcaaatgtggccttaactggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctgtgattacctggtcggttttagagctagaaatagcaagttaaaataaggctagtccgttatcaacttgaaaaagtggcaccgagtcggtgctttttttacgcgt

SEQ ID NO: 117 - DNA, 695 bp, synthetic construct
  source 1..695: mol_type = other DNA; note = REST exon1 dCasFok guide 14
acgcgtaaaaaaaaagcaccgactcggtgccactttttcaagttgataacggactagccttattttaacttgctatttctagctctaaaacaactggggaagtaaatggcaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcggtgaagaaagacaggttttagagctagaaatagcaagttaaaataaggctagtccgttatcaacttgaaaaagtggcaccgagtcggtgctttttttacgcgt

SEQ ID NO: 118 - protein, 283 aa, Homo sapiens
  source 1..283: note = PDX1
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 119 - protein, 214 aa, Homo sapiens
  source 1..214: note = NGN3
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 120 - protein, 353 aa, Homo sapiens
  source 1..353: note = MAFA
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 121 - protein, 370 aa, Homo sapiens
  source 1..370: note = WNT-1
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 122 - protein, 211 aa, Homo sapiens [sequence omitted]

SEQ ID NO: 123 - protein, 1375 aa, Streptococcus canis
  source 1..1375: note = ScCas9
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 124 - protein, 46 aa, synthetic construct
  source 1..46: note = E. coli Lac repressor fragment 1-46
MKPVTLYDVAEYAGVSYQTVSRVVNQASHVSAKTREKVEAAMAELN

SEQ ID NO: 125 - protein, 52 aa, synthetic construct
  source 1..52: note = HIV integrase fragment 219-270
MIQNFRVYYRDSRNPLWKGPAKLLWKGEGAVVIQDNSDIKVVPRRKAKIIRD

SEQ ID NO: 126 - protein, 64 aa, synthetic construct
  source 1..64: note = Residues 1 to 64 of the Sso7D DNA-binding protein of Sulfolobus solfataricus
MATVKFKYKGEEKEVDISKIKKVWRVGKMISFTYDEGGGKTGRGAVSEKDAPKELLQMLEKQKK

SEQ ID NO: 127 - protein, 64 aa, synthetic construct
  source 1..64: note = residues 1 to 64 of the Sto7D DNA-binding protein from Sulfolobus tokodaii
MVTVKFKYKGEEKEVDISKIKKVWRVGKMISFTYDDNGKTGRGAVSEKDAPKELLQMLEKSGKK

SEQ ID NO: 128 - protein, 73 aa, synthetic construct
  source 1..73: note = human HMG4. Comprising residues 2 to 79 of HMGB4
SDKPKRPLSAYMLWLNSARESIKRENPGIKVTEVAKRGGELWRAMKDKSEWEAKAAKAKDDYDRAVKEFEANG

SEQ ID NO: 129 - protein, 100 aa, synthetic construct
  source 1..100: note = Residues 1-100 of HMGN
MPKRKVSSAEGAAKEEPKRRSARLSAKPPAKVEAKPKKAAAKDKSSDKKVQTKGKRGAKGKQAEVANQETKEDLPAENGETKTEESPASDEAGEKEAKSD

SEQ ID NO: 130 - protein, 83 aa, synthetic construct
  source 1..83: note = Residues 1-84 of HMGB1
GKGDPKKPRGKMSSYAFFVQTCREEHKKKHPDASVNFSEFSKKCSERWKTMSAKEKGKFEDMAKADKARYEREMKTYIPPKGE

SEQ ID NO: 131 - protein, 79 aa, synthetic construct
  source 1..79: note = Residues 1-79 of HMGB3
MAKGDPKKPKGKMSAYAFFVQTCREEHKKKNPEVPVNFAEFSKKCSERWKTMSGKEKSKFDEMAKADKVRYDREMKDYG

SEQ ID NO: 132 - protein, 74 aa, synthetic construct
  source 1..74: note = StkC domain comprise residues 232-305 of Arabidopsis MBD7 methyl-CpG-binding domain
SVSMVHSERLPLLMNRNGIRFQSEVIDPNPPKKVKWVLTGSGGNMFTANVRGSNVSSLVKHTWSEAFVSLIEDR

SEQ ID NO: 133 - protein, 194 aa, Flavobacterium Okeanokoites
  source 1..194: note = FokI
VKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 134 - protein, 194 aa, synthetic construct
  source 1..194: note = Consensus FokI mutant
VKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINF

SEQ ID NO: 135 - protein, 194 aa, synthetic construct
  source 1..194: note = Enhanced FokI nuclease domain
VKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 136 - DNA, 11080 bp, Homo sapiens
  source 1..11080: mol_type = unassigned DNA; note = human MPO gene
gacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccaggtaacagttcccaaggtgggagaagatggtgtgttggggtggttgtgttccagagaccccttttctcagagcaggccttcctagctctggggcctgatagggggttggggcccattcctgactttgtgatccctgctctgggcagctgtcctgggggaggtggacacctcgttggtgctgagctccatggaggaggccaagcagctggtggacaaggcctacaaggagcggcgggaaaggtggggcacgaggcactgcccagctctgggcaaggatgtcccaggcctttcagagaagcagcaggcagcagggagcttgaaggtgggagagggggactatggggacccatgtggactccctttctgtctgtctgtccctgtgtttctggatgggagaacagcttaaaagccatccttcccaaagccttgcctctgtctggacacccaggtccatatcctttttataaaaggaagccttcctcccacccgtgggcctgcctgcatggtcctgcatgtctgcccctgggctggggctctgctgggtgggtctgtatcccttctctggccctcactcctcctttctccaagcagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactggtactgttgcccatcacaccccaggtccctgctccactagtgactcctcctagggaccccagccgtctctcagtgatcccctcaacttcttcctccagggagtctcaggagtctccagggctcctcagcctcaggttgcctgggataggaagtgaggcggctcagctcccccattgttctttcccccggcagatgtgctgacgcccgcccagctgaatgtgttgtccaagtcaagcggctgcgcctaccaggacgtgggggtgacttgcccggagcaggacaaataccgcaccatcaccgggatgtgcaacaacaggtgcggctggctgggggtggctgcaggaaccgggctcagagaggcgtcccggacgccacaagcctcccggtgtcagcgccctgtcctcccctgcagacgcagccccacgctgggggcctccaaccgtgcctttgtgcgctggctgccggcggagtatgaggacggcttctctcttccctacggctggacgcccggggtcaagcgcaacggcttcccggtggctctggtgagcgccggcgggcagagggggcgaggcccggccacgcggtgcgcggacccaggcgccagctgatctccgtgtcccgcaggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaaggtggccctgcttcccgcccactgcctgggttgggagaggggagattgtttctggaaggggccatctcccttctgtgcccaggtttcctttcccagacgctgaggaaggctggccctgcctcccttgtgtccacagcactggctgctcagctatgacctgtctccttcctgcgcctgggctcagccagctggcagccagggccgtccatttgctcactgcctctggcaggccaagggagagagggttgttcctccttggctgacaggagtcctgttggtggaatcactggctctttgttaggttgggagagggttctgggaggagaccttctcagtgccccccaagcctccatgctcagtcctgtttccgccccactgagagggcagccccaacccccatcacacagaaagagagagcctgggggtctggtgggtgccgtgggacagggctgggagtgggcagggtggtcgaggcactgctgagtagggaaggaggaggagagaaaggagagagcgcctgagagagggagagacagacaggagaatgtcagggccagagggagagcaggcacagcagagagaaggggagagagacgggcgacgctttagtgaggagggatccgaggcctggcggtgcccagacccaggggttgcccaggttcccagttcagtgttctgctcattaaccctgcacctcagaggctgttgctgatggcgccctaggcagcgatccctttcgggcctccggaggcctctctgcaggttgagggtaccagaggtcctgagggcagggggagatccagttctgcctgggcaccttccttgccctcggtgagccagtctagcctctctctgtgcctcagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggtcagctttggggtggggaccagaggtggcataggagatgttccctgttggagccacagtgagtctgtttgtgagcagcttgtgggtttgtacttagagagactgtcctcaccagccattactatcaacttcatgatattaatccagttgccttggtaacaagtcagatggagccagaaaagcagaaaagcaaacaacctctccaaccctaagatggagacccaggcatggctgaggagcctctgtccatcagtccattgctttctctgtcccttttttcccttctggactctggaagacaagagagtcaaatccctcaggggcagggaactaaaggacagagaacgctgtggtgccctcaccctctagagcgacactatccaatgtggtagccactagctgcatgtggctattttaattttagttattttaaattaaataaacccagtttctcagcggtgctaaccacatttcaaatgctcaattgccttacagtctagtggctccccctattgggtggcacagatgtaaaatattttcatcatctcggaaggttgagttggaccacactgctccaaaatatcccctcctgcgcatcttaattacccataataagagatgatgggattttaaatgtaaacaattggcagttttggagacacaattggggcagtgatggcagccatgaccagacattttccagtgttttttttttttctaagacggaatctcactctgtcacccaggcttgagtacagtggcacagtctcggctcactgcaacctctgccaactgggtttaagtgattcttctgcctcagcctcctgagtagctgggactacaggcgcctgccaccacacttggctaattttttgtatttttagtagagatggtgttttaccatgttggccaggctggtctcaaactcctgacctcatgatctgcccaccttaacctcccaaagtgctgggattacaggtgtgagccaccgcacgcagccattttccagtgtttttaaatgtaaacccaatagtggacctcaggccctttcctctggcctccctctgcctcctcctgatggttcctgctggtctctccctggctgggaccctgggaggaggatccaggtcggactttctgcaagagatgcccgacctcttctgggctagttaggcaagggcagccctccttagccagactcagctgcccacagcctggctttgatgagccccatgtcccaagcctgggtcattatgacctctagctgttcatggatggagatgctgctctcagagaatttcctttccaccagcttctagagctggcaaggactgaccctagtgtcctgaccctagccctctggcccctgcagagaagggttctgggaggactaggggaaccaggggggatctgcttagttttcttgaacagtctcaggtgtggactccacatatctcatgtcctagccctctcccctggggcaggctgcaccattggcatgtagtaaagggcagggctgacaactctgttcaaatggtagtggctttctggagtcctgtgttgagaaggattctgaggttgcatccaattttagcaggaaagagtgctgtgatcgttagctgcatcagacatgggcatggatgtaggctgtgccaacacgcatgcttcttcccagacactccttggaatcaagagaatcaatgaaccaagcagtgttcataagtctcatactttgtaccaggcactctgttaagccccatgtatccatatgtgtgctaggagggagggtaaaatataaaaaggcacaagccttggtcctgtgctcaaggagctcccagtttcaggtacaggaaagtggcatgtcatgtgttggaagtgatacctattgaatgagcatccccaaccagcagcggccctgagctgaggcagaagctatcattgtggactggggcatcagggaaggcctcctagaggagtgtaaattggggatagtttgaaaagatggatggggaggaggggtgagtaggaggcatttcaggccttggctggggagggggtttcagtggagcaaatcttttctgggatggaggccttaagaatgacagtggcttttgcctccccaaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccaggtaggccgtcttgaacaccgggcacacgggatccagatgtgtccctgcaacatcctagctgtgtgaccttgggcaagttcctaacgctcctgagccttgccttcttcatccatgaggctgtaaggattataatacatatatgaaaacacctttcagatagtaagtgcttgacaaagcctccttcccttccctctccttcttcgcaggtttgctccctgcagtcttctttcctgtgggtctgttattggtgttgggagggttgaatgtttggtttagcgcccaagtagctgttcagtaaattcttcttccctccatctcactgtcactcttagctcctttacccactcacctcttctttttaaaattaaaaaaattatagggctgggcgcggtggctcatgcctgtaatcccagcactttgggaggcggaggcgggtggatcacgaggtcaggagatcaagaccatgctggctaacacggtgaaaccccatctctactaaaaatacaaaaattagccgggcgtggtggcgggcacctgtagtcccagctacttgggaggctgaggcaggagaatggcgtgaacctgggaggcagagcttgcagtgagctgagatggcgctgctgcactccagcctgggtgacagagcgagactccatctcaaaataaataaataaataaaataaaatttaaaaattattattattattttatttttattttttgatacagggtcttaccctgttgcccaggctggagtgcagtggtgcgatcttggctcactgcaacctccacctttcaggctcaagccattctcccatctcagccttctgagtagctgggatttcaagcacgggccaccacgcccggctaatttttgtattttttgtagagacggggtttcgccacgttacccagtgtggtctcaaactcctgggctcaagtaatccgccctcctcgacctcccaaatccctgggattacaggcacgagtcactgtgttcagcccactcacctcttctttttggctctgtcataatccacccaatgtccttagcctcatttacccctggggcaatgttcctggttctgtctctcaacatctggtgcttcattgcccccaccctctccctcagaggcagaggcagacatacacacactcacacacacacacacacacacacacacacacacacactcctagattctggaggagagagttttcggtgctaagggtgagtttttgatttgcactaagaactcaagtgaaattcatttacactccaaaataacttctagactctagaatgtagagtgagactattccatgtcttcacttttcacagggaaaatcatgccagaatggcccttcgttccttcctgcctccctgctttccacaaacctaaactgagccttactgtgggcaggcacttggtgggcactgtgaagaatttcaacaacttcaatttcccttgctctcaaggactttataagatagttgcagaaatgagacattaataacttgaggacaaggaagttatcacaatgactaaaaaagtgatttgctatttagtcctctaaggcagagggcaatataattatccttaatgtacatttgaggaactcaggctcagagaggttaataattggtccaaggtcacccagccagcaagcagtagaactctatttatgcctctcagtgccactctgttaggcccaggtccggtattgtgggaggctattccctgaccatagagggatggaaggggtccccgaagccaagagcaggcagagactctggccctcctgtgctgccaggcggcatttgctgtggaggagtgggtggagaggccattccaaatgacttgtctttagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtaagcaggacctaggccaggagcgagtgggctgacagctaaaggtggggtagggatcaccttggctctagggagctagggtggggagcagtctgctttcctcccatcttggagatctggtctgactctctagcctcagttcctctgactctgcttccatatctgataataaacagtggggtggggatggccctcagagggaactggctacttcctatgtgttgtggctcgctgtgacccaggaagccagggcctgggagcatcccaatcttgcaagtctgaatggcataatctcctgagacctatatgtccctggggtcatgtagtgaccggtgaggacctgctcactggggccaccaccttggcaccaggcactgcttgtgcccagccctcttctcgaatcctcctgaccctacctggacttgtccctgactccaatctgagctctgatactgagccagatacttcccctgacctggctcctctggtccccaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggtgaggggctgcaggagtctcccctgatgctcacctcccctgagctgcctcctaggtagcccatcaccccccttcccactctagggtccctgccctctccaaagcatattagaggcactttgctctcaggaagtagcctgaggccaggcccctgccttttcccggacccacctgataaatttccagctcctctcaacctcctttgcatgctgtaattctcatattaggggcatcgtcatcataatgataacccacagtctgaaaatctgtctctgtcatcattccattttgcttcattgtacgttaactctgggggaagagattattattgccttcattttactgatgaggaaactgaggcttgatgagattcagaacctggccaggttctctagccaagtcagtggcaaagctgaaatttgaagccagattagcctaacctcaaagcctgtgcctatgttatggggtagcccatgtggctgaggcttctagacacatttcacctctcatgggtcagagacagacacatcgggctgctctggagggggacaaggagatgtccctattcttggacccagcaggaactgtggcagtttagggtttgccttatggtaggagggcaagagaaagacagagagggtcccagagaggcccagttgtgccccttggtctaagccaaaggtacagggttccttgaggtgaggtccttccctccctctatgaccttggttgtgttgggaaggtgggttttctgggctgctggcgatatgtgaatcttccctgctgtctccagtgacctccccaccttgaagcagagagacctgcccatagccacctgtcccctcccccatgcaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggtgaggaggggcaggcgtcgtgggccgctgggtggctgtgggcccatccttgactctcttggagcccaaatttcctcctgtcagttgaaggactggagggagtcagtaattttccagtgtgttccaggaatcctccagatgccctggagtgcccctaatgtgccctgaacctgttgggggtgcagcaaggaaagacctggacccctggttgtaaagggtggaagatagaaaagtgccacccttagctacttcttcccatgggtttcactgtgccaccatgtgggtgtcagtcaccagtgggcagtcccctcccagctttcccacagctccccttagtgtcatttcctcgtggtcctgatgtcttcatgtctagttcttgcttagcctgtactgcacctttgggaaaattagaaaaaggtgcccctctctgggtgggcgaattataaaccacccccaagattctcttccactcctgtggatgcagctcctggccagggcacagggcttcaggagtgtgtgtgggcccgggtccccctccagcatggtgccctggaggccgccgggctaggggtaagggaaaggccactgggcagctgtgctttactctgcacgatgagtggagcatcacttgtgtgaaagcccctgggctgcccaagggcctggggccctcctgtgcttccaggtggcatttgttgtggctttgttatatcctgggagcagcacaagcccatcgatgccctgccagcccagaatatccttgggcacagtgtccatgggtgttccccatgcaggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcc

SEQ ID NO: 137 - protein, 745 aa, Homo sapiens
  source 1..745: note = MPO protein Accession Number: NP_000241
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 138 - DNA, 10629 bp, Mus musculus
  source 1..10629: mol_type = genomic DNA; note = MPO chromosome 11, GRCm38.p4 C57BL/6J DNA SEQ. ACCESSION: NC_000077
ataacttctcagctttagctccctgaattttggttctttcaaaggatttgggacactcagtttaaccaacactttgaggggctggttgtttgtgctctccgggagaagctctgatccattcctttgtccacccctccttcttcatagacccctttctgtgcacccctaaccccagtgggggaggagagtctagtcttgggataggtccatctctttttaggtcagcccgcccccagcttttacaccccaggcataaaaacccgttgtcagtgagaggagttgacagtgtcaggtaagtggtgaagttgagctcttggggactggggactatagctggagggggtcttgggttgtacacctcttcatagaccaggcccactagggtagggctgtgtataaaggggagaggagggggcttgggaggaggagaattgtaaaacctctcttttttcaaaagctctggatacttcctgagctgaggtttttgttttttgtcttgttttgttttgttttgttttgttttgttttgttttgttttgttttgttttgtttttttaaaggtttatttatttattatatgtaagtacactgcagctgtccttagatactccagaagagagcatcagattttgttacggatggttgtgagccaccatgtggttgctgggatttgaactcgggacctttggaagagcagtcggcgctcttaaccactgagcaatctccccagcccccttgttttttgtcttgcagagatgaagctactcttggccttggcaggcctcctggcccctctggccatgcttcagacctccaatggtgccaccccaggtgatagtgccaaaaggagggtggactaagggattgggggtagggggtggggattctcagcccagcatctccccttcccctggataggcttcctttgctttggggcctgatgtagggctttggggccagccctgaccttatgatttatgctctgggcagctctcctgggggaagtggagaattcagttgtgctgagctgtatggaagaggccaagcagctagtggacagagcctacaaggagcggagagaaaggtgagggtgccaggccctgtgtggccctgggcaaaactgcttcaagtcttctggagaaagggcacacagtggggggaggggggttgtagaaaggatgagggcctacaggtgaaatcgtacctggtctgcctctacttgagtttctggctgtcagaacatcttcagtggccttgctttctgtctggacatccaggtccatgtccttttgacaaaagaaatgcactgctcctccgtggttctctccagagttctttctgcctgctcaccgcaggctggcctttgctgagtgtctctatcccccttctctggccctcccttgtctaaacagcatcaagcggagcctccaaagcggctctgccagccccacggagctcctgttttacttcaagcagccggtggcgggcaccagaacagctgtgagggccgctgattatctacatgtggccctagacctgctgaagaggaagctgcagcccctgtggccaaggcctttcaatgttacaggtactctaactccttttgtgctaccacctccaccccaagcaacttctgtctccaagcctccattcaaggcagtggttcttaacctgtgggtcgctaccccttcgggtacgtcaagtgaccctctcacatgggtccacctaagaccataggaaaacacaggtcttaacattatgattcatctcagtagcaaaattacagttatgaagtagcaagtaattttatagttctgttcaccccatgagaaactggcttaaggggtctctcagcattagcaaacttgagaaccactggtctaaggagtctcggggcctctgagctggcccacccccctcctcaccctcactgggctactcagccccaggttgcctagcataggaaatgaggtgggggaaggctcggatctaagatcatggctcactcttgcagatgtgttgacacctgctcagctgaatctgttgtccgtgtcaagtggctgtgcctatcaggacgtgagggtgacatgcccaccgaatgacaagtatcgcaccatcactggacactgcaacaacaggtgtgggcccagggcagttcagggttgtgtggtgtagggggtctaggtcttgcctgtggcgacccattgggccagagctctgtgcttccctctagacgaagccccactctgggggcctccaaccgtgcctttgtacgctggttgcctgcagagtatgaagatggcgtctccatgccctttggctggacgcctggagtcaatcgcaatggcttcaaggtgcccctggtgagtgtccagtagagggaggtgagcccagctaagcttatgtgcaggacctaggcatccaatgactcctgttgttgctgcaggctcgccaggtctccaatgccatcgtgcgcttccccaacgatcagctgaccaaggaccaggagcgtgcactcatgttcatgcagtggggacagtttctggatcatgatatcaccttgactccagagccagctacccggttctccttcttcactggcctcaactgcgagaccagctgcctgcagcagccaccctgcttccccctcaaggtgctgcctccttccatccatcagggtctgtgtctgaagagagggaagatggtttacattagtgacggcgccccttctgagtcctaggtcctctctagcccctgagcaagactggtcctcgctcctgggcccagagtcaatacttggctccctcagcagagccagctgtcaactcaaggaacctgatatgcaccctgatatgcaccctgaacttagcatcgctctgtgtggtggggtgactggttctttgtgaggctgggagaattctgggatgaggatctgacttgtcttcttcatactcccaatgtacaggcactggcctgttctcactctctctcccctatgggcgctcccctaaccctgcttcacagtgtgtgtgtgtgtgtgtgtgtgtgtgtaaccctgcttcacagtgtgtgtgtgtgtgtgtgtgtgtgtgtgtgtgtgtgtgtgtgtgtgtgtgtgtgtgtgtttgggttgccccatagtcagggtcagggttgaagcaccttctgtgtaggggggaaagtgtggagaaaaagaaaggcagagattaaggagataaagggccaggggtctgacagggccagggtgacagaacagagaggaaaaggcagacacaggcaagaagaggattaagaggcatagtgaagaggggtccaagtggccttgcccaaccccagaaggctctctccattccagttcagtgaattctttgtcacccttaaacctcagtctctttgctgactgagccccagtatttctatgaaaccttgagagcttccctgcagctcgagggggccagagttctcttctgcagggtgtaagttacaaatgcccgctctcccgacccacaaaagccagcctagcttctctctgtgctgtagatcccacccaatgaccctcgaatcaagaaccaaaaggactgcatccccttcttccgctcctgcccggcatgcaccaggaacaacatcaccattcgcaaccagatcaacgcgctcacttccttcgtggacgccagcggggtgtacggcagcgaggaccccctagccagaaagctgcgcaacctcaccaaccagctggggctgctggctatcaatacacgcttccaagacaatggcagggccctgatgccctttgacagcctgcacgatgacccctgcctcctcaccaaccgctccgcccgcattccttgttttctggcaggtcagcccggggactgaactagagtggcatagagctgtttccagcttgtgagaacacagcctgtatgtgagcaacatgcgggcttgtgataagagatgctggtctcaacaacttattagtctagttgccttggtaacgagttggatggagacagaaaggaaaataacagcaaaaatcaaaccacataagcatacaaaaaaaaaatcctccatccatcaactgaagatggagtcacagggctgctaaggaccctctattattgatccagctttctctgtcttctccctcctgtggactgtggaaaaagagagagccaagtctatgtccacaagggacagagaactaaaagtgacagagaggaatatggcatcctcaccttttagaaccatcctgtccaatctggagctagtagccacatgtggttatttcaatttaaattctttgtagcagggagtactggctcacacctttaatcccagtctttgggaggtagaggcatgaggctcttagtgagaattcaaggccagcctggtctacagagtgagttctaggacagccagggctatagagtgatagtgagaccctgtcttgaaagaagaagaagaagaagaagaagaagaagaagaagaagaagaagaagaagaagaagaagaagaagaagaagaagaagaagaagaagaagaagaagaaaacaaccaataaatacataaataattttaaattaaattaaaagtttctgcagtaactgcattttaagggctcagtagtccatgggtgaagaagtggttgttttcttcacctcagaaaattctagtagattgtgtcacagaatatcactccgtgagctttttggtttcttagaaagacagaaggtgggatttcaaaaggagagatgtttggcagtttggggataggattggggcaggggttgtggtcacacccagaaattgatgcctctccctcacacctctctgtgattttagactgggttatagctagccttcgcaggtctggtacttgctttgaagcccaggctagcttcaaacattcctcaatcttcctatcttagcctgtcaagtgttgggtggctcacatgtaccatcaagcccacacacagcgggaggtctcttcttgcacctcccttcccttctaggggtccagttggtctctcctgggtcccctggcagggcagccaaggtcaatgcaagggtgtgtctcctaagggagggcctgctctcttctggactggtcatgacctctggttactcatggggtggaaaagctactctcaggaatatgttttcctctcagaattttctttctaagttggcaagggctgagcccagtcagtgtcttgcagcaaaggccctggcccccaccttctggccttccctgttgaaggtttatggagcactagaggaatcggggacttcattttcttgaatagtcttaggtatggatgtacacattccgtgccctcttcccaccatgacaggatgtacagtggcttatagtgagagtgataatgacaactgtcttcaaatagtagagggtttggggtcctgttttgagaaggcttctgaggttgatccaatttgagcagaaaacatgcatattactttttggcttgtgccaatgagtacttgctcttgggagtgttcatgtattgtggagtgtgtgtgtatgtgtgtgtagtatgtatgtatgtatgtatgcatgcatgcatataagtatgtgtagtatgtgtgtgtaatatgtctgtatgtctgtgtagtgtgtatgcatatgtatatatgtgtgtatgtatagtatgtgtatgtagtatgtgtatatgttattatgagtgtgtatagtatgtatgtgtatagtgtatgtgcatgcatatataatgtatatgtatgtgtgcatatgtgtgtagtatgtgtacatgtgtgtgtgtagtaaatggcaaaatacagaaagacacagggcttggccttgtgcctaaggatcttgtgcctaaggtctgctccaggcaggtgacaagtcaaatgtaatggctattgggtaaggctgcagggagtaccacagtgttcctgagctcttatgccagcctgtggctcacacttgaatctggtgtgtgggagtaggtggaaaacaggcacgtgagggccactgaagctctttgctggtcaggagatgatggagaccataaaggtgacaaaggctttgcttccctcaggggacatgcgctccagcgagatgccggagctcacctccatgcacaccctctttgttcgagagcataaccggctggccacacagctcaagcgcctgaatcctcgatggaatggggagaagctctaccaggaggcccggaagattgtaggggccatggtccaggtaggctgtgtcctaacacatcttggccatgtgttcttgggcagtttttttttttaaatctctcctgaacactggcttttttttatccataaaaggctcaaaggagcgaaatgaaaatgcccctcagacatgagatgcttggcgagtgcacttcccttcctcctgtctccccggccctcttccttcctgagggcctgaggagttgagagggtcttaagcagaagcaccagagtcaccgctggctcttttacccgctgtcatcatcttgagcattcccccacccccacccccacccccaccccacccctgccacccggggcagggctcctccatctggtttttgatgttttcatgacccctaatttcccaagaatgtaaacattcgtgcatatcacacacacacacacacacaacacacacacacacacacacacacacatacacacactccctagactttagggagggggtgtggttagtgctgagcatggggttttgattgacaggcacacccacaagagggattcactttggtctcagaggcaattctagccttaggtccagagtgacactgtctcacagcctttgggggacatcgcactggagcagacctctgccatctctctgtctataaacctgtactgagccctactgtgtgctagtctattgctggacactgtgaataatctcagtaatttcaacttccctgggtctcaaggggaaatgggaccactaataccttaaagacacgggggatattacaatgactaaaaagacattttgtttaatcctgagagagagggagagagaattattccttgtttacatttgtggttcagagagtttaataaatgacctgaggtgagttctatttatgtccttctgttaaaatctaagacctaagactatgggagactgatcctagccaggtaggggcaattcccccaagctggagaccacagggaggaacaaatagacatctaacacaaagtgactttgttttagatcatcacataccgggactacctgcccttggtgttggggccagcagccatgaagaagtacctaccccagtaccgatcttacaacgactcagtagaccctcgaatcgccaatgtcttcaccaacgctttccgttatggccacaccctcatccaacccttcatgttccgcctgaacaatcagtaccggcccacagggcccaacccccgagtcccactcagcaaggtcttttttgccagctggagagtcgtgttggaaggtgaatggaacctaagaaaagccagcagctaatgatgggatggtggctgcagagacctaggctagggagaggtcttttttcatcttggggaacccagtcttgctttccactttcattgacttagaaggctagcgcatcccaaccttgtgaaccctcccccaaccctccaagtgttgcaagagcttatagcttctctctgacccctacgattccacttggaccagccctgactccactgtgtgctctcatagtaagagaaccaacctttcctttcactctggtctcttgtcctcaggtggcattgaccccatcctccgaggcctcatggccactccagccaaactgaatcgccagaatcaaattgtggtggatgagatccgggagcgactatttgagcaagtcatgaggataggactggatttgcctgctcttaacatgcagcgcagccgggatcacggcctcccaggtgagaggccagccacagccccatcccacctcccaggcagctccccttcttgctcaggggtcctttccagttttatggatgcacctgaggcaaggcgctgccttttccagcccctggtggcctaagttcggcctctcttggcttcttcacaagctggacttttgcaacccgcgtagcagctgaacatcatcccacgatctggaagtctgcctccatcgttagcacgttctgactcattcatgaactcgcgggtgggagagtaccactgtcatttttactgacgaggaaactagggcttagagcagttcagagtctggctcggtgtggttgccacgtcagtgagagacgtggaggttggacctaaggggatcagcggaacccaaaagccccgtgcctgtgttgtggagtagcacaagtggccacgcctctgggaaacgttttcattctctgagtcagagaaagatacattaggctgctgctctaagggacaatgtgctgtttccgtgcttgagtctggcgggatttgtggtaatacagggctggccagggtaggagagagtgaggaaaagagtgagaggtctggaggagcccagctgcctactttggcctgagccaatcatttagggatccattctacttacggtcttgtgtactgagatgggcttttgaacatctgtgtattctttcctgcccttcccagtactcctagccggcaggagaggggtccatttacagctacctgttctctggcccaacgcaggatacaatgcctggagacgcttttgtgggcttccacagcccagcacagtgggtgagctcggcacggtgctgaagaacctggagttggcacggaagctgatggcacaatatggcacgcccaacaacattgacatctggatgggcggtgtgtccgagcccctggagcccaatggccgtgtaggccagctccttgcctgcctcattggcactcagtttaggaagctacgtgatggtgatcggtaaggaagctgtgggaaagcagggtgccacaactgtgaggccgggagggggcgcatgcgcaccgcagcagtcaggcggcgcttagagaacaacttatctgtgagttggttccatcatgtctggtctagggatggtagtcaagttctcagccttggtggtacgtgcctttgcccactgaaccatcttgctggtcccaaaaatgccactcttaataggttctctgggtttctcagtgctactgtggaggcgtcagttaccatggaaaagtgccaaaagtctcccgctgaccccttttcatgtcatttcatcacgatcttggaatctccgtgtctagttcttatctagcttgcattacacctttggcaatatcgtaaaaaggtgtcccagtcaagaagagaacatcacaaatgccacccaggctttcttttaccccgtgggagcgtgcatcagactgggacccaagcagctgtgcttctgtcttctgggctgagtgggcactgcccacgacctgagctctcctgtgctgtagataagaatattctgaagccccctcacactcaggggacgtctcaagccaggggatgtcctgggtagcctcactcgggacatcctcagacataatgtccacgatgctccctcccatgcaggttttggtgggagaacccaggcgtgttcagtaaacagcagagacaggccttggccagcatctccttgccccgtatcatctgtgacaacactggcatcaccaccgtgtcgaagaacaacatcttcatgtccaacacatacccccgagactttgtcagctgtaacacccttcctaaactgaacctgacttcctggaaggagacctagaggttgggtaagggcttggcacactgaagggtatacctggttggctagtgggaatcacagagaactctcttccctgggtgaatccatctttgtggatattgtgggtgacagtggggggtggggggaaatgactggacattcacttgtgagtgcacaagtatatttgcatatacacttgtgcacatctatatatatgcaacatacataaactagtatttcatgtgtgtctggtctgtgagcatgggcaatgtttgatgtgccttgtgtgcctgccaatcacatgagtctgggctcactgcttacgacaatattaggaacgtggggacagtgagcagacagcccgagttcaagtcatcaccctgtagcacagtactgtgtgtgactttggccaaattactctaaggataaattctcttccttataaatgaaatcagccacggcttgttagagggcctattgcctcctccaactctccactcatggagcagctcccatgtgctaggcactgtgctagccatgggaagcagaaacaataaagatgaataccaaagatgaatgccaaatcctcgatcaccttctggttggcagaaacaaggtggtggcctccggaggagaggatgcagctagcagctgggctgtgtggttgtgatacagttctgggccagctctgcttaactggctatttctccttttcccttactctcttctcttgctattcacaggagtctgccgggagaaggacggaggccattggccttcctgtgctatttgtacctgaggttcacactaataaaacatggtggatgggga

SEQ ID NO: 139 - protein, 718 aa, Mus musculus
  source 1..718: note = MPO PROTEIN SEQ. ACCESSION: NP_034954
MKLLLALAGLLAPLAMLQTSNGATPALLGEVENSVVLSCMEEAKQLVDRAYKERRESIKRSLQSGSASPTELLFYFKQPVAGTRTAVRAADYLHVALDLLKRKLQPLWPRPFNVTDVLTPAQLNLLSVSSGCAYQDVRVTCPPNDKYRTITGHCNNRRSPTLGASNRAFVRWLPAEYEDGVSMPFGWTPGVNRNGFKVPLARQVSNAIVRFPNDQLTKDQERALMFMQWGQFLDHDITLTPEPATRFSFFTGLNCETSCLQQPPCFPLKIPPNDPRIKNQKDCIPFFRSCPACTRNNITIRNQINALTSFVDASGVYGSEDPLARKLRNLTNQLGLLAINTRFQDNGRALMPFDSLHDDPCLLTNRSARIPCFLAGDMRSSEMPELTSMHTLFVREHNRLATQLKRLNPRWNGEKLYQEARKIVGAMVQIITYRDYLPLVLGPAAMKKYLPQYRSYNDSVDPRIANVFTNAFRYGHTLIQPFMFRLNNQYRPTGPNPRVPLSKVFFASWRVVLEGGIDPILRGLMATPAKLNRQNQIVVDEIRERLFEQVMRIGLDLPALNMQRSRDHGLPGYNAWRRFCGLPQPSTVGELGTVLKNLELARKLMAQYGTPNNIDIWMGGVSEPLEPNGRVGQLLACLIGTQFRKLRDGDRFWWENPGVFSKQQRQALASISLPRIICDNTGITTVSKNNIFMSNTYPRDFVSCNTLPKLNLTSWKET

SEQ ID NO: 140 - DNA, 99 bp, synthetic construct
  source 1..99: mol_type = other DNA; note = Donor name: 3716-PD1ex1.HDR.3
  misc_feature 2: note = Phosphothyoate-modified
  misc_feature 3: note = phosphorthyoate modified
  misc_feature 99: note = phosphorthyoate modified
  misc_feature 98: note = phosphorthyoate modified
  misc_feature 1: note = 5' Biotin
cnnatcccacaggcgccctggccagtcgtctgggcgtttaattgagttgtcatatgttaataacggtgctacaactgggctggcggccaggatggttnn

SEQ ID NO: 141 - protein, 1691 aa, synthetic construct
  source 1..1691: note = BRCA2 peptide(n-term), ancestral dCasFok
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 142 - protein, 1655 aa, synthetic construct
  source 1..1655: note = DSS1 peptide(n-term), ancestral dCasFok
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 143 - protein, 1647 aa, synthetic construct
  source 1..1647: note = BRCA2 peptide 2(n-term), ancestral dCasFok
MAVTPKKKRKVFHTASGKKVKIAKESLDKRKNLFDEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 144 - protein, 1662 aa, synthetic construct; identical to SEQ ID NO: 32
  source 1..1662: note = RAD52 peptide(n-term), ancestral dCasFok

SEQ ID NO: 145 - protein, 1736 aa, synthetic construct
  source 1..1736: note = Streptavidin(n-term), ancestral dCasFok
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 146 - protein, 1662 aa, synthetic construct
  source 1..1662: note = BRCA2 peptide (c-term), ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 147 - protein, 1698 aa, synthetic construct
  source 1..1698: note = DSS1 peptide (c-term), ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGSEKKQPVDLGLLEEDDEFEEFPAEDWAGLDEDEDAHVWEDNWDDDNVEDDFSNQLRAELEKHGYKMETSSGGSKRTADGSEFESPKKKRKV

SEQ ID NO: 148 - protein, 1654 aa, synthetic construct
  source 1..1654: note = BRCA2 peptide 2 (c-term), ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 149 - protein, 1770 aa, synthetic construct
  source 1..1770: note = RAD54 peptide (c-term), ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHSGGSKRTADGSEFESPKKKRKV

SEQ ID NO: 150 - protein, 1669 aa, synthetic construct
  source 1..1669: note = RAD52 peptide (c-term), ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPPAPPVTHSTPVTVSEPLLEKDFLAGVTQELIKTLEDNSESGGSKRTADGSEFESPKKKRKV

SEQ ID NO: 151 - protein, 1759 aa, synthetic construct
  source 1..1759: note = Mdm2 peptide (c-term), ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVSGGSKRTADGSEFESPKKKRKV

SEQ ID NO: 152 - protein, 1743 aa, synthetic construct
  source 1..1743: note = Streptavidin (c-term), ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGGSKRTADGSEFESPKKKRKV

SEQ ID NO: 153 - protein, 1661 aa, synthetic construct
  source 1..1661: note = BRCA2 peptide(n-term), ancestral dCasFok, his-tagged
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 154 - protein, 1697 aa, synthetic construct
  source 1..1697: note = DSS1 peptide(n-term), ancestral dCasFok, his-tagged
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 155 - protein, 1653 aa, synthetic construct
  source 1..1653: note = BRCA2 peptide 2(n-term), ancestral dCasFok, his-tagged
MAVTPKKKRKVFHTASGKKVKIAKESLDKRKNLFDEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 156 - protein, 1769 aa, synthetic construct
  source 1..1769: note = RAD54 peptide(n-term), ancestral dCasFok, his-tagged
MAVTPKKKRKVRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 157 - protein, 1668 aa, synthetic construct
  source 1..1668: note = RAD52 peptide(n-term), ancestral dCasFok, his-tagged
MAVTPKKKRKVPPAPPVTHSTPVTVSEPLLEKDFLAGVTQELIKTLEDNSEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 158 - protein, 1742 aa, synthetic construct
  source 1..1742: note = Streptavidin(n-term), ancestral dCasFok, his-tagged
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 159 - protein, 1692 aa, synthetic construct
  source 1..1692: note = BRCA2 peptide (n and c-term), ancestral dCasFok
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 160 - protein, 1728 aa, synthetic construct
  source 1..1728: note = BRCA2 peptide (n-term),DSS1 peptide (c-term), ancestral dCasFok
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGSEKKQPVDLGLLEEDDEFEEFPAEDWAGLDEDEDAHVWEDNWDDDNVEDDFSNQLRAELEKHGYKMETSSGGSKRTADGSEFESPKKKRKV

SEQ ID NO: 161 - protein, 1684 aa, synthetic construct
  source 1..1684: note = BRCA2 peptide (n-term),BRCA2 peptide 2 (c-term), ancestral dCasFok
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 162 - protein, 1699 aa, synthetic construct
  source 1..1699: note = BRCA2 peptide (n-term), RAD52 peptide (c-term), ancestral dCasFok
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPPAPPVTHSTPVTVSEPLLEKDFLAGVTQELIKTLEDNSESGGSKRTADGSEFESPKKKRKV

SEQ ID NO: 163 - protein, 1789 aa, synthetic construct
  source 1..1789: note = BRCA2 peptide (n-term), Mdm2 peptide (c-term), ancestral dCasFok
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVSGGSKRTADGSEFESPKKKRKV

SEQ ID NO: 164 - protein, 1773 aa, synthetic construct
  source 1..1773: note = BRCA2 peptide (n-term), Streptavidin (c-term), ancestral dCasFok
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGGSKRTADGSEFESPKKKRKV

SEQ ID NO: 165 - protein, 1728 aa, synthetic construct
  source 1..1728: note = DSS1 peptide(n-term),BRCA2 peptide (c-term), ancestral dCasFok
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 166 - protein, 1720 aa, synthetic construct
  source 1..1720: note = DSS1 peptide(n-term), BRCA2 peptide 2 (c-term), ancestral dCasFok
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 167 - protein, 1836 aa, synthetic construct
  source 1..1836: note = DSS1 peptide(n-term), RAD54 peptide (c-term), ancestral dCasFok
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHSGGSKRTADGSEFESPKKKRKV

SEQ ID NO: 168 - protein, 1809 aa, synthetic construct
  source 1..1809: note = DSS1 peptide(n-term), Streptavidin (c-term), ancestral dCasFok
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGGSKRTADGSEFESPKKKRKV

SEQ ID NO: 169 - protein, 1773 aa, synthetic construct
  source 1..1773: note = Streptavidin(n-term), BRCA2 peptide (c-term), ancestral dCasFok
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 170 - protein, 1809 aa, synthetic construct
  source 1..1809: note = Streptavidin(n-term), DSS1 peptide (c-term), ancestral dCasFok
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGSEKKQPVDLGLLEEDDEFEEFPAEDWAGLDEDEDAHVWEDNWDDDNVEDDFSNQLRAELEKHGYKMETSSGGSKRTADGSEFESPKKKRKV

SEQ ID NO: 171 - protein, 1765 aa, synthetic construct
  source 1..1765: note = Streptavidin(n-term), BRCA2 peptide 2 (c-term), ancestral dCasFok
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 172 - protein, 1780 aa, synthetic construct
  source 1..1780: note = Streptavidin(n-term), RAD52 peptide (c-term), ancestral dCasFok
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPPAPPVTHSTPVTVSEPLLEKDFLAGVTQELIKTLEDNSESGGSKRTADGSEFESPKKKRKV

SEQ ID NO: 173 - protein, 1631 aa, synthetic construct
  source 1..1631: note = ancestral dCasFok, his-tagged
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 174 - protein, 1625 aa, synthetic construct
  source 1..1625: note = ancestral dCasFok
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 175 - DNA, 20 bp, synthetic construct
  source 1..20: mol_type = other DNA; note = #14106 for spCas9
ggcgccctggccagtcgtct

SEQ ID NO: 176 - DNA, 20 bp, synthetic construct
  source 1..20: mol_type = other DNA; note = guide RNAs #14082 for proteins of the invention
ctgtgggatctgcatgcctg

SEQ ID NO: 177 - DNA, 20 bp, synthetic construct
  source 1..20: mol_type = other DNA; note = guide RNAs #14082 for proteins of the invention
gtctgggcggtgctacaact

SEQ ID NO: 178 - RNA, 20 nt, synthetic construct
  source 1..20: mol_type = other RNA; note = PD1 exon1 sgRNA #3478
gtctgggcggtgctacaact

SEQ ID NO: 179 - RNA, 20 nt, synthetic construct
  source 1..20: mol_type = other RNA; note = PD1 exon1 sgRNA #3479
ctgtgggatctgcatgcctg

SEQ ID NO: 180 - RNA, 20 nt, synthetic construct
  source 1..20: mol_type = other RNA; note = PD1 exon1 sgRNA for Cas9 #3480
ggcgccctggccagtcgtct

SEQ ID NO: 181 - DNA, 1053 bp, synthetic construct
  source 1..1053: mol_type = other DNA; note = Blunt (B) NHEJ donor
aaaggtctcactgggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttactggtgagaccttt

SEQ ID NO: 182 - DNA, 1905 bp, synthetic construct
  source 1..1905: mol_type = other DNA; note = W HDR donor with SCNA binding sites
gcttaactatgcggcatcagacacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgcccactagtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttacagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacatggtcatagctgtttcctg

SEQ ID NO: 183 - DNA, 1875 bp, synthetic construct
  source 1..1875: mol_type = other DNA; note = dS HDR Donor
gcttaactatgcggcatcagacacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagactagtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacatggtcatagctgtttcctg

SEQ ID NO: 184 - DNA, 20 bp, synthetic construct
  source 1..20: mol_type = other DNA; note = 4004 RHA flanking Right (PD1rev)2
agcctgacccgtcattctac

SEQ ID NO: 185 - DNA, 20 bp, synthetic construct
  source 1..20: mol_type = other DNA; note = 4008 LHA flanking eGFPrev 2
gctgaacttgtggccgttta

SEQ ID NO: 186 - DNA, 20 bp, synthetic construct
  source 1..20: mol_type = other DNA; note = PD1 LHA SCNA-BS
caggcatgcagatcccacag

SEQ ID NO: 187 - DNA, 20 bp, synthetic construct
  source 1..20: mol_type = other DNA; note = PD1 RHA SCNA-BS
agttgtagcaccgcccagac

SEQ ID NO: 188 - DNA, 16026 bp, Homo sapiens
  source 1..16026: mol_type = unassigned DNA; note = PD-1 gene NG_012110.1 HOMO SAPIENS PROGRAMMED CELL DEATH 1 (PDCD1), REFSEQGENE ON CHROMOSOME 2
cctcagaaattgctgtgaatcaggcgctgcctccccaccgcggtgcccttggcccggcggctgcagcgtggtttccacccggccagccgggcgcccggcgcctcccactgcagaagcagtttcgggatggtcctgctgtttccagtgcttccccggaccgtccctgcgcggatggaaacttcccactggcccgagcgagcacggggattcccctcccctcgccactttccatcgctgctggctcatgttccagcacacggggcccatctcaccactgccgtgcgccgggcgccatgcgtcatgacgtgcaggctccttggaaactgtagggtttctcttctcagtccaggagcatgggagggcccccagcgaggggaaccttctccaggcccctgagagcaaggccccagtctcggaaagcagtcctagatcccctggcctcctgggcgctctggcccacagagggcttccggggcctttagtcatagtcaccaagaactggagtccacccagactcacttaaactgaagaatggctgactaaattgtggtgtggtacgtacatccacaccacagcctggatggctcggcacagagaggaacgggctgctgaaacacacgcgctggaggcatctccagggaatcccggaaggatgcgtactgcaccttcccacggctgtcgctggaatgaaacagtgatgggggtggagggcaggctcgtggccaccagcagggaggtgggtgtattgccaagggcacccggaggaccccgtggtgatggggacgttctgcagcttcgctgtcaggggatgcaggagccttcgcttgtgcccacattgtgcagcctttgataggcacacattagccagaaacggggactcaggatgggatcgaggtgtcacatcaaagtcattaacctggttgtgacgttgtcctgtggttttccaaactgttatcattcagagagactgagcagagtgtatgaggaaacatctgtgtaatttcttacaactgcaagtaaatctacaattatctcaattgtaaatgatacaatactcaaccaaaacatacaaccatcagccaggtgtggtggctcacgcctgtaatcccaactctttgggagtccaaggtgggagaattgctggaggcccggagtttgagaccagcctgggtaacataaagagacctcctctgcccccaccccaaattctacaaaaaaaaaaaatgttagatatgagctggccacggggtgcacgcctgtggtcccagctactcaggaggctgaggtcggaggatcgcttgagcccaggaggtcgaggctgcagtgagccaagatcacaccactgcactccagcctgggtgcagagcaagaccctgtctctaaaagaaaataaacagacaaaaaccacatacaactttgcttgttgtaaattatcttttaactgaatgccctggattgaatctggctgctgccatcccagggccagtgatttggatggggtatgaccctctgtgaggaaggagcaggcggtgggggaagggcctgggtgtccaggttccctgggaaggaaggctgagaaaaggagatgggggaggggtgcgcagggccggccagccaagggccccttagccccatctaccctgctccccggactccgcctgcctttcctcctcgtgacagaagacagtggaagcctactgggtggaaggcacgggcttaggatgtgtgtgggaggaaagtgtgtgtgctggggagcatgtatgtttgggagttgtgtgtgttggaaatcgtgtgttggggattgtgtgtatattgcagattttgtatgtgtgttggggattgtggtgtgtgggtgttgtagattgcgtgttggggattgtgttggggattgtgtatgtgttgggggttgtgtgtgtgttggggattgtgtgtgggggagattgtgtgtgtgtgctggggattgggtgtgttggggattgtgtgtgtgttgaggattgtgtgtgggggagattctgtgtgtgtgctggggattgggtgtgttggggattgtgtgtgtattgggaattgtgtgtgtgttgaggattgtgtgtgttggggatttgtgtgtgtgttggggattctgtgcatgttgggagttgtgtgtgagttggggacaatgtgtacagaggattgtgtgttggaaattttgtgtgtgcgttgggaattttgtgtatgtgttgtggattgcgtgtacgttggggattgtgcatgttgggaattttgtgtgtgtgttgagaattgtgtgtgagggaattgtgtgtgtttgagattgtgtgtgtattgggaattgtgtgtgtgttgaggattgtgtgtgttctgaggattgtatgtgttgggaattttgtgtgtgtgttgaggattgtgtgtgttggggattctacgtatgttgaaagttgtgtgtgtgttgggattgtgtgtgtgttgtggattgtgtgtgttgggaattgtgtgtgtgtgttgaggattgtgtgcagggggattgtgtgtgttggagattgtatgtgttgggaattttgtgtgtgtgttggggactgtgtatgttttggggattgtgtgtgttgggaattttgtgtgtgtgttgaggattgtgtgtggggggattgtgtgtgttggagattgtgtgtgtgttggggactgtgtgtgtgttggggactgtgtgtgttggggtgtggtgtgttggaaatcgtgtgttggggacaccgtatgtgtttgggggagggtgtcaataagtggtctggagtgtgatattggggtgcaggctccatgagtccccaccccacacctgagccctgggaccgcctatctgcttcggggtggggtcccaggaccctgtaggttcagcctactagtccaggcccaatgcccaatgcctgcatccctgcaggccctgtgctctccaggctcagacccctcgcagccctgcagaccctccctgggtccatgtgtctctttgcaggtgctccagcgagtagcaatgtggagagaccatcaggcagccctggcctcagtggccgcagtcccctggctccacgctgggcccaccccaccaggtctcctctcccatggcccaggggccttcagtgggactgagaggaggagggaaggagagtgggtgacagggaagaactgcagggagagaggagaggggtgggagaaggagaaggaaggaaggggtaggatggaagctgggtttctccctgtgcccgccccctactccaggacatgtgtccaagccctggcaggtggaattttgggggcagggccttggtggtgaggagaccttccaggggtctgatagcatctcccatctcagagcccacctcctgggcccagcctcccctccagcccacacagtggcattcccagtcctcagaggacagcttcgtcccacaaagctcagagccttgaggaaggcccactgctgccctggaacagagacagcattcaacagaggttggaacaaggctctacagggctgggggcagagggaggttctgtccagaatctgccttcaggacaagtacagccagcaggggcagcttagccacttatccactgcctgggcgaggcacagggctatggaggcacctaccaaccaacagttctccagccccagagccccagcccctgaggcacaagggtgggtgtgccaggagacagttgctgcgggccaccttagctgtctggcagcacagtgggtgctgccaggctccctgggggccccccgccaagcccacctggccagctgggccccccccacctccccaccaagccacccacacagcctcacatctctgagacccgggagtggccctttgttcataaacgagagcttcctcgccgtggccgcgcctcgcagacatcatctttgatgctctttttccactgtttcggtgctttaatgttttcccttcagagccgggccgagtgtctctcggagccaggcagccgcgccagctgtcaggcggtttctagcctcgcttcggttattttaagctgatgagcctgacgcatctcatcactaatatcagcagtttcatttctcctgttttccattcgctgtaataaaatgctcagcacagaatacaaggagataagcaagccatttcacaaacgccgggccgccagccaggcccaggcactggaccccctgaaccaccccaccctggcacgagtgggctggagggcagggccccggggaagaaggtcaaggctggaaggggaggtcagcctcacagccagcccctgccaccgccccagcccccccgtcaggctgttgcaggcatcacacggtggaaagatctggaactgtggccatggtgtgaggccatccacaaggtggaagctttgagggggagccgattagccatggacagttgtcattcagtagggtcacctgtgccccagcgaagggggatgggccgggaaggcagaggccaggcacctgcccccagcaggggcagaggctgtgggcagccgggaggctcccagaggctccgacagaatgggagtggggttgagcccacccctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctctgccttgcccaggcatccttggtcctcactcgagttttcctaaggatgggatgagccccatgtgggactaaccttggctttacgacgtcaaagtttagatgagctggtgatatttttctcattatatccaaagtgtacctgttcgagtgaggacagttcttctgtctccaggatccctcctgggtggggattgtgcccgcctgggtctctgcccagattccagggctctccccgagccctgttcagaccatccgtgggggaggccttggcctcactctcccggatcgaggagagagggagcctcttcctgggctgcccgtgaccctgggccctctgtgtacactgtgaccacagcccgctcctggaccctctgtgcccggctggccctctgtgcccagccagcctgcacctggggatgccaaggcctggggagggtggtttcacccaggccaagcctaagacagtccctctgggccctgctgggtaccggggtgtgacaccactgggaggacaagatgaggggcacccctggggccgccctgacaccccctcgaggctcctgccccgggggtcctggtgccccttcactgtggcaggcgactgggggttccccacctcggcccctctcccggggcctgctccccggcacctgaggcagcatccttgtcagggccgtgccttcctgcctcagcgccacctcttaaggttggcccgtgggtcactcaggactcagaactggagattctgggcaaaaggcaaagagcaaagggccaaaaggcatcccagggagacgactgcgggggaaccagagggcagaggggcgctcgtcacaggggagggggagctgagcgaggcaggaggggagccgagcctctccccccgtgtcccggctcttcaggcacgccctcgggacgccaccctccccgacccaggcgggaaagataagagcaaggtgtccgcagcctgacactcgtgcctcaggtgcccgcgcttgtgccggacaagactctcacaggtggcatgcctcggtttccccactggtaacagcacagggcactcagcaaggcgcagtgggcatgactggggtcctgtgggtcctgacccagatgtggccaccccggccgcagtggtcttcattccaggatgcctcttttccctcctgatctattcactgcgttcgccattcggtcattcccggggccaccactcccacctcaggtgtgtgcttcccttgtgttttatgagatatccccaacccggctgcttattggccccgtccgagggcaggagcataaataagagcctctgctttggcgtgggaccactgtgagctccagtcagcgctgccactgctgagctctgggccttcgacaggacttggccccttactgacttctccgtgtgctttgggtcatgggtgaggacgcctcctggcaaggctgcgtcctgaggattaaatcgggtcatctgtgaaaactacccagcccagcccctgacacttttttgtttgtttcttttagtgacagggtcttgctctgtcacccaggctggagtgcagtggtgtgatctcggctcactcgacctccggggctcaagcaattctcccacctctgcctccagagtagctgggactataggcacgtgccaccctgccaggctaatttcttccattttttttagagacagggtctcgctatgttccccaggctgggctcaaatggtcctcccacctcagcctccccaagtactgggattacaggcataagccactgcatctggcctccatgacacatatttttaaagtctgatttttaaagtcaaacttttgaagtcagattttaaacggactattttgaaaaatatacaaaaacgtttaaaaacaatgaatatccctcacctagaatcaataactaagaatattgacacatttgctttggggactgggcggctggagctgccatgacaaagctccgccgaccgagtggcttttaaacagagcctgccctctcgccgactgagggctggacgtgcaggatggagctccgcagggtcggctcccctgtgctctgaggggctctgctcagcctctcccggctgtggcttaaaaacagagcctgtcctcccgccgtggggggctggacatgcaggaccgaggggccacagggtcggctccctgtgctccgagagggctctgctcagcttctcctggctggggggttttgtggccaccctctgtgttcctgggttcagaagcatcccccaggctctgccttcatctctgcacgggtgactctgtacaggaagccaggcctgctggtcaatggccacccagccctgtgccctcatcttacctagtcccagctgccgtcaccctattcctaataaggccgccttctgaggtcatggggttaggacttccacataggaatctgtggggacacggttcggcccacagcccttcccacctccacacacacacacgactgtgaggagttggaagacctcactcctcacccctgccaggtcctctagggacaagctcgctgtcctcatcccagcacagcccgtgggacggtttccttgtccctaatgggaccacggtcagagatgccgggtctggtctgggccagcaggttcctccgcccggggcaggcagccttcttctgtgcgcttctggaaagcaatgtcctgtaatgcggtctctctgcgggagcacccccaccgccacctcacaggcctgttccacagccccgggatgggctctgtctccctcctgaccctgcatagggcacagccctctctcatcaacccacgatcctacgtggatccgagagggagcacctggggaaacaatggaatcccatagaaacaccccaaatctaacttgatccaggaccagccagtggtcacttctgaatattcaccttcctagtagacactaccagccaagggaggccaggaagccttcctggaggaggtggcctgaggactggggtgaggcaggccctgcgtgggggtcgccacccagcacccccacactgggtgggagccagtctctgagactggctgggggaggtgggagagggggctgcttgaactgcagacaccgaggtctagcccccaccccacccagccagttggtggaggcaggggaggccgaggggcccagctggacctgctccccggggtggattccaaaataggggggttggggggggcggaacaggagcccagggtcctggcttgaggcccagtggctgagggctggtgcaagccagacaggaaaagggttgagcctgtcagcgccagcacagatcaagtcaggagcaggtccctccaccaatgtgtgcaaataaatagcagctaagtttccagttacaagaacaatgcacagatggtcccagggacattgcggtgtggacacacagcggccattgtcctgtcgccagcacctcgccctacagctggggggtcccttagcacttcctagccatgcagggtccctgctcacagtacccgtgatgacttctgttcctcacctgcctgtctgtcccgacagctgcatggcagccctggcctgggagatggagaccccgaggggctgcctgcggtggtggggcccctgggtccccactgcattcccagaaacccagagggcagggcatttcccctgctctgtgccgagtccacccagccccagcctaggcccagtaagggctgcagcccaccctgtcccaggctgcctcccaggagccctcttggccctgatgccagaagcccatcttcctccattcaggcaggtctctgagtgccctggcctggctgcctgctggccctgagagtcacactaccccacagccctccttggtcaaaatccactctggagtggctggaagattccccgggcccacgccgcacacgcctatgcagggagcttcccctggccggccggcagacaagggcggtctcagagagggggctcacctcagcagccccttgtgtagctggccctcgcccctgccacctctgggaacaccaccaggaagctgggggacaggcacgcaggtgaaggaggcgagcgcttgtcagccgggaggccatgggcacagagggaacagggacaccctgggtggcctcaaggtcacttcaaacccctcactcgtcccctgggagggtgcccagtgaggttggcactaggagttggtcctggtcacatgacagacccacccacctctggtgtccagccagcacgccgtgggccagcctggctgcagggacacgagggcagcagccccctcctcctctgagctggttgctccttgagtcatcaccaccgcctgccacggaggccgcctgtcccaggaagcagagggaccgcagctgtggcaaccagggcctggtctctgtgtcacctcgctggggggccgtgcccaggcctgagacggaactgagtgacagtgcactgggtctgacagtgtggggctggcgccatgtttggggaaccctgtggcatgggacctgtgggtgagccgggaaaatcaccccgttgcatggcatctcgggcctggatcttaagcgcctgtgttggtgcctccgcctggcggaagagccgcgacccccacgttgccatgcgggtatcccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcctgcctgcggtctccaggtgtgcccggcccatgcgtgtgcccacctgcgagggcgtggggtgggcttggtcatttcttatcttacattggagacaggagagcttgaaaagtcacattttggaatcctaaatctgcaagaatgccagggacatttcagagggggacattgagccagagaggaggggtggtgtccccagatcacacagagggcagtggtgggacagctcagggtaagcagctcatagtggggggcccaggttcggtgccggtactgcagccaggctgtggagccgcgggcctccttcctgcggtgggccgtggggctgactccctctccctttctcctcaaagaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggagttctgtcctgtctggtggctgtgggggcagtgggcaggagggagggaggcctccagggagggacaaggcggcagccaggctgaggctgtgtgtggagggtgctggtggctcccctggatcctggcctgagtcaggccttcccctcgctgtcccagaaacacacaggccctcctcgccctgtttgccccactcacatgtagagccctccggtgaaaaattcctccaacatcacggggctggggacaccatcttccccaagcccaggccttttaggggggtcccacggccccctggccccaggcttcctggccaggcatgggtgtccctgagtgactgtgcctgctccatagcccccgtgtccccatgcctaccagtccctccgggcaggggtggtacctggtgccagggcgtccacagcggggtgggagggctgccctgggctatgagggacacagcatctgcgggtccctccctaagtacacagggtgggggcagcgggggaggcggggtcacgtctgctggagccctatcaccctgctgcggtccttggcagctagcagcagggacggtgcagggaaaggagccctgccaggccctcgggcaccgcagccaaacacccactgacaccccggggctcattctctgcttgccatcctggagcccagtcctggaccccatgcctgtgtcagggacaggcacagagacctcgcagctctgggaactcccggtgagatccggcagatcaggcctggcagggaacgctgcccgggggctctgcacatgatgtgggtttccatccgcaaaacccgataggtcccatggaaaatgaccacaggcaatgagaaagccatagagtgctgcctgtgagatcagcgtccagaactcacagctctcaaacacccactcacagcgcagcgcgccagcgtccagaattcacggctctcattcatccattgacagcgcagcgcgccagcatccagaactcaaggctctcaaacacccattcacagcgcagcgcgccagcatccagaactcacggctctcattcatccactgacagcgcagcgcgccagcatccagaactcaaggctctcaaacacccattcacagcacagcgcgccagcatccagaactcacggctctcattcatccattgacagcgcagcgcaccagcatccagaactcaaggctctcatacacccattcacagcgcagcgcgccagcatccagaactcaaggctttcattcatccattgacagcgcagcgcgccagcatccagaactcacgtctctcattcatccactgacagcgcagcgcgccagcatccagaactcaaggctctcaaacacccattcacagcgcagcgcgccagcatccagaactcaaggctctcattcatccattgacagcgcagcgcgccagcgtccagaactcacgtctctcattcatccactgacagcgcggcgcgccagcatccagaactcaaggctctcaaacacccattcacagcgcagcgcgccagcatccagaactcaaggctttcattcatccattgacagcgcagcgcgccagcatccagaactcaaggctctcaaacacccattcacagcgcagcgcgccagcatccagaactcaaggctctcaaacacccattcacagcgcagcgcgccagcatccagaactcaaggctctcattcatccattgtcagcgcagcgcgccagcatccagaactcaaggctctcaaacacccattcacagcgcagcgcgccagcatccagaactcaaggctctcatacacccattcacagcgcagcgcgccagcatccagaactcacgtctctcattcatccactgacagcgcagcgcgccagcatccagaactcaaggctctcattcatccattgacagcgcagcgcgccagcatccagaactcaaggctctcaaacacccattcacagcgcagcgcgccagcatccagaactcacggctctcattca

(19) United States
(12) Patent Application Publication
(10) Pub. No.: US 2024/0374758 A1
(43) Pub. Date: Nov. 14, 2024

(54) HOMOLOGY DNA REPAIR ENHANCEMENT OF CAS DERIVATIVES COMPOSITIONS AND USES THEREOF IN GENETIC MODULATION

(71) Applicant: TARGETGENE BIOTECHNOLOGIES LTD, DN Hefer (IL)

(72) Inventors: Dan Michael WEINTHAL, Rehovot (IL); Yoel Moshe SHIBOLETH, DN Hefer (IL); Talya KUNIK, Ramat Gan (IL); Devin Lee TRUDEAU, Rehovot (IL)

(21) Appl. No.: 18/683,907

(22) PCT Filed: Aug. 15, 2022
(86) PCT No.: PCT/IL2022/050887

§ 371 Date: Feb. 15, 2024

Related U.S. Application Data
(60) Provisional application No. 63/233,464, filed on Aug. 16, 2021.

(51) Int. Cl. A61K 48/00; C12N 9/22; C12N 15/11; C12N 15/85; C12N 15/90

(52) U.S. Cl. CPC A61K 48/005; C12N 9/22; C12N 15/111; C12N 15/85; C12N 15/907; C12N 2310/20

(57) ABSTRACT

The present disclosure provides nucleic acid guided genome modifier chimeric or fusion proteins, complexes or conjugate thereof, having enhanced homology-directed repair (HDR). The nucleic acid guided genome modifier disclosed herein comprise the following two components (a) at least one defective CRISPR-Cas protein (CRISPR-dCas) devoid of a nucleolytic activity or any variant or mutant thereof; and (b) at least one nucleic acid modifier component; and at least one of: (c) at least one donor attachment domain (DAD) for binding at least one Donor nucleic acid molecule; and (d) at least one repair factor recruitment domain (RFRD). The present disclosure further provides nucleic acid sequences encoding the nucleic acid guided genome modifier disclosed herein, as well as systems thereof and therapeutic and non-therapeutic methods thereof.

### TECHNOLOGICAL FIELD

The invention relates to genetic editing systems and methods. More specifically, the invention provides highly effective and versatile CRISPR/Cas protein variants, compositions, methods and uses thereof in gene editing by homologous recombination.

### BACKGROUND ART

References considered to be relevant as background to the presently disclosed subject matter are listed below:
Aird et al, 2018, Commun Biol, 1:54
Ali et al, 2020, Commun Biol, 3:44
Cai et al., 2020, Nat Metab. 2:934-945
Carreira et al., 2009, Cell, 136:1032-1043.
Charpentier et al, 2018, Nature Communications, 9:1133
Elhanani et al, 2020, Cell Reports. 31:107591.
Guoling et al, 2017, Sci Rep, 7:8943
Jayavaradhan et al, 2019, Nature Communications, 28:2866
Hu et al, 2018, Cell Biosci, 8:12
Keegan et al, 1986, Science, 231:699-704
Lee et al 2020 Cell Metabolism 31:822-36
Lei et al, 2003, Nature, 426:198-203
Lomova et al, 2019, Stem Cells, 37:284-294
Luo et al, 2020, Clinical Cancer Research, doi: 10.1158/1078-0432.CCR-20-0777
Pinder et al, 2015, Nucleic Acid Research, 43:9379-92
Rees et al, 2019, Nature Communications, 17:2212
Reuven et al, 2019, Biomolecules, 9:584
Robert et al, 2015, Genome Med, 7:93
Roche et al, 2018, CRISPR J, 1:414-430
Savic et al, 2018, Elife, e33761
Stefanovie et al., 2020, Nucleic Acids Res., 48:694-708.
Stayrook et al, 2008, Nature, 452:1022-25
Tenspolde et al, 2019, Journal of Autoimmunity, 103:102289
Tran et al, 2019, Frontiers in Genetics, 10:365
Radichev et al, 2020, Cellular Immunology, 104224

Acknowledgement of the above references herein is not to be inferred as meaning that these are in any way relevant to the patentability of the presently disclosed subject matter.

### BACKGROUND OF THE INVENTION

CRISPR-Cas endonucleases are RNA/protein complexes that specifically recognize target DNA sequences and cleave them.

In order to create precise genome modifications, homology-directed repair (HDR) may be used, whereby a Donor nucleic acid molecule has homology to the genomic target (homology arms) and sequences that are incorporated into the genomic DNA by homologous recombination following target DNA cleavage by a nuclease. There is therefore need in the art for improved CRISPR-Cas endonucleases displaying enhanced specificity and effectivity in genome modifications. Addition of HDR-enhancement domains to genome editing nucleases may improve their ability to induce HDR. Attachment of Donor nucleic acid molecule to the RNA-guided nuclease by “donor attachment domains” could potentially allow higher local concentration at the cleavage site and lower overall concentrations of nucleic acids in the transfected cell. This could reduce the hazard of illegitimate donor nucleic acid integration in random or off-target dsDNA breaks. Moreover, when used with Ribonucleoproteins (RNP), donor nucleic acids can potentially be calibrated stoichiometrically to match protein molarity avoiding or reducing free unattached nucleic acids.

The inventors have previously shown that dimeric genome editing system highly favors micro-homology-directed genome editing repair. This is due-to the use of type-II restriction nuclease subunit that dimerizes on the DNA cleavage site and digests it to leave “sticky” ends. Such sticky ends favor HDR repair pathway that may be induced by increasing local donor concentration or harboring HDR related components.

Here it is proposed to use a dimeric genome editing system to make a sticky double strand break (DSB) and combine such system with mechanisms to both increase local donor concentration and harbor the cellular HDR related components, in order to increase HDR repair mechanism to ensure safe, known end product genome editing.

HDR has been enhanced in the context of Cas9 using donor attachment domains in the following works: via SNAP-tag covalent linkage of DNA to Cas9 (Savic et al, 2018, Elife, e33761), via biotin-streptavidin non-covalent interaction (Roche et al, 2018, CRISPR J, 1:414-430), via HUH endonuclease-DNA covalent linkage (Aird et al, 2018, Commun Biol, 1:54), and by VirD2-DNA covalent linkage (Ali et al, 2020, Commun Biol, 3:44). HDR can also be enhanced by use of protein domains that are able to recruit cellular genomic DNA repair factors (“repair factor recruitment domain (RFRD)”), including Rad51 and Rad52, which mediate homology-based repair. Rad51 may be recruited by peptides derived from BRCA2 (Carreira et al., 2009, Cell, 136:1032-1043) (SEQ ID NO: 56). Rad52 may be recruited by peptides derived from DSS1 (Stefanovie et al., 2020, Nucleic Acids Res., 48:694-708) (SEQ ID NO:57).

HDR has also been enhanced in the context of Cas9 using repair factor recruitment domains in the following studies: fusion to 53BP1 (Jayavaradhan et al, 2019, Nature Communications, 28:2866), Rad51 (Rees et al, 2019, Nature Communications, 17:2212), CtIP (Charpentier et al, 2018, Nature Communications, 9:1133), Rad52 (Tran et al, 2019, Frontiers in Genetics, 10:365), Mre11 (Tran et al, 2019, Frontiers in Genetics, 10:365), HSV-1 alkaline nuclease (Reuven et al, 2019, Biomolecules, 9:584).

It was also shown that HDR may also be enhanced by controlling cell cycle so that the cells are in G2/S phase where homologous recombination is enhanced (Lomova et al, 2019, Stem Cells, 37:284-294), or by restricting nuclease activity to the G2/S phase of the cell cycle (Janssen et al, 2019, Mol Ther Nucleic Acids, 16:141-154).

Alternatively, small molecules may be used to enhance HDR or reduce NHEJ (Guoling et al, 2017, Sci Rep, 7:8943). Chemical inhibition of factors that repress HDR may also be employed (Wienert et al, 2020, Nature Communications, 11:2109) as well as chemical inhibition of NHEJ such as by Ligase IV inhibition (Hu et al, 2018, Cell Biosci, 8:12), or DNA-PKcs inhibition (Robert et al, 2015, Genome Med, 7:93). Rad51 activity may be stimulated, thereby promoting HDR (Pinder et al, 2015, Nucleic Acid Research, 43:9379-92). These previous studies were performed in the context of a Cas9 genome editing system. The present dimeric dCas-type IIS endonuclease-based genome editing system uses a different nuclease (e.g., FokI) which provides synergistic effect with HDR repair machinery, thus allowing extremely high HDR editing.

### SUMMARY OF THE INVENTION

In a first aspect, the present invention relates to a nucleic acid guided genome modifier chimeric or fusion protein, complex or conjugate having enhanced homology-directed repair. More specifically, the nucleic acid guided genome modifier protein of the invention may comprise: (a) at least one defective CRISPR-Cas (CRISPR-dCas) protein devoid of a nucleolytic activity or any variant or mutant thereof; and (b) at least one nucleic acid modifier component. The nucleic acid guided genome modifier chimeric or fusion protein of the present disclosure further comprises at least one of: (c) at least one donor attachment domain (DAD) for binding at least one Donor nucleic acid molecule; and/or (d) at least one repair factor recruitment domain (RFRD).

In a further aspect, the invention relates to a nucleic acid molecule comprising a nucleic acid sequence encoding at least one nucleic acid guided genome modifier chimeric protein having enhanced homology-directed repair or any variant, mutant, fusion/chimeric protein, complex or conjugate thereof. More specifically, the nucleic acid guided genome modifier chimeric protein encoded by the nucleic acid sequence of the invention may comprise: (a) at least one CRISPR-dCas protein devoid of a nucleolytic activity or any variant or mutant thereof; and (b) at least one nucleic acid modifier component. The nucleic acid guided genome modifier chimeric or fusion protein encoded by the nucleic acid sequence of the present disclosure further comprises at least one of: (c) at least one donor attachment domain (DAD) for binding at least one Donor nucleic acid molecule; and/or (d) at least one repair factor recruitment domain (RFRD).

In another aspect, the invention provides a nucleic acid guided genome modifier system having enhanced homology-directed repair. More specifically, the nucleic acid guided genome modifier system of the invention may comprise: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. It should be noted that the nucleic acid guided genome modifier chimeric or fusion protein of the disclosed system may comprise (i) at least one CRISPR-dCas protein devoid of a nucleolytic activity or any variant or mutant thereof; and (ii) at least one nucleic acid modifier component. The nucleic acid guided genome modifier chimeric or fusion protein of the disclosed system further comprises at least one of (iii) at least one DAD for binding at least one Donor nucleic acid molecule; and (iv) at least one RFRD. Specially, the system may further comprise at least one of: (b) at least one donor nucleic acid molecule; and (c) at least one target recognition element, or any nucleic acid sequence encoding the target recognition element.

In another aspect, the invention provides at least one cell, and in some embodiments any host cell, or ay population of cells comprising the cell in accordance with the invention. More specifically, the host cell of the invention may comprise and/or may be modified by, at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. The nucleic acid guided genome modifier chimeric or fusion protein comprised within, or modifying the cell of the present disclosure, may comprise (i) at least one defective CRISPR-Cas protein devoid of a nucleolytic activity or any variant or mutant thereof; and (ii) at least one nucleic acid modifier component. The nucleic acid guided genome modifier chimeric or fusion protein of the disclosed cell further comprises at least one of: (iii) at least one DAD for binding at least one Donor nucleic acid molecule; and/or (iv) at least one RFRD. The host cell may further comprise and/or modified by: (b) at least one donor nucleic acid molecule; (c) at least one target recognition element or any nucleic acid sequence encoding said target recognition element; (d) at least one nucleic acid cassette or any vector or vehicle comprising at least one of; (i) the nucleic acid sequence of (a); (ii) the nucleic acid sequence of (b); (iii) the nucleic acid sequence of (c); and (iv) the nucleic acid sequence of (a) and at least one of (b) and (c); and (e) at least one system comprising (a) and at least one of (b) and (c).

In a further aspect, the invention provides a composition. Specifically, the composition of the invention may comprise at least one of: (a) a nucleic acid guided genome modifier chimeric or fusion protein, complex or conjugate having enhanced homology-directed repair, or at least one nucleic acid sequence encoding said nucleic acid guided genome modifier chimeric or fusion protein. The nucleic acid guided genome modifier chimeric or fusion protein of the disclosed composition may comprise: (i) at least one CRISPR-dCas protein devoid of a nucleolytic activity or any variant or mutant thereof; and (ii) at least one nucleic acid modifier component. The chimeric or fusion protein of the disclosed composition further comprises at least one of: (iii) at least one donor attachment domain (DAD) for binding a Donor nucleic acid molecule; and/or (iv) at least one repair factor recruitment domain (RFRD). The composition of the invention may comprise: (b) at least one donor nucleic acid molecule; (c) at least one target recognition element or any nucleic acid sequence encoding said target recognition element; (d) at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c); (e) at least one system comprising (a) and at least one of (b) and (c); and (f) at least one cell comprising and/or modified by at least one of: the nucleic acid cassette or any vector or vehicle of (d) and the at least one system of (e); or any matrix, nano- or micro-particle comprising at least one of (a), (b), (c), (d), (e) and (f). The disclosed composition optionally further comprises at least one of pharmaceutically acceptable carrier/s, diluent/s, excipient/s and additive/s.

In a further aspect, the invention relates to a method of modifying at least one target nucleic acid sequence of interest in at least one cell. Specifically, the method may comprise the steps of contacting the cell with at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. More specifically, the nucleic acid guided genome modifier chimeric or fusion protein of the disclosed method may comprise: (i) at least one CRISPR-dCas protein devoid of a nucleolytic activity or any variant or mutant thereof; and (ii) at least one nucleic acid modifier component. The chimeric or fusion protein of the disclosed methods further comprises at least one of: (iii) at least one donor attachment domain (DAD) for binding a Donor nucleic acid molecule; and/or (iv) at least one repair factor recruitment domain (RFRD). The methods disclosed herein may further use (b), at least one donor nucleic acid molecule. In some embodiments, the donor molecule comprises at least one replacement sequence for incorporation in, and/or for replacing, the target nucleic acid sequence of interest; (c) at least one target recognition element or any nucleic acid sequence encoding the target recognition element. In some embodiments, the target recognition element specifically recognizes and binds the target sequence. In yet some alternative embodiments (d), the methods of the present disclosure may use at least one nucleic acid cassette or any vector or vehicle comprising at least one of: (i) the nucleic acid sequence of (a); (ii) the nucleic acid sequence of (b); (iii) the nucleic acid sequence of (c); and (iv) the nucleic acid sequence of (a) and at least one of (b) and (c). In yet some further embodiments, the disclosed method may use (e), at least one system or composition comprising (a) and at least one of (b) and (c).

In a further aspect, the invention provides a method of curing or treating, preventing, inhibiting, reducing, eliminating, protecting or delaying the onset of a pathologic disorder or condition in a subject in need thereof. Specifically, the method of the invention may comprise the steps of administering to the subject an effective amount of at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. More specifically, the nucleic acid guided genome modifier chimeric or fusion protein of the disclosed method may comprise: (i) at least one CRISPR-dCas protein devoid of a nucleolytic activity or any variant or mutant thereof; and (ii) at least one nucleic acid modifier component. The chimeric or fusion protein of the disclosed methods further comprises at least one of: (iii) at least one donor attachment domain (DAD) for binding a Donor nucleic acid molecule; and/or (iv) at least one repair factor recruitment domain (RFRD). The methods disclosed herein may further use (b), at least one donor nucleic acid molecule. In some embodiments, the donor molecule comprises at least one replacement sequence for incorporation in, and/or for replacing, a target nucleic acid sequence of interest in the genome of the treated subject. In some embodiments, such target sequence is associated directly or indirectly with the treated disorder. The methods disclosed herein may further use (c), at least one target recognition element or any nucleic acid sequence encoding the target recognition element. In some embodiments, the target recognition element specifically recognizes and binds the target sequence in the genome of at least one cell of the treated subject. In some alternative and/or additional embodiment, the methods disclosed herein may use (d), at least one nucleic acid cassette or any vector or vehicle comprising at least one of; (i) the nucleic acid sequence of (a); (i) the nucleic acid sequence of (b); (iii) the nucleic acid sequence of (c); and (iv) the nucleic acid sequence of (a) and at least one of (b) and (c). In some alternative and/or additional embodiment, the methods disclosed herein may use (e), at least one system comprising (a) and at least one of (b) and (c). In some alternative and/or additional embodiment, the methods disclosed herein may use (f), at least one cell and/or a population of cells comprising and/or modified by, at least one of: (a), (b), (c), (d) and (e). In yet some further alternative and/or additional embodiment, the methods disclosed herein may use (g), at least one composition comprising at least one of (a), (b), (c), (d), (e) and (f).

In a further aspect, the invention provides an effective amount of at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. More specifically, nucleic acid guided genome modifier chimeric or fusion protein may comprise: (i) at least one CRISPR-dCas protein devoid of a nucleolytic activity or any variant or mutant thereof; and (ii) at least one nucleic acid modifier component. The chimeric or fusion protein of the disclosed methods further comprises at least one of: (iii) at least one donor attachment domain (DAD) for binding a Donor nucleic acid molecule; and/or (iv) at least one repair factor recruitment domain (RFRD). (b) at least one donor nucleic acid molecule; (c) at least one target recognition element or any nucleic acid sequence encoding said target recognition element; (d) at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c); (e) at least one system comprising (a) and at least one of (b) and (c); (f) at least one cell comprising and/or modified by at least one of: (a), (b), (c), (d) and (e); and (g) at least one composition comprising at least one of (a), (b), (c), (d), (e) and (f); for use in method of curing or treating, preventing, inhibiting, reducing, eliminating, protecting or delaying the onset of a pathologic disorder or condition in a subject in need thereof. In some embodiments, the donor molecule comprises at least one replacement sequence for incorporation in, and/or for replacing, a target nucleic acid sequence of interest in the genome of the treated subject. In some embodiments, such target sequence is associated directly or indirectly with the treated disorder. Still further, in some embodiments, the target recognition element specifically recognizes and binds the target sequence in the genome of at least one cell of the treated subject.

In yet a further aspect, the invention provides an effective amount of at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. More specifically, nucleic acid guided genome modifier chimeric or fusion protein may comprise: (i) at least one CRISPR-dCas protein devoid of a nucleolytic activity or any variant or mutant thereof; and (ii) at least one nucleic acid modifier component. The chimeric or fusion protein of the disclosed methods further comprises at least one of: (iii) at least one donor attachment domain (DAD) for binding a Donor nucleic acid molecule; and/or (iv) at least one repair factor recruitment domain (RFRD). (b) at least one donor nucleic acid molecule; (c) at least one target recognition element or any nucleic acid sequence encoding said target recognition element; (d) at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c); (e) at least one system comprising (a) and at least one of (b) and (c); (f) at least one cell comprising and/or modified by at least one of: (a), (b), (c), (d) and (e); and (g) at least one composition comprising at least one of (a), (b), (c), (d), (e) and (f); for use in method of modifying at least one target nucleic acid sequence of interest in at least one cell.

In some embodiments, the donor molecule comprises at least one replacement sequence for incorporation in, and/or for replacing, the target nucleic acid sequence of interest. In some further embodiments, the target recognition element specifically recognizes and binds the target sequence

These and other aspect of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: HDR-enhanced Cas nucleoprotein comprising DAD

A schematic illustration of a protein comprising a nuclease monomer (Nuc), and a donor attachment domain (DAD), a nuclease-deficient Cas nucleoprotein (dCas) bound to a single guide RNA (sgRNA), bound to a DNA target site that is complementary to the sgRNA. The DAD is bound to a Donor nucleic acid molecule (Donor). Two monomers of this protein are bound to DNA target sites separated by a double-stranded gap region (dsDNA), positioning the nuclease domains for dimerization and cleavage (upper scheme). Following cleavage of the dsDNA, the Donor is used for homology directed repair (HDR) (lower scheme).

FIG. 2: HDR-enhanced Cas nucleoprotein comprising DAD and RFRD

A schematic illustration of a protein comprising a nuclease monomer (Nuc), a donor attachment domain (DAD), a repair factor recruitment domain (RFRD) and a nuclease-deficient Cas nucleoprotein (dCas) bound to a single guide RNA (sgRNA), bound to a DNA target site that is complementary to the sgRNA. The DAD is bound to a Donor nucleic acid (Donor). Two monomers of this protein are bound to DNA target sites separated by a double-stranded gap region (dsDNA), positioning the nuclease domains for dimerization and cleavage (upper scheme). Following cleavage of the dsDNA, the Donor is used for homology directed repair (HDR) (lower scheme). The RFRD recruits HDR-related factors that participate in the HDR process.

FIG. 3A-3E: HDR-enhanced gene replacement comprising DAD and RFRD

FIG. 3A-I-3A-II. The figure shows schematic presentation of the PD1 gene, the cut site and five dsDNA donor cassettes comprising an insert of 1025 bp encoding GFP in frame with the PD1 start codon. FIG. 3A-I shows HDR dsDNA donor cassettes, and FIG. 3A-II shows NHEJ dsDNA donor cassettes.

FIG. 3B. illustrates the PCR results comparing NHEJ vs HDR insertion in a human cell line

FIG. 3C. illustrates the PCR results comparing HDR donors with (W) or without (dS) a specific binding site (BS) for SCNAs in a human cell line.

FIG. 3D. illustrates the PCR results comparing HDR with nucleases comprising DAD and RFRD domains in primary human T-cells.

FIG. 3E. illustrates semi-quantitative PCR results comparing HDR with nucleases comprising DAD and RFRD domains in Human Hek293 cells.

Abbreviations: B (Blunt donor); O (Overhang donor); W (wSCNA donor—With SCNA 3478 and 3479 binding sites (BS) [3480 Cas9 guide 9 bp overlap+PAM with RHA, cannot cut]; bW (W with 5′-biotin on both ends); dS (dSCNA donor—without SCNA binding site [3480 no BS, 3479 11 bp overlap+PAM, 3478 10 bp+PAM); nD (no Donor DNA MCS—Multiple Cloning Site); pA (SV40 terminator & polyadenylation signal); LHA, RHA (Left and Right Homology Arms, respectively); Ex1 (exon 1); NN (No Nuclease GFP mRNA control); NE (Not Electroporated); NTC (No Template Control).

FIG. 4: HDR-enhanced Cas nucleoprotein comprising RFRD

A schematic illustration of a protein comprising a nuclease monomer (Nuc), a repair factor recruitment domain (RFRD), and a nuclease-deficient Cas nucleoprotein (dCas) bound to a single guide RNA (sgRNA), bound to a DNA target site that is complementary to the sgRNA. Two monomers of this protein are bound to DNA target sites separated by a double-stranded gap region (dsDNA), positioning the nuclease domains for dimerization and cleavage. The RFRD recruits HDR-related factors that participate in the HDR process which may involve a Donor nucleic acid (Donor), which may be supplied exogenously or from within the genome.

FIG. 5A-5C: Pure directional insertion—HDR insertion with no NHEJ

FIG. 5A. Experimental setup: the figure shows schematic presentation of exon1 of the PD1 gene, the cut site and the donor cassette used, that comprises an insert of 29nt and left and right homology arms (LHA, RHA, respectively).

FIG. 5B. PCR analysis: the figure illustrates the PCR reaction conducted.

FIG. 5C. presents the PCR results, showing HDR insertion without NHEJ.

Abbreviations: LHA—Left Homology Arm, RHA—Right Homology Arm, S—Sense strand, S—Sense strand. The samples: D9—TG #15151+guide pair; E9—TG #15172+guide pair; F9—TG #15190+guide pair; H9—spCas9+guide; A10—TG #15155 NO GUIDE control; Neg—no DNA.

### DETAILED DESCRIPTION OF THE INVENTION

The present invention provides a nucleic acid guided genome effector or modifier protein exhibiting enhanced homology-directed repair (HDR), as well as variants, mutants, fusion proteins and conjugates thereof. It should be noted that the effector or modifier protein of the invention may comprise at least one defective clustered regularly interspaced short palindromic repeats (CRISPR)-Cas (CRISPR-dCas) protein or Cas protein derived domain. In some embodiments, the PAM binding domain/PAM recognition motif of the Cas protein of the invention, may be deleted or replaced. The ability of the nucleic acid guided genome modifier chimera of invention to improve HDR induction provides systems, compositions and methods with enhanced specificity and effectivity in genome modifications.

Thus, in a first aspect, the present invention relates to a nucleic acid guided genome modifier chimeric or fusion protein, complex or conjugate having enhanced homology-directed repair. More specifically, the nucleic acid guided genome modifier protein of the invention may comprise: (a) at least one defective CRISPR-Cas (CRISPR-dCas) protein devoid of a nucleolytic activity or any variant or mutant thereof; and (b) at least one nucleic acid modifier component. The nucleic acid guided genome modifier chimeric or fusion protein of the present disclosure further comprises at least one of: (c) at least one donor attachment domain (DAD) for binding at least one Donor nucleic acid molecule; and/or (d) at least one repair factor recruitment domain (RFRD).

The present disclosure provides homology-directed repair (HDR) enhanced nucleic acid guided genome effector or modifier protein. Mammalian cells employ four distinct mechanisms to rescue themselves from double strand breaks (DSBs), specifically, NHEJ, HDR, alternative end-joining, and single-strand annealing. Among them, NHEJ and HDR are the two primary and competitive DNA repair pathways. NHEJ occurs throughout the cell cycle, whereas HDR operates predominately in the S and G2 phases. At the molecular level, the pathway of choice is based on DNA end resection, which is derived from the balance between end protection factors and end resection factors (e.g., 53BP1-RIF1 and BRCA1-CtIP). NHEJ is the preferred pathway, in most cases, to repair DSBs, and HDR only occurs in the supply of homologous DNA donors. The nucleic acid guided genome effector or modifier proteins of the present disclosure, enhance and increase the rate of an HDR repair. Specifically, display increased HDR, as compared to other nucleic acid guided genome effector or modifier proteins, or to other nucleic acid guided genome effector or modifier proteins that do not comprise the at least one donor attachment domain (DAD) for binding at least one Donor nucleic acid molecule; and/or the at least one repair factor recruitment domain (RFRD), and in some embodiments, both elements. As used herein “increasing”, “increased”, “increase”, “enhance” or “activate” are all used herein to generally mean an increase by a statistically significant amount; for the avoidance of any doubt, the terms “increased”, “increase”, “enhance” or “activate” means an increase of at least 10% in the level of HDR in the presence of the HDR enhanced nucleic acid guided genome effector or modifier proteins of the present disclosure, as compared to a reference level of the HDR reaction in the presence of other nucleic acid guided genome effector or modifier proteins, or to other nucleic acid guided genome effector or modifier proteins that do not comprise the at least one DAD, and/or the at least one RFRD. For example an increase of at least about 20%, or at least about 30%, or at least about 40%, or at least about 50%, or at least about 60%, or at least about 70%, or at least about 80%, or at least about 90% or up to and including a 100% increase or any increase between 1 0-100% as compared to a reference level, or at least about a 2-fold, or at least about a 3-fold, or at least about a 4-fold, or at least about a 5-fold or at least about a 10-fold increase, or any increase between 2-fold and 10-fold or greater in the level of HDR, in the presence of the HDR enhanced nucleic acid guided genome effector or modifier proteins of the present disclosure, as compared to a reference level of the HDR reaction in the presence of other nucleic acid guided genome effector or modifier proteins, or to other nucleic acid guided genome effector or modifier proteins that do not comprise the at least one DAD, and/or the at least one RFRD. As for the HDR reaction, as used by the present disclosure, the HDR reaction comprises the following steps, once the 3′ to 5′ exonuclease MRN complex (MRE11-RAD50-NBS1, named MRX for yeast) and C-terminal-binding protein interacting protein (CtIP) bind to the DSBs, the resection process is initiated, which leads to the generation of short 3′ single-stranded DNA (ssDNA) overhangs. In collaboration with Bloom syndrome protein helicase, the nucleases (including EXO1 and DNA2) execute extensive resection, which is essential for homology searching. The checkpoint kinases, ataxia telangiectasia mutated, RAD3-related protein, and cell cycle-dependent kinases are responsible for the post-translational modifications of the resection factors. The 3′ ssDNA overhangs are then bound by replication protein A, which protects the ssDNA from nucleolytic degradation and prevents the formation of secondary structures. With the help of mediator proteins, such as breast cancer type 2 susceptibility protein, the RecA-family recombinase, RAD51, forms a helical nucleoprotein filament on the ssDNA. This filament can interrogate intact duplex DNA for high sequence similarity (i.e., homology). Upon identifying homologous dsDNA, the RAD51 filament invades the duplex and pairs with the complementary strand, which is then utilized as a template for DNA synthesis to extend the 3′ end of the invading strand. The invading strand then dissociates, is extended, and is then able to anneal with the 3′ ssDNA overhang on the opposite side of the DSB. Following further DNA synthesis and ligation of the resultant nicks, HDR is complete.

In some embodiments, the DAD used in the nucleic acid guided genome modifier chimeric or fusion protein of the present disclosure, may be at least one of a sequence specific donor attachment domain, a non-sequence specific donor attachment domain and a covalent interaction domain. More specifically, as used herein a sequence specific interaction is meant that the DAD used in the nucleic acid guided genome modifier chimeric protein of the present disclosure, possess an affinity to bind to either double stranded or single stranded nucleic acid sequence in the donor molecule that may be in some embodiments sequence specific DNA binding, or alternatively, sequence non-specific DNA binding. In case of sequence specific DNA-protein interactions, a DAD as used herein binds to a nucleic acid sequence on a site having a specific nucleotide sequence. In case of sequence non-specific DNA protein interactions, the DNA binding protein can bind to a nucleic acid sequence in a random position on the nucleic acid sequence.

Still further, in some embodiments, the DAD used in the nucleic acid guided genome modifier chimeric or fusion protein of the present disclosure, may provide covalent interaction between the donor molecule and the nucleic acid guided genome modifier chimeric or fusion protein of the present disclosure. Covalent bonds involve the equal sharing of an electron pair by two atoms. Examples of important covalent bonds are peptide (amide) and disulfide bonds between amino acids, and C—C, C—O, and C—N bonds within amino acids. There are two types of covalent bond, non-polar that possess no charge, and polar, that possess a charge, and both are applicable in the present disclosure. In some further embodiments, the DAD may be a sequence specific DAD comprising at least one of a zinc finger DNA binding domain, a lambda repressor DNA binding domain, a Gal4 DNA binding domain and a protection of telomeres 1 protein (Pot1) ssDNA binding domain.

In some embodiments, the DAD of the nucleic acid guided genome modifier chimeric or fusion protein of the present disclosure, may comprise zinc finger DNA binding domain. More specifically, Zinc fingers are small protein domains that coordinate one or more zinc ions. Different ZFs can bind to and recognize DNA, RNA or proteins. DNA recognition can occur via sequence-specific and non-specific interactions, which are controlled by amino acids in the ZF-DNA interface (Bulyk, Huang, Choo, & Church, 2001, PNAS, 98:7158-63). Fusion of sequence-specific zinc fingers to functional domains like nucleases has been used to engineer sequence-specific nucleases (Tzfira et al., 2012, Plant Biotechnol J, 10:373-89), and sequence specific transcription-activators (Beerli, Dreier, & Barbas, 2000, PNAS, 97:1495-500). It should be noted however that in some embodiments of the invention, ZF used as DAD may provide a non-specific DNA binding activity. In some embodiments, such zinc finger applicable by the invention is a 24-residue zinc finger variant which has broad sequence specificity (non-stringent sequence requirement) and enhances non-specific binding to DNA (Chou et al, 2017, PLoS ONE, 12:e0175051). In yet some further specific embodiments, the Cys₂His₂ finger domains in testis zinc-finger protein may be applicable by the invention. Specifically, any one of Zif-QQR and Zif-QNK. In some particular and non-limiting embodiments, the zinc finger applicable by the invention may comprise the amino acid sequence as denoted by SEQ ID NO. 1, or any fragments, derivatives and variants thereof.

In some embodiments, a lambda repressor DNA binding domain may be used as a DAD in the nucleic acid guided genome effector or modifier proteins of the present disclosure. The λ repressor controls the expression of the viral genes by binding to six operator sites located within the left and right operator regions (O_{R} and O_{L}) of the λ chromosome. O_{R} and O_{L}, which are about 2500 bp apart, each contain three discrete 17 bp operator sites separated by 3-7 bp linkers. The repressor binds to each of these sites as a dimer. The λ repressor consists of two domains tethered by an about 40-residue linker. The N-terminal domain (NTD) comprises residues 1-92 and mediates binding of the repressor to the operator as well as its interaction with RNA polymerase. The C-terminal domain (CTD), residues 132-236, mediates dimerization as well as the interactions responsible for the cooperative binding of two repressor dimers to pairs of operator sites. In some specific embodiments lambda repressor DNA binding domain useful as a DAD in the present disclosure may comprise the amino acid sequence as denoted by SEQ ID NO: 2, and derivatives and variants thereof.

In some alternative or additional embodiments, DNA binding domain of the GAL4 transcription factor may be used as a DAD in the nucleic acid guided genome effector or modifier proteins of the present disclosure. GAL4 protein is a transcription factor from Saccharomyces cerevisiae required for the transcriptional activation of the genes encoding the galactose-metabolizing enzymes in response to galactose. The DNA binding domain of the GAL4 transcription factor from yeast is located in the N-terminal 60 residues of the polypeptide of 881 amino acids. This domain binds 2 Zn ions, which form a binuclear cluster, Zn2C6, with 6 C residues, two of which bridge the 2 metal ions. Still further, in some embodiments, a protection of telomeres 1 protein (Pot1) DNA binding domain may be used as a DAD in the nucleic acid guided genome effector or modifier proteins of the present disclosure. More specifically, the telomeric single-strand DNA binding protein protection of telomeres 1 (POT1) protects telomeres from rapid degradation in Schizosaccharomyces pombe and has been implicated in positive and negative telomere length regulation in humans. Human POT1 appears to interact with telomeres both through direct binding to the 3′ overhanging 0-strand DNA and through interaction with the TRF1 duplex telomere DNA binding complex. In some embodiments, the pot1 DNA binding domain useful as a DAD in the present disclosure may comprise the amino acid sequence as denoted by SEQ ID NO: 3, and derivatives and variants thereof. In some further embodiments, the DAD may be a covalent interaction domain comprising a virD2 domain. VirD2 is one of the key Agrobacterium tumefaciens proteins involved in T-DNA processing and transfer. In addition to its endonuclease domain, VirD2 contains a bipartite C-terminal nuclear localization sequence (NLS) and a conserved region called omega that is important for virulence.

In some embodiments, VirD2 useful as DAD that provides covalent interaction between the donor and the nucleic acid guided genome modifier chimeric or fusion protein of the present disclosure, may comprise the amino acid sequence as denoted by SEQ ID NO: 4, or any homologs or variants thereof.

In some further embodiments, the DAD may be a non-sequence specific donor attachment domain, for example, at least one domain of an affinity pair.

The association, binding, and/or attachment between the donor nucleic acid molecule, or modified moieties thereof that act as binding sites to the DAD, and the nucleic acid guided genome modifier chimeric protein of the present disclosure may involve a non-sequence specific interaction, as disclosed above.

The optional use of a non-sequence specific DAD may provide a flexible use of a single DAD as an adaptor for various donor nucleic acid molecules that comprise the corresponding binding member of the binding pair used. Thereby creating a “universal” nucleic acid guided genome modifier chimeric or fusion protein, which is suitable to various donor molecule/s that comprise the appropriate member of the binding pair used. More specifically, nucleic acid guided genome modifier chimeric proteins of the invention thereof comprising a non-specific DAD, may bind any donor nucleic acid molecule that comprise an attachment domain comprising the corresponding binding member of the binding pair used. For example, a nucleic acid guided genome modifier chimeric protein that comprise a Streptavidin domain binds any donor nucleic acid molecule that comprise a biotinylated attachment domain.

In some embodiments, such interactions may include the following pairs: Biotin-Avidin; Biotin-Streptavidin; Biotin-modified forms of Avidin; Protein-protein interactions; protein-nucleic acid interactions; ligand-receptor interactions; ligand-substrate interactions; antibody-antigen interactions; single chain antibody-antigen; antibody or single chain antibody-hapten interactions; hormone-hormone binding protein; receptor-agonist; receptor-receptor antagonist; anti-Fluorescein single-chain variable fragment antibody (anti-FAM ScFV)-Fluorescein; anti-DIG single-chain variable fragment (scFv) immunoglobin (DIG-ScFv)-Digoxigenin (DIG); IgG-protein A; enzyme-enzyme cofactor; enzyme-enzyme inhibitor; single-strand DNA-VirE2; StickyC-dsDNA; RISC-RNA; viral coat protein-nucleic acid. In some embodiments, the affinity pair is avidin-biotin (e.g., streptavidin domain for biotinylated donor molecules). In yet some further embodiments, any tag-anti-tag pair (including antigen-antibody, or ligand-receptor may be used). In some particular embodiments, non-sequence specific donor attachment domain may comprise a streptavidin domain. In yet some further embodiments, binding-pair may further include Agrobacterium VirD2-VirD2 binding protein; antibody-antigen; single chain antibody-antigen interaction; anti-Fluorescein single-chain variable fragment antibody (anti-FAM ScFV)-Fluorescein; anti-DIG single-chain variable fragment (scFv) immunoglobin (DIG-ScFv)-Digoxigenin (DIG) and IgG-protein A.

Still further, in some embodiments, the at least one Donor nucleic acid molecule may be attached to the nucleic acid guided genome modifier chimeric protein of the present disclosure via any DAD discussed herein. In some embodiments the recognition region of the Donor nucleic acid molecule may comprise a chemical modification selected from the group consisting of 5′-end modification, 3′-end modification, and internal modification. In yet some further embodiments, such chemical modification may be any one of a nucleotide modification, and addition of a non-nucleotide moiety. Still further, non-nucleotide moiety may be any one of Biotin, Fluorescein, Amine-linkers, oligopeptides, Aminoallyl, a dye molecule, fluorophores, Digoxigenin, Acrydite, Adenylation, Azide, NHS-Ester, Cholesteryl-TEG, Alkynes, Photocleavable Biotin, Thiol, Dithiol.

In yet some further embodiments, the nucleotide modification may be any one of phosphate, 2-Aminopurine, Trimer-20, 2,6-Diaminopurine, 5-Bromo-deoxiUridine, DeoxiUridine, Inverted dT, dideoxi-nucleotides, 5-methyl deoxyCytidine, deoxyInosine, 5-nitroindole, 2-O-methyl RNA bases, Iso-dC, Iso-dG, Fluorine modified bases and Phosphorothioate bonds.

In yet another embodiment, the nucleic acid guided genome modifier chimeric or fusion protein of the present disclosure comprises at least one repair factor recruitment domain (RFRD). More specifically, DNA damage leads to gene mutations and genomic instability. One of the earliest events in the DNA damage response (DDR) is the recruitment of poly (ADP-ribose) polymerase 1 (PARP1) to the site of damage through its binding to DNA, resulting in its activation and the synthesis of poly(ADP-ribose) (PAR) chains on histones, non-histone proteins, and on itself. PARylation is involved in the early recruitment of DDR proteins, such as the sensor complex MRN (Mre11, Rad50, Nbs1) that in turn recruits ATM to breaks leading to its activation. ATM initiates a series of phosphorylation events including the phosphorylation of histone variant H2AX on serine 139 (also termed gH2AX). Phosphorylation of H2AX by the PI3-K-like kinases ATM, ATR, and DNA-PKcs is required for DNA damage signal amplification and subsequent accumulation of numerous DDR proteins at double-strand break (DSB) sites, including 53BP1, MDC1, and specific components of the main repair pathways homologous recombination (HR) and non-homologous end joining (NHEJ). In addition, DNA damage checkpoint factors (DDCFs) play a crucial role by arresting the cell cycle to allow the time to repair once a damage to DNA has taken place. The detailed characterization of DDCFs has classified as many as 26 human proteins into four categories including DNA damage sensors, mediators, transducers and effectors. Additionally, DNA damage repair machinery is composed of 42 DNA damage repair factors (DDRFs). DDRFs are grouped into eight subgroups based on their DNA repairing mechanisms such as base excision repair, nucleotide excision repair, homologous recombination repair, non-homologous end joining, microhomology-mediated end-joining, mismatch repair, and shared mechanism subgroups.

Thus, in some embodiments, RFRD of the nucleic acid guided genome modifier protein of the invention may recruit any DDR protein, specifically, any protein involved in the HDR pathway of double strand breaks (DSBs).

In some specific embodiments, the protein involved in HDR may be at least one of a Recombination Protein A (Rad) family member, a Fanconi Anemia Core Complex member, Tumor Suppressor p53, or C-Terminal-Binding Protein-Interacting Protein (CtIP).

Additional suitable repair factors may be any one of Breast Cancer 1 (BRCA1), Breast Cancer 2 (BRCA2), Rad50, Rad51, Rad52, Rad54, Ataxia telangiectasia mutated (ATM), H2A histone family member X (H2AX), DNA damage checkpoint protein 1 (MDC1), Mre11, Nbs1, C-terminal-binding protein-interacting protein (CtIP), exonuclease 1-Bloom helicase (Exo1-BLM), replication protein A (RPA), proliferating cell nuclear antigen (PCNA), 53BP1, HSV-1 alkaline nuclease.

In some specific embodiments, the repair factor recruitment domain may comprise the BRCA2 protein, or any fragment or peptides thereof, for recruitment of Rad51 and Rad52. In some embodiments, the RFRD may comprise the DSS1 protein, or any fragment or peptides thereof for recruitment of Rad52. Still further, in some embodiments, RFRD may comprise the RAD52 protein, or any fragment or peptides thereof, or the RAD54 protein, or any fragment or peptides thereof.

In some particular embodiments, the fragment of BRCA2 may comprise residues 1543-1575 of the BRCA2 amino acid sequence as denoted by SEQ ID NO: 56, or any variants or homologs thereof.

In some embodiments, the fragment of DSS1 comprise the residues 2-70 of the DSS1 amino acid sequence, as denoted by SEQ ID NO: 57, or any variants or homologs thereof. In some embodiments, the fragment of RAD54 may comprise in some embodiments, residues 2-142 of the RAD54 N-terminal peptide that comprises the amino acid sequence as denoted by SEQ ID NO: 29, or any variants or homologs thereof. In some specific embodiments, the nucleic acid guided genome modifier chimeric or fusion protein of the present disclosure may comprise as an RFRD of the RAD54 as disclosed herein. Particular embodiments for such nucleic acid guided genome modifier chimeric or fusion protein may include the RAD54 peptide (n-term), ancestral dCasFok, his-tagged chimera that comprises the amino acid sequence of SEQ ID NO: 156, that comprises at the N-terminal end thereof, the RAD54 fragment residues 12-152 of or SEQ ID NO: 29 (N′). In yet some other embodiments, the nucleic acid guided genome modifier chimeric or fusion protein of the present disclosure may comprise an RFRD of the RAD54 in the C-terminal end thereof, for example, the RAD54 peptide (c-term), ancestral dCasFok chimera that comprises the amino acid sequence of SEQ ID NO: 149, with residues 1608-1748 derived from RAD54 (C′). Still further additional embodiments for the nucleic acid guided genome modifier chimeric or fusion protein of the present disclosure, may be the DSS1 peptide (n-term), RAD54 peptide (c-term), ancestral dCasFok chimera, which comprises the amino acid sequence of SEQ ID NO: 167, where the RAD54 derived sequence is residues 1674-1814 (C′).

Still further, as discussed above, one of the major components of the nucleic acid guided genome modifier chimeric or fusion protein of the present disclosure is a CRISPR-Cas protein, specifically, a defective Cas protein.

The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the foreign DNA, called proto-spacers, which are subsequently cleaved. The spacers, or other suitable constructs or RNAs can be rationally designed and produced to target any DNA sequence. Moreover, this recognition element may be designed separately to recognize and target any desired target including outside of a bacterium.

“Complement” or “complementary” as used herein means Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules. Partial complementary may mean less than 100% complementarity, for example 80% complementarity,

In some specific embodiment, the nucleic acid guided genome modifier chimeric or fusion protein, complex or conjugate having enhanced homology-directed repair (HDR) in accordance with the present disclosure, or any variants thereof (for example, PAM reduced or abolished variants thereof), may be of a CRISPR Class 2 system. In yet some further particular embodiments, such class 2 system may be any one of CRISPR type II, and type V systems. In certain embodiments, the Cas applicable in the present invention may be any Cas protein of the CRISPR type II system. In more specific embodiments, the nucleic acid guided DNA binding protein nuclease may be CRISPR-associated endonuclease 9 (Cas9) system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein. It should be appreciated that any type II CRISPR-Cas systems may be applicable in the present invention, specifically, any one of type II-A or B. Thus, in yet some further and alternative embodiments, at least one cas gene used in the methods and systems of the invention may be at least one cas gene of type II CRISPR system (either typeII-A or typeII-B). In more particular embodiments, at least one cas gene of type II CRISPR system used by the methods and systems of the invention may be the cas9 gene.

Thus, according to such embodiments, the nucleic acid guided genome modifier chimeric or fusion protein of the present disclosure is a CRISPR-associated endonuclease 9 (Cas9). Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand breaks (DSBs).

CRISPR type II system as used herein requires the inclusion of two essential components: a “guide” RNA (gRNA) and a CRISPR-associated endonuclease (Cas9). The guide molecules suitable in the present disclosure will be defined herein after.

In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified. It should be noted that any CRISPR/Cas proteins may be used by the invention. In some embodiments of the present disclosure, the endonuclease may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CHI, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

In some embodiments, Cas protein suitable for the nucleic acid guided genome modifier chimeric protein, complex or conjugate of the present disclosure may be at least one of Cas9, CasX, Cas12a1, CasF, Cas14a1, an ancestral Cas and Cas14b5.

In some other embodiments, at least one of the PAM Binding Domain (PBD) of the CRISPR-dCas protein used in the nucleic acid guided genome modifier chimeric or fusion protein of the present disclosure, has reduced or abolished Protospacer Adjacent Motif (PAM) constraint. In some embodiments, at least one of the PAM binding domain (PBD) and/or PAM recognition motif, any fragment of said PBD, and at least one amino acid residue adjacent to said PBD, of the CRISPR-dCas protein is deleted or replaced. As used herein, a “PAM binding domain” or a “PAM binding motif” of the CRISPR-Cas protein used in the HDR enhanced nucleic acid guided genome modifier chimeric or fusion protein of the present disclosure, refers to any amino acid residue or to any sequence, secondary structure and/or three dimensional tertiary structure (formed by either proximate or distant residues) that is involved or participates directly or indirectly in recognition and binding of the PAM in the target nucleic acid sequence. The “PAM binding domain” or a “PAM binding motif” deleted in such Cas variant may therefore comprise at least one amino acid residue, a linear peptide composed of two or more residues, any secondary or three dimensional tertiary structure formed by at least two amino acid residues located either in close proximity in a linear sequence or located at distant parts or domain of the protein. In some embodiments, the “PAM binding domain” or a “PAM binding motif” deleted or replaced in the CRISPR-Cas protein comprised within the HDR enhanced nucleic acid guided genome modifier chimeric or fusion protein of the present disclosure may involve residues derived from the N-terminal and/or the C-terminal parts of the proteins forming a structure that participates in PAM binding and recognition. The “PAM binding domain” or the “PAM binding motif” deleted or replaced in the CRISPR-Cas protein of the HDR enhanced nucleic acid guided genome modifier chimeric or fusion protein of the present disclosure, may comprise in some embodiments at least one of loop/s, alpha helix/helices, beta sheet/s and any combinations thereof. In some embodiments, the “PAM binding domain” or “PAM binding motif” deleted or replaced in the Cas protein used by the HDR enhanced nucleic acid guided genome modifier chimeric or fusion protein of the present disclosure may comprise at least two loop structures. More specifically, such loops may include in some embodiments a loop referred to herein as the “PAM BD loop” that is comprised within the PAM binding domain of the Cas protein (derived from the C′ terminal part of the protein), and at least one additional structure derived from a distant part of the Cas protein (the N′ terminal part of the protein), for example, a loop structure referred to herein as the “ScLoop”. In some embodiments, the “PAM binding domain” in accordance with the present disclosure comprises the PAM BD loop. Such domain may comprise residues from about position 1108+/−10 amino acid residues, to about position 1375+/−10 amino acid residues.

In some embodiments, the Cas protein used in the HDR enhanced nucleic acid guided genome modifier chimeric or fusion protein of the present disclosure may be at least one of ScCas9, SpCas9, CasF-1, CasF-2, CasF-3, and deltaproteobacteria CasX, and wherein at least one PAM interacting Arginine and/or lysine residue of the PBD of the indicated Cas protein is deleted or replaced.

In some specific embodiment, the Cas protein suitable for the HDR enhanced nucleic acid guided genome modifier chimeric or fusion protein of the present disclosure may be ScCas9.

In some embodiments, ScCas9 may comprise an amino acid sequence as denoted by SEQ ID NO. 123, with a replacement or deletion of at least one of: residues Thr1330 to Arg1342, residues Ile367 to Ala376 and residues Lys1337 and Gln1338.

In some further embodiments in such PAM reduced or PAM abolished Cas protein of the HDR enhanced nucleic acid guided genome modifier chimeric or fusion protein of the present disclosure, the PAM binding domain and/or at least one adjacent amino acid residues may be replaced by at least one Non-Specific DNA Binding Domain (NSBD).

In some embodiments, the NSBD may be at least one Double-Stranded DNA binding domain or protein (dsDBP), and any variant and fragments thereof, as discussed herein before.

In some embodiments, the least one dsDBP may be at least one of: at least one Zinc finger (ZF), Helix-turn-helix (HTH), SRC Homology 3 (SH3) domain, chromatin-binding domain (CBD) protein and Sticky-C(StkC), domain or protein, and any variant and fragments thereof.

In some more specific embodiments, the ZF domain or protein may be at least one Cys₂His₂ TZD, the HTH domain or protein may comprise Lac repressor residues 1 to 46, the SH3 domain may comprise at least one of: residues 219 to 270 of HIV integrase protein residues 1 to 64 of the Sso7D DNA-binding protein of Sulfolobus solfataricus, and residues 1 to 64 of the Sto7D DNA-binding protein from Sulfolobus tokodaii, the StkC domain may comprise residues 232-305 of Arabidopsis MBD7 methyl-CpG-binding domain, the CBD may comprise at least one High Mobility Group (HMG) protein, and the HMG protein may be any one of HMGA, HMGB and HMGN.

In yet some further embodiments, the Helix-turn-helix (HTH) domain may be used as DBP (DNA binding domain or protein) by the invention. More specifically, the helix-turn-helix domain, as used herein, is comprised of two helices that bind to and recognize DNA, separated by a short turn motif. They may be found in proteins involved in DNA transcription regulation and other activities. The helix-turn-helix (HTH) domain can include two or more helices, as well as beta sheet domains. For example, the “winged helix-turn-helix” domain comprises a 3-helical bundle followed by a 3-stranded beta sheet.

In yet some further specific embodiments, the HTH applicable in the present disclosure may comprise Lac repressor (LacI) residues 1 to 46, known to fold independently, bind non-specifically, and facilitate diffusion along DNA (Kalodimos et al, 2004, Science 305:386-9). In some particular and non-limiting embodiments, Lac repressor residues 1 to 46, may comprise the amino acid sequence as denoted by SEQ ID NO. 124, or any fragments, derivatives and variants thereof.

Still further, in some further embodiments, at least one SH3 domain may be used as DBP by the invention. More specifically, the five-stranded beta-barrel is a protein motif composed of five beta-strands (also known as a “SRC homology 3 domain” or SH3 domain). In the HIV integrase, a five-stranded beta-barrel mediates non-specific binding to DNA. One beta-barrel motif described here comprises residues 219 to 270 of the HIV integrase protein. In some particular and non-limiting embodiments, HIV integrase residues 219 to 270, may comprise the amino acid sequence as denoted by SEQ ID NO. 125, or any fragments, derivatives and variants thereof.

In yet some further embodiments, a SRC Homology 3 (SH3) SH3 domain-like protein applicable in the present invention as DBP may comprise the Sso7D from Sulfolobus solfataricus. More specifically, residues 1 to 64, of the Sso7D, which also has been found to mediate non-specific DNA binding interactions (Kalichuk et al, 2016, Scientific Reports 6:37274), may be used as non-specific DBP in accordance with the invention. In some particular embodiments, residues 1 to 64 of Sso7D may comprise the amino acid sequence as denoted by SEQ ID NO. 126, or any fragments, derivatives and variants thereof.

In certain embodiments, the Sto7D from Sulfolobus tokodaii, may be used as DBP by the present invention. In more specific embodiments, residues 1 to 64 of the Sto7D, may be used, more specifically, residues 1 to 64 that comprise the amino acid sequence as denoted by SEQ ID NO. 127, or any fragments, derivatives and variants thereof.

Still further, in some embodiments, CBDs (chromatin-binding domains), may be used by the invention as DBP s. More specifically, Chromatin is a structure formed by the assembly of DNA and proteins. Chromatin-binding proteins interact with DNA in the context of chromatin and may be involved in forming and regulating the condensed structure, which can govern DNA accessibility to transcription, replication, and other functions. Non-limiting examples for CBDs applicable in the present disclosure include HMGs and StkCs.

In some further specific embodiments, HMGs (high mobility group proteins) may be used as DBP by the present invention. High mobility group proteins are chromosomal proteins involved in DNA replication, recombination, repair, and transcription. These proteins can bind to and alter chromatin structure, and comprise three families: HMGA, HMGB, and HMGN (Reeves, 2010, Biochim Biophys Acta, 1799(1-2):3). For example, the HMGB family are alpha helical protein domains, which can bind to the minor groove of DNA in a non-sequence specific manner and can bend DNA. Transient interactions of HMGB with DNA may mediate stable interactions of transcription factors with their DNA targets (Agresti & Bianchi, 2003, Curr Opin Genet Dev, 13:170-8).

In some specific embodiments, the HMGB protein used herein as DBP may comprise residues 2 to 79 of human HMGB4. In further particular embodiments, such domain may comprise the amino acid sequence as denoted by SEQ ID NO. 128, or any fragments, derivatives and variants thereof.

In some specific embodiments, the HMGN protein used herein as DBP may comprise residues 1 to 100 of human HMGN. In further particular embodiments, such domain may comprise the amino acid sequence as denoted by SEQ ID NO. 129, or any fragments, derivatives and variants thereof.

In some specific embodiments, the HMGN protein used herein may comprise residues 1 to 100 of human HMGB1. In further particular embodiments, such domain may comprise the amino acid sequence as denoted by SEQ ID NO. 130, or any fragments, derivatives and variants thereof.

In some specific embodiments, the HMGN protein used herein as DBP may comprise residues 1 to 100 of human HMGB3. In further particular embodiments, such domain may comprise the amino acid sequence as denoted by SEQ ID NO. 131, or any fragments, derivatives and variants thereof.

Still further, in some embodiments, Sticky-C(StkC) may be used as DBPs by the present invention. More specifically, the C-terminal chromatin binding domain of Arabidopsis MBD7 methyl-CpG-binding domain, which allows MBD7 to bind to DNA independently of methylation state (Zemach et al., 2009, Exp Cell Res, 315:3554-62). When fused to other proteins, StkC can improve chromatin binding affinity, without compromising their ability to bind native target sites. In some particular embodiments, the StkC domain used by the present invention is residues 232-305 from MBD7. More specifically, in some embodiments, such domain comprises amino acid sequence as denoted by SEQ ID NO. 132.

It should be appreciated that in some specific embodiments, the present disclosure further encompasses the option of using any one of the DBP listed herein also as a DAD.

Still further it must be understood that the use of DBP in the disclosed HDR enhanced nucleic acid guided genome effector or modifier proteins, that is also PAM reduced and/or PAM abolished, further enhances gene editing efficiency and may further increase HDR. As indicated above, the Cas protein used for the nucleic acid guided genome modifier chimeric or fusion protein of the present disclosure, may be Cas protein devoid of, or having reduced nucleolytic activity. In some further embodiments, the Cas protein suitable for the nucleic acid guided genome modifier chimeric protein of the invention may be a Cas mutant or variant, that may further comprise at least one of: (a) at least one point mutation substituting aspartic acid residue at position 10 to alanine (D10A) and at least one point mutation substituting histidine residue 849 to alanine (H849A); and (b) at least one deletion of at least one of: (i) the HNH-nuclease domain or any fragment thereof; (ii) the REC2 domain or any fragments thereof; (iii) the FLEX domain or any fragments thereof; (iv) the RUVC domain or any fragments thereof; and (v) any combinations of (i), (ii), (iii), and (iv).

“Mutant” or “variant” as used herein, refers to the Cas protein used for the HDR enhanced nucleic acid guided genome modifier chimeric or fusion protein of the present disclosure, encoded by a sequence comprising at least one mutation, or in which at least a portion of the functionality of the sequence has been lost, or changed. As used herein, the term “mutation,” refers to any change in a nucleic acid sequence that may arise from at least one of, a deletion, addition, substitution, or rearrangement of at least one nucleotide in the mutated sequence. The mutation may also affect one or more properties of the proteins and/or steps that the sequence is involved in. For example, a change in a DNA sequence may lead to the synthesis of an altered mRNA and/or a protein that is active, partially active, inactive, or displaying at least one altered property, specificity, stability, bioavailability, solubility, size and the like.

As indicated above, the Cas protein used for the HDR enhanced nucleic acid guided genome modifier chimeric or fusion protein of the present disclosure, is in some embodiments, a defective Cas. A defective enzyme (e.g., a defective mutant, variant or fragment) may relate to an enzyme that displays an activity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced activity of about 98% to about 100%, as compared to the wild type active nuclease. More specifically, an enzyme that displays an activity reduced in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, as compared to the wild type active nuclease.

In some embodiments, the CRISPR-dCas protein suitable for the nucleic acid guided genome modifier chimeric protein of the invention may be capable of binding at least one target recognition element. As used herein, a “target recognition element” is any moiety, for example a moiety composed of nucleic acid sequence that specifically recognizes and binds a target sequence. In some embodiments, the target recognition element in connection with the present disclosure may be referred to a guide moiety. In some embodiments, such guide may comprise a nucleic acid sequence, for example, an RNA or DNA. In such embodiments, the guide may be referred to as a specificity conferring nucleic acid (SCNA), or as a gRNA. Still further, the gRNA is an RNA molecule composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion or alternatively, annealing of the endogenous tracrRNA with a targeting sequence (also named crRNA), providing both scaffolding/binding ability for Cas9 nuclease and targeting specificity. Also referred to as “single guide RNA” or “sgRNA” or SCNA.

In some more specific embodiments, the at least one target recognition element may be at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids or combinations thereof.

As indicated above, the second component of the HDR enhanced nucleic acid guided genome modifier chimeric protein of the invention is a nucleic acid modifier or effector component.

In some embodiments, the at least one nucleic acid modifier component suitable for the nucleic acid guided genome modifier chimeric protein of the invention may be a protein-based modifier, a nucleic acid-based modifier or any combinations thereof. More specifically, the protein-based modifier may be at least one of a nuclease, a methyltransferase, a methylated DNA binding factor, a transcription factor, a transcription repressor, a chromatin remodeling factor, a polymerase, a demethylase, an acetylase, a deacetylase, a kinase, a phosphatase, an integrase, a recombinase, a ligase, a topoisomerase, a gyrase, a helicase, and any combinations thereof.

In some embodiments, “the nucleic acid modifier or effector” component may be any component, element or specifically protein, polypeptide or nucleic acid sequence or oligonucleotide that upon direct or indirect interaction with a target nucleic acid sequence, modify or modulate the structure, function (e.g., expression), or stability thereof. Such modification may include the modification of at least one functional group, addition or deletion of at least one chemical group by modifying an existing functional group or introducing a new one such as methyl group. The modifications may include cleavage, methylation, demethylation, deamination and the like. Specific modifier component applicable in the present invention may include but are not limited to a protein-based modifier, for example, a nuclease, a methyltransferase, a methylated DNA binding factor, a transcription factor, transcription repressor, a chromatin remodeling factor, a polymerase, a demethylase, an acetylase, a deacetylase, a kinase, a phosphatase, an integrase, a recombinase, a ligase, a topoisomerase, a gyrase, a helicase, any combinations thereof or any fusion proteins comprising at least one of the modifier proteins disclosed by the invention.

In some specific embodiments, the nucleic acid modifier component may be at least one nuclease.

More specifically, as used herein, the term “nuclease” refers to an enzyme that in some embodiments display a nucleolytic activity, specifically, capable of cleaving the phosphodiester bonds between monomers of nucleic acids (e.g., DNA and/or RNA). Nucleases variously effect single and double stranded breaks in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. The nucleases belong just like phosphodiesterase, lipase and phosphatase to the esterases, a subgroup of the hydrolases. This subgroup includes the Exonucleases which are enzymes that work by cleaving nucleotides one at a time from the end (exo) of a polynucleotide chain. A hydrolyzing reaction that breaks phosphodiester bonds at either the 3′ or the 5′ end occurs. Eukaryotes and prokaryotes have three types of exonucleases involved in the normal turnover of mRNA: 5′ to 3′ exonuclease (Xrn1), which is a dependent decapping protein; 3′ to 5′ exonuclease, an independent protein; and poly (A)-specific 3′ to 5′ exonuclease. Members of this family include Exodeoxyribonucleases producing 5′-phosphomonoesters, Exoribonucleases producing 5′-phosphomonoesters, Exoribonucleases producing 3′-phosphomonoesters and Exonucleases active with either ribo- or deoxy-. Members of this family include exonuclease, II, III, IV, V, VI, VII, and VIII. As noted above, Endonucleases are enzymes that cleave the phosphodiester bond within a polynucleotide chain. Some endonucleases, such as deoxyribonuclease I, cut DNA relatively nonspecifically (without regard to sequence), while many, typically called restriction endonucleases or restriction enzymes, cleave only at very specific nucleotide sequences.

In some embodiment, the nuclease may be an active enzyme having a nucleolytic activity as specified above.

A restriction enzyme is an embodiment for endonuclease that cleaves DNA into fragments at or near its specific recognition sites within the molecule. To cut DNA, most restriction enzymes make two incisions, through each sugar-phosphate backbone (i.e., each strand) of the DNA double helix.

In some embodiments, Type IIS restriction enzymes recognize asymmetric DNA sequences and cleave outside of their recognition sequence, which can be removed, and can thus be used. Non-limiting examples of such restriction enzymes may include, but are not limited to FokI, AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, HgaI, HphI, HpyAV, MboII, MlyI, MmeI, MnlI, NmeAIII, PleI, SapI, SfaNI, and I-TEVI.

In some specific embodiments, the nuclease used as the effector/modifier component in the HDR enhanced nucleic acid guided genome modifier chimeric or fusion protein of the present disclosure may be at least one typeIIS nuclease or any cleavage domains thereof. These may include cleavage domains from Type IIS restriction endonucleases: AarI, Acc36I, AceIII, AclWI, AcuI, AjuI, AloI, AlwI, Alw26I, AmaCSI, ApyPI, AquII, AquIII, AquIV, ArsI, AsuHPI, BaeI, BarI, Bbr7I, BbsI, BbvI, BbvII, Bbv16II, BccI, BccI, Bce83I, BceAI, BceSIII, BceSIV, BcefI, BcgI, BciVI, Bco5I, Bco116I, BcoDI, BcoKI, BfiI, BfuI, BfuAI, BinI, Bli736I, Bme585I, BmrI, BmsI, BmuI, BpiI, BpmI, BpuAI, BpuEI, BpuSI, BsaI, BsaXI, BsbI, Bsc91I, BscAI, BseKI, BseMI, BseMII, BseRI, BseXI, BseZI, BsgI, BslFI, BsmAI, BsmBI, BsmFI, Bso311, BsoMAI, Bsp4231, BspCNI, BspD6I, BspIS4I, BspKT5I, BspLU11III, BspQI, BspST5I, BspTNI, BspTS514I, Bst6I, Bst12I, Bst19I, Bst71I, BstBS32I, BstFZ438I, BstGZ53I, BstH9I, BstMAI, BstOZ616I, Bst31TI, BstTS5I, BstV1I, BstV2I, BsuI, Bsu6I, Bsu537I, BtgZI, BtsI, BtsIMutI, BtsCI, BveI, Bve1B23I, CatHI, CchII, CchIII, Cco14983III, CdpI, CjeI, CjeF38011III, CjeIAIII, CjeNII, CjeNIII, CjePI, CjeP659IV, CjeYH002IV, CjuII, CseI, CspCI, CstMI, DraRI, DrdIV, EacI, Eam1104I, EarI, EciI, Eco31I, Eco57I, FaqI, FauI, Fph2801I, GeoICI, GsuI, HgaI, Hin4I, Hin4II, HphI, Hpy99XXII, HpyAV, HpyC1I, Ksp632I, LguI, Lsp1109I, LweI, MaqI, MboII, Mcr10I, MlyI, MmeI, MnlI, NcuI, NgoAVII, NgoAVIII, NlaCI, NmeAIII, NmeA6CIII, PciSI, PcoI, Phal, PlaDI, PleI, PpiI, PpsI, PspOMII, PspPRI, PsrI, RceI, RdeGBII, RlaII, RleAI, RpaI, RpaBI, RpaB5I, Rtr1953I, SapI, SchI, SdeAI, SdeOSI, SfaNI, SmuI, SspD5I, SstE37I, Sth132I, StsI, TaqII, TaqIII, TsoI, TspDTI, TspGWI, TstI, Tth111II, TthHB27I, UbaF9I, UbaF11I, UbaF12I, UbaF13I, UbaF14I, Vga4394211, VpaK32I, WviI.

In some more specific embodiments, the nuclease used in the HDR enhanced nucleic acid guided genome modifier chimeric or fusion protein of the present disclosure, may be a Type IIS restriction endonuclease or any fragment, variant, mutant, fusion protein or conjugate thereof.

In some further embodiments, the Type IIS restriction endonuclease may be FokI or any fragment, variant, mutant, fusion protein or conjugate thereof.

The enzyme FokI (Fok-1), naturally found in Flavobacterium okeanokoites, is a bacterial type IIS restriction endonuclease consisting of an N-terminal DNA-binding domain and a non-specific DNA cleavage domain at the C-terminal. Once the protein is bound to duplex DNA via its DNA-binding domain at the 5′-GGATG-3′ recognition site, the DNA cleavage domain is activated through dimerization and cleaves, without further sequence specificity, the first strand 9 nucleotides downstream and the second strand 13 nucleotides upstream of the nearest nucleotide of the recognition site leaving a typical 4 base overhang. DNA cleavage is mediated through the non-specific cleavage domain which also includes the dimerization surface. The dimer interface is formed by the parallel helices a4 and a5 and two loops P1 and P2 of the cleavage domain. The FokI cleavage domain's molecular mass is 21.8 kDa, being composed of 194 amino acids. In some embodiments, FokI may comprise the amino acid sequence as denoted by SEQ ID NO: 133, or any fragments, derivatives and variants thereof. In yet some further embodiments, a FokI variant useful in the present invention may comprise ancestral mutations. In some specific embodiments such FokI variant may comprise the amino acid sequence as denoted by SEQ ID NO. 134 (also referred to herein in the text and the figures as “ancestral FokI”, or as “consensus FokI”. In yet some further embodiments, a FokI variant may comprise the amino acid sequence as denoted by SEQ ID NO. 135 (also referred to herein as “enhanced FokI”). It should be appreciated that the present disclosure further encompasses any variations of the specified FokI variants.

In some further alternative or additional embodiments, the Type IIS restriction endonuclease used for the HDR enhanced nucleic acid guided genome modifier chimeric or fusion protein of the present disclosure, may be MmeI or any fragment, variant, mutant, fusion protein or conjugate thereof.

In some further embodiments, the Type IIS restriction endonuclease used for the HDR enhanced nucleic acid guided genome modifier chimeric or fusion protein of the present disclosure, may be MnlI or any fragment, variant, mutant, fusion protein or conjugate thereof.

In some further embodiments, the Type IIS restriction endonuclease HDR enhanced nucleic acid guided genome modifier chimeric or fusion protein of the present disclosure, may be BfiI or any fragment, variant, mutant, fusion protein or conjugate thereof.

Still further, it should be understood that in some further embodiments, the disclosed HDR enhanced nucleic acid guided genome modifier chimeric protein of the invention may further comprise additional structural and/or functional elements, that may improve the stability, bioavailability, affinity, activity and/or specificity thereof. For example, the HDR enhanced nucleic acid guided genome modifier chimeric or fusion protein of the present disclosure may further comprise at least one cellular localization domain such as Nuclear localization signal (NLS), at least one Mitochondrial leader sequence (MLS), for example, at least one Chloroplast leader sequence; and/or any sequences designed to transport or lead or localize a protein to a nucleic acid containing organelle, a cellular compartment or any subdivision of a cell.

According to some embodiments, a “cellular localization domain” which can localize the nucleic acid guided genome modifier chimeric protein of the invention or a system comprising the modifier/effector chimeric protein and at least one target recognition element, or any complex thereof, to a specific cellular or sub cellular localization in a living cell, may optionally be part of the modifier/effector component of the nucleic acid guided genome modifier chimeric protein of the invention. The cellular localization domain may be constructed by fusing the amino-acid sequence of one of these components to amino-acids incorporating a domain comprising a Nuclear localization signal (NLS); a Mitochondrial leader sequence (MLS); a Chloroplast leader sequence; and/or any sequences designed to transport or lead or localize a protein to a nucleic acid containing organelle, a cellular compartment or any subdivision of a cell. In some exemplary embodiments, the organism is eukaryotic, and the cellular localization domain comprises a nuclear localization domain (NLS) which allows the protein access to the nucleus and the genomic DNA within.

In some specific embodiments, the HDR enhanced nucleic acid guided genome modifier chimeric or fusion protein of the present disclosure is a chimeric protein. It should be appreciated that the HDR enhanced nucleic acid guided genome modifier chimeric or fusion protein of the present disclosure may be also referred to as the “chimeric protein of the present disclosure”, and also as the “machine”, as “HDR enhanced nucleic acid guided genome modifier”, and the like. It should be understood that all are used interchangeably in the present disclosure. Still further, in some embodiments, the chimeric protein of the invention may be in some particular and non-limiting embodiments, any one of: dScCas9-FokI-ZFQ variant; dScCas9-FokI-Lam variant; dScCas9-FokI-Strep variant; dScCas9-FokI-Vir variant; dScCas9-FokI-BRCA2 variant; dScCas9-FokI-DSS1 variant; dScCas9-FokI-BRCA2-Strep variant; dScCas9-FokI-DSS1-Strep variant; dScCas9-FokI-BRCA2-virD2 variant; dScCas9-FokI-DSS1-virD2 variant; dCas9-BfiI variant, dCas9-MnlI variant; dCas9-MmeI variant; dCas9-FokI-RAD54ntd variant; dCasFok-BRCA2 3NLS variant; dCasFok-DSS1 3NLS variant; dCasFok-ZFQ 1NLS variant; dCasFok-Strep 1NLS variant; dCasFok, 2NLS, N-terminal BRCA2 variant; dCasFok, 2NLS, N-terminal BRCA2, 6His variant; dCasFok, 2NLS, N-terminal Streptavidin variant; dCasFok, 2NLS, N-terminal Streptavidin, 6His variant; dCasFok, 2NLS, N-terminal Pot variant; dCasFok, 2NLS, N-terminal Pot, 6His variant; dCasFok, 1NLS, N-terminal Streptavidin, C-terminal BRCA2 variant; dCasFok, 1NLS, N-terminal Pot, C-terminal BRCA2 variant; dCasFok, 1NLS, ancestral RuvC+REC1/2, N- and C-terminal BRCA2 variant; dCasFok, 2NLS variant, ancestral RuvC+REC1/2, N-terminal BRCA2 variant; dCasFok, 2NLS, ancestral RuvC+REC1/2, N-terminal BRCA2, 6His variant; ancestral dCas9-FokI-RAD52id variant; DSS1 peptide (n-term), ancestral dCasFok variant; BRCA2 peptide 2 (N-term), ancestral dCasFok variant; RAD52 peptide (n-term), ancestral dCasFok variant; Streptavidin (n-term), ancestral dCasFok variant; BRCA2 peptide (c-term), ancestral dCasFok variant; DSS1 peptide (c-term), ancestral dCasFok variant; BRCA2 peptide 2 (c-term), ancestral dCasFok variant; RAD54 peptide (c-term), ancestral dCasFok; RAD52 peptide (c-term), ancestral dCasFok variant; Mdm2 peptide (c-term), ancestral dCasFok variant; Streptavidin (c-term), ancestral dCasFok variant; BRCA2 peptide (n-term), ancestral dCasFok, his-tagged variant; DSS1 peptide (n-term), ancestral dCasFok, his-tagged variant; BRCA2 peptide 2 (n-term), ancestral dCasFok, his-tagged variant; RAD54 peptide (n-term), ancestral dCasFok, his-tagged variant; RAD52 peptide (n-term), ancestral dCasFok, his-tagged variant; Streptavidin (n-term), ancestral dCasFok, his-tagged variant; BRCA2 peptide (n and c-term), ancestral dCasFok variant; BRCA2 peptide (n-term), DSS1 peptide (c-term), ancestral dCasFok variant; BRCA2 peptide (n-term), BRCA2 peptide 2 (c-term), ancestral dCasFok variant; BRCA2 peptide (n-term), RAD52 peptide (c-term), ancestral dCasFok variant; BRCA2 peptide (n-term), Mdm2 peptide (c-term), ancestral dCasFok variants; BRCA2 peptide (n-term), Streptavidin (c-term), ancestral dCasFok variant; DSS1 peptide (n-term), BRCA2 peptide (c-term), ancestral dCasFok variant; DSS1 peptide (n-term), BRCA2 peptide 2 (c-term), ancestral dCasFok variant; DSS1 peptide (n-term), RAD54 peptide (c-term), ancestral dCasFok variant; DSS1 peptide (n-term), Streptavidin (c-term), ancestral dCasFok variant; Streptavidin (n-term), BRCA2 peptide (c-term), ancestral dCasFok variant; Streptavidin (n-term), DSS1 peptide (c-term), ancestral dCasFok variant; Streptavidin (n-term), BRCA2 peptide 2 (c-term), ancestral dCasFok variant; Streptavidin (n-term), RAD52 peptide (c-term), ancestral dCasFok variant; and ancestral dCasFok, his-tagged variant.

In some embodiments, dScCas9-FokI-ZFQ variant may comprise an amino acid sequence as denoted by SEQ ID NO. 9.

In some embodiments, dScCas9-FokI-Lam variant (dScCas9-FokI fused to Lambda repressor DNA binding domain) may comprise an amino acid sequence as denoted by SEQ ID NO. 10.

In some embodiments, dScCas9-FokI-Strep variant (dScCas9-FokI fused to monomeric streptavidin) may comprise an amino acid sequence as denoted by SEQ ID NO. 11.

In some embodiments, dScCas9-FokI-Vir variant (dScCas9-FokI fused to monomeric virD2) may comprise an amino acid sequence as denoted by SEQ ID NO. 12.

In some embodiments, dScCas9-FokI-BRCA2 variant may comprise an amino acid sequence as denoted by SEQ ID NO. 13. In some embodiments, dScCas9-FokI-DSS1 variant may comprise an amino acid sequence as denoted by SEQ ID NO. 14.

In some embodiments, dScCas9-FokI-BRCA2-Strep variant (dScCas9-FokI fused to a BRCA2 peptide and to a monomeric streptavidin) may comprise an amino acid sequence as denoted by SEQ ID NO. 15.

In some embodiments, dScCas9-FokI-DSS1-Strep variant (dScCas9-FokI fused to a DSS1 peptide and to a monomeric streptavidin), as denoted by SEQ ID NO. 16.

In some embodiments, dScCas9-FokI-BRCA2-virD2 variant (dScCas9-FokI fused to a BRCA2 peptide and to a monomeric virD2) may comprise an amino acid sequence as denoted by SEQ ID NO. 17.

In some embodiments, dScCas9-FokI-DSS1-virD2 variant (dScCas9-FokI fused to a DSS1 peptide and to a monomeric virD2) may comprise an amino acid sequence as denoted by SEQ ID NO. 18.

In some embodiments, dCas9-BfiI variant may comprise an amino acid sequence as denoted by SEQ ID NO: 38.

In some embodiments, dCas9-MnlI variant may comprise an amino acid sequence as denoted by SEQ ID NO:37.

In some embodiments, dCas9-MmeI variant may comprise an amino acid sequence as denoted by SEQ ID NO: 36.

In some embodiments, dCas9-FokI-RAD54ntd variant may comprise an amino acid sequence as denoted by SEQ ID NO: 29.

In some embodiments, ancestral dCas9-FokI-RAD54ntd variant (ancestral dCas9-FokI fused to a RAD54 N-terminal domain peptide) may comprise an amino acid sequence as denoted by SEQ ID NO: 31.

In some embodiments, dCasFok-BRCA2, 3NLS variant may comprise an amino acid sequence as denoted by SEQ ID NO: 20.

In some embodiments, dCasFok-DSS1, 3NLS variant may comprise an amino acid sequence as denoted by SEQ ID NO: 21.

In some embodiments, dCasFok-ZFQ, 1NLS variant may comprise an amino acid sequence as denoted by SEQ ID NO: 22.

In some embodiments, dCasFok-Strep, 1NLS variant may comprise an amino acid sequence as denoted by SEQ ID NO: 23.

In some embodiments, dCasFok, 2NLS, N-terminal BRCA2 variant may comprise an amino acid sequence as denoted by SEQ ID NO:45.

In some embodiments, dCasFok, 2NLS, N-terminal BRCA2, 6His variant may comprise an amino acid sequence as denoted by SEQ ID NO:46.

In some embodiments, dCasFok, 2NLS, N-terminal Streptavidin variant may comprise an amino acid sequence as denoted by SEQ ID NO:47.

In some embodiments, dCasFok, 2NLS, N-terminal Streptavidin, 6His variant may comprise an amino acid sequence as denoted by SEQ ID NO: 48.

In some embodiments, dCasFok, 2NLS, N-terminal Pot variant may comprise an amino acid sequence as denoted by SEQ ID NO:49.

In some embodiments, dCasFok, 2NLS, N-terminal Pot, 6His variant may comprise an amino acid sequence as denoted by SEQ ID NO:50.

In some embodiments, dCasFok, 1NLS, N-terminal Streptavidin, C-terminal BRCA2 variant may comprise an amino acid sequence as denoted by SEQ ID NO: 51.

In some embodiments, dCasFok, 1NLS, N-terminal Pot, C-terminal BRCA2 variant may comprise an amino acid sequence as denoted by SEQ ID NO:52.

In some embodiments, dCasFok, 1NLS, ancestral RuvC+REC1/2, N- and C-terminal BRCA2 variant may comprise an amino acid sequence as denoted by SEQ ID NO:53.

In some embodiments, dCasFok, 2NLS, ancestral RuvC+REC1/2, N-terminal BRCA2 variant may comprise an amino acid sequence as denoted by SEQ ID NO: 54.

In some embodiments, dCasFok, 2NLS, ancestral RuvC+REC1/2, N-terminal BRCA2, 6His variant may comprise an amino acid sequence as denoted by SEQ ID NO:55.

In some embodiments, ancestral dCas9-FokI-RAD52id variant may comprise an amino acid sequence as denoted by SEQ ID NO: 32; In some embodiments, DSS1 peptide (n-term), ancestral dCasFok variant may comprise an amino acid sequence as denoted by SEQ ID NO: 142; BRCA2 peptide 2 (N-term), ancestral dCasFok variant may comprise an amino acid sequence as denoted by SEQ ID NO: 143; In some embodiments, RAD52 peptide (n-term), ancestral dCasFok variant may comprise an amino acid sequence as denoted by SEQ ID NO: 144; In some embodiments, Streptavidin (n-term), ancestral dCasFok variant may comprise an amino acid sequence as denoted by SEQ ID NO: 145; In some embodiments, BRCA2 peptide (c-term), ancestral dCasFok variant may comprise an amino acid sequence as denoted by SEQ ID NO: 146; In some embodiments, DSS1 peptide (c-term), ancestral dCasFok variant may comprise an amino acid sequence as denoted by SEQ ID NO: 147; In some embodiments, BRCA2 peptide 2 (c-term), ancestral dCasFok variant may comprise an amino acid sequence as denoted by SEQ ID NO: 148; In some embodiments, RAD54 peptide (c-term), ancestral dCasFok variant may comprise an amino acid sequence as denoted by SEQ ID NO: 149; RAD52 peptide (c-term), ancestral dCasFok variant may comprise an amino acid sequence as denoted by SEQ ID NO: 150; In some embodiments, Mdm2 peptide (c-term), ancestral dCasFok variant may comprise an amino acid sequence as denoted by SEQ ID NO: 151; In some embodiments, Streptavidin (c-term), ancestral dCasFok variant may comprise an amino acid sequence as denoted by SEQ ID NO: 152; In some embodiments, BRCA2 peptide (n-term), ancestral dCasFok, his-tagged variant may comprise an amino acid sequence as denoted by SEQ ID NO: 153; In some embodiments, DSS1 peptide (n-term), ancestral dCasFok, his-tagged variant may comprise an amino acid sequence as denoted by SEQ ID NO: 154; In some embodiments, BRCA2 peptide 2 (n-term), ancestral dCasFok, his-tagged variant may comprise an amino acid sequence as denoted by SEQ ID NO: 155; In some embodiments, RAD54 peptide (n-term), ancestral dCasFok, his-tagged variant may comprise an amino acid sequence as denoted by SEQ ID NO: 156; In some embodiments, RAD52 peptide (n-term), ancestral dCasFok, his-tagged variant may comprise an amino acid sequence as denoted by SEQ ID NO: 157; In some embodiments, Streptavidin (n-term), ancestral dCasFok, his-tagged variant may comprise an amino acid sequence as denoted by SEQ ID NO: 158; In some embodiments, BRCA2 peptide (n and c-term), ancestral dCasFok variant may comprise an amino acid sequence as denoted by SEQ ID NO: 159; BRCA2 peptide (n-term), DSS1 peptide (c-term), ancestral dCasFok variant may comprise an amino acid sequence as denoted by SEQ ID NO: 160; In some embodiments, BRCA2 peptide (n-term), BRCA2 peptide 2 (c-term), ancestral dCasFok variant may comprise an amino acid sequence as denoted by SEQ ID NO: 161; In some embodiments, BRCA2 peptide (n-term), RAD52 peptide (c-term), ancestral dCasFok variant may comprise an amino acid sequence as denoted by SEQ ID NO: 162; In some embodiments, BRCA2 peptide (n-term), Mdm2 peptide (c-term), ancestral dCasFok variants may comprise an amino acid sequence as denoted by SEQ ID NO: 163; In some embodiments, BRCA2 peptide (n-term), Streptavidin (c-term), ancestral dCasFok variant may comprise an amino acid sequence as denoted by SEQ ID NO: 164; In some embodiments, DSS1 peptide (n-term), BRCA2 peptide (c-term), ancestral dCasFok variant may comprise an amino acid sequence as denoted by SEQ ID NO: 165; In some embodiments, DSS1 peptide (n-term), BRCA2 peptide 2 (c-term), ancestral dCasFok variant may comprise an amino acid sequence as denoted by SEQ ID NO: 166; In some embodiments, DSS1 peptide (n-term), RAD54 peptide (c-term), ancestral dCasFok variant may comprise an amino acid sequence as denoted by SEQ ID NO: 167; DSS1 peptide (n-term), Streptavidin (c-term), ancestral dCasFok variant may comprise an amino acid sequence as denoted by SEQ ID NO: 168; In some embodiments, Streptavidin (n-term), BRCA2 peptide (c-term), ancestral dCasFok variant may comprise an amino acid sequence as denoted by SEQ ID NO: 169; In some embodiments, Streptavidin (n-term), DSS1 peptide (c-term), ancestral dCasFok variant may comprise an amino acid sequence as denoted by SEQ ID NO: 170; Streptavidin (n-term), BRCA2 peptide 2 (c-term), ancestral dCasFok variant may comprise an amino acid sequence as denoted by SEQ ID NO: 171; In some embodiments, Streptavidin (n-term), RAD52 peptide (c-term), ancestral dCasFok variant may comprise an amino acid sequence as denoted by SEQ ID NO: 172; and In some embodiments, ancestral dCasFok, his-tagged variant may comprise an amino acid sequence as denoted by SEQ ID NO: 173.

In some particular and non-limiting embodiments, of particular relevance is the machine comprising Streptavidin (n-term), RAD52 peptide (c-term), ancestral dCasFok, as denoted by SEQ ID NO: 172 and any variants and derivatives thereof, that display a clear enhanced HDR, that in some embodiments is synergistic, as also shown by FIG. 3E. Further particular machines in accordance with the present disclosure is the machine designated 15192 (SEQ ID NO: 171), that as shown by FIG. 3E also demonstrated enhanced HDR.

The disclosed HDR enhanced nucleic acid guided genome modifier chimeric or fusion protein is a polypeptide comprising an amino acid sequence. It should be noted that “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that in some embodiments may undergo post-translational modifications, such as phosphorylation, acetylation, glycosylation, manosylation, amidation, carboxylation, sulfhydryl bond formation, cleavage and the like.

By “fragments or peptides” it is meant a fraction of the protein of the invention. A “fragment” of a molecule, such as any of the amino acid sequences of the present invention, is meant to refer to any amino acid subset. This may also include “variants” or “derivatives” thereof. A “peptide” is meant to refer to a particular amino acid subset having a functional, structural activity or function displayed by the protein disclosed by the invention.

It should be appreciated that the present disclosure encompasses any variant or derivative of the HDR enhanced nucleic acid guided genome modifier chimeric or fusion protein disclosed herein, and any polypeptides that are substantially identical or homologue. The term “derivative” is used to define amino acid sequences (polypeptide), with any insertions, deletions, substitutions and modifications to the amino acid sequences (polypeptide) that either do not alter the activity of the original polypeptides or alter it purposefully. In this connection, a derivative or fragment of the variant of the invention may be any derivative or fragment of the variant and/or mutated molecule, specifically as denoted by SEQ ID NO: 9-18, 20-23, 31, 36-38, 45-55, 142-172, that do not reduce or alter the activity of the variant of the invention.

By the term “derivative” it is also referred to homologues, variants and analogues thereof. Proteins orthologs or homologues having a sequence homology or identity to the proteins of interest in accordance with the invention, specifically that may share at least 50%, at least 60% and specifically 65%, 70%, 75%, 80%, 85%, 90%, 91%, 92%, 93%, 94%, 95%, 96%, 97%, 98%, 99%, 100%, specifically as compared to the entire sequence of the proteins of interest in accordance with the invention, for example, any of the proteins that comprise the amino acid sequence as denoted by SEQ ID NO: 9-18, 20-23, 29, 31, 36-38, 45-55. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO: 9-18, 20-23, 31, 36-38, 45-55, 142-172.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as well as “substituted, “deleted”, “inserted”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, add or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention.

In a further aspect, the invention relates to a nucleic acid molecule comprising a nucleic acid sequence encoding at least one nucleic acid guided genome modifier chimeric protein having enhanced homology-directed repair or any variant, mutant, fusion/chimeric protein, complex or conjugate thereof. It should be noted that the present aspect further encompasses any construct, cassette, delivery vehicle and vector/s comprising the disclosed nucleic acid sequence. More specifically, the nucleic acid guided genome modifier chimeric protein of the invention may comprise: (a) at least one CRISPR-dCas protein devoid of a nucleolytic activity or any variant or mutant thereof; and (b) at least one nucleic acid modifier component. The nucleic acid guided genome modifier chimeric or fusion protein encoded by the nucleic acid sequence of the present disclosure further comprises at least one of: (c) at least one donor attachment domain (DAD) for binding at least one Donor nucleic acid molecule; and/or (d) at least one repair factor recruitment domain (RFRD).

In some embodiments, the nucleic acid guided genome modifier chimeric protein, complex or conjugate encoded by the nucleic acid sequence of the present disclosure, may be any one of the “machines” or “chimeras”, or HDR enhanced nucleic acid guided genome modifier chimeric or fusion protein of the present disclosure as defined above. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art.

It should be noted that the nucleic acid molecules (or polynucleotides) according to the present disclosure can be produced synthetically, or by recombinant DNA technology. Methods for producing nucleic acid molecules are well known in the art.

The nucleic acid molecule according to the present disclosure may be of a variable nucleotide length. For example, in some embodiments, the nucleic acid molecule according to the invention comprises 1-100 nucleotides, e.g., about 10, 20, 30, 40, 50, 60, 70, 80, 90 or 100 nucleotides. In other embodiments the nucleic acid molecule according to the invention comprises 100-1,000 nucleotides, e.g., about 100, 200, 300, 400, 500, 600, 700, 800, 900 or 1000 nucleotides. In further embodiments the nucleic acid molecule according to the invention comprises 1,000-10,000 nucleotides, e.g., about 1,000, 2,000, 3,000, 4,000, 5,000, 6,000, 7,000, 8,000, 9,000 or 10,000 nucleotides. In yet further embodiments the nucleic acid molecule according to the invention comprises more than 10,000 nucleotides, for example, 20,000, 30,000, 40,000, 50,000, 60,000, 70,000, 80,000, 90,000 or 100,000 nucleotides.

The invention further encompasses in some embodiments thereof at least one nucleic acid cassette comprising the nucleic acid sequence of the invention, or any vector or vehicle thereof. More specifically, the nucleic acid molecules provided by the invention may be comprised in some embodiments, within nucleic acid cassettes. The term “nucleic acid cassette” refers to a polynucleotide sequence comprising at least one regulatory sequence operably linked to a sequence encoding a nucleic acid sequence of interest. All elements comprised within the cassette of the invention are operably linked together. The term “operably linked”, as used in reference to a regulatory sequence and a structural nucleotide sequence, means that the nucleic acid sequences are linked in a manner that enables regulated expression of the linked structural nucleotide sequence.

Still further, the nucleic acid molecules of the invention or any cassettes thereof may be comprised within vector/s. Vector/s, as used herein, are nucleic acid molecules of particular sequence that can be introduced into a host cell, thereby producing a transformed host cell or be transiently expressed in the cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, (as detailed below) useful for transferring nucleic acids into target cells may be applicable in the present invention. The vectors comprising the nucleic acid(s) may be maintained episomally, e.g., as plasmids, minicircle DNAs, viruses such cytomegalovirus, adenovirus, or they may be integrated into the target cell genome, through homologous recombination or random integration, e.g., retrovirus-derived vectors such as AAV, MMLV, HIV-1, ALV, etc.

As indicated above, in some embodiments, viral vectors may be applicable in the present invention. The term “viral vector” refers to a replication competent or replication-deficient viral particle which are capable of transferring nucleic acid molecules into a host.

In some embodiments such viral vectors may be used for transient expression of the nucleic acid sequence encoding the nucleic acid guided genome modifier chimeric or fusion protein of the present disclosure, or any systems thereof, in the cell and may or may not be present in the cells ultimately delivered to the subject or any other organism. The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e., vectors constructed using complementary coding sequences from more than one viral subtype. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

More specifically, in some embodiments, the nucleic acid molecules encoding the nucleic acid guided genome modifier chimeric or fusion protein of the present disclosure, or any systems thereof, suitable in the methods of the present disclosure may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express genes of interest for months in quiescent cells. Production systems for rAAV vectors typically consist of a DNA-based vector containing a transgene expression cassette, which is flanked by inverted terminal repeats (payload). Construct sizes are limited to approximately 4.7-5.0 kb, which corresponds to the length of the wild-type AAV genome. In some embodiments it would thus be advantageous to have a payload smaller than this upper limit. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of genes of interest when rAAV is used as gene delivery tool. Indeed, significant differences in transduction efficacy of various serotypes for particular tissues and cell types have been described. Thus, in some embodiments AAV serotype 6 may be suitable for the methods of the invention. In yet some further embodiments, AAV serotype 8 may be suitable for the methods, systems, and the nucleic acid guided genome modifier chimeric protein of the invention.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB but leads to significantly shortened onsets of expression in comparison with conventional single-stranded AAV expression constructs (ssAAV). Thus, in some embodiments, ssAAV may be applicable as a viral vector by the methods of the invention.

In yet some further embodiments, HDAd vectors may be suitable for the methods, systems, and the nucleic acid guided genome modifier chimeric protein of the invention. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long-term expression of the transgene.

Still further, in some embodiments, SV40 may be used as a suitable vector by the methods, systems, and the nucleic acid guided genome modifier chimeric protein of the invention. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10(12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome.

In certain embodiments, an appropriate vector that may be used by the invention may be a retroviral vector. A retroviral vector consists of proviral sequences that can accommodate the gene of interest, to allow incorporation of both into the target cells. The vector may also contain viral and cellular gene promoters, to enhance expression of the gene of interest in the target cells. Retroviral vectors stably integrate into the dividing target cell genome so that the introduced gene is passed on and expressed in all daughter cells. They contain a reverse transcriptase that allows integration into the host genome. In yet some alternative embodiments, lentiviral vectors may be used in the present invention. Lentiviral vectors are derived from lentiviruses which are a subclass of Retroviruses. Commonly used retroviral vectors are “defective”, i.e., unable to produce viral proteins required for productive infection. Rather, replication of the vector requires growth in a packaging cell line. To generate viral particles comprising the nucleic acids sequence of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid molecules of the invention that contains the nucleic acids sequence of interest into packaging cell lines and of collecting the viral particles that are generated by the packaging lines are well known in the art.

In some alternative embodiments, the vector may be a non-viral vector. More specifically, such vector may be in some embodiments any one of plasmid, minicircle and linear DNA, ssDNA (that are especially useful for donor integration at cleavage site) or RNA (useful to avoid long term expression and or integration) or a modified polynucleotide (mainly chemically protective modifications to protect RNA or DNA-RNA chimeras to enhance specificity and or stability).

Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is sometimes less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA, RNA or RNP entrance into the targeted cells is facilitated.

In more specific embodiments, the vector may be a naked DNA vector. More specifically, such vector may be for example, a plasmid, minicircle or linear DNA.

Naked DNA alone may facilitate transfer of a nucleic acid sequence (2-200 Kb or more) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria.

Linear DNA or Doggybone™ are double-stranded, linear DNA construct that solely encodes a payload expression cassette, comprising antigen, promoter, polyA tail and telomeric ends.

It should be appreciated that all DNA vectors disclosed herein, may be also applicable for the methods, systems and compositions of the invention.

Still further, it must be appreciated that the invention further provides any vectors or vehicles that comprise any of the nucleic acid molecules disclosed by the invention, as well as any host cell expressing the nucleic acid molecules disclosed by the invention.

It should be understood that any of the viral vectors disclosed herein may be relevant to any of the nucleic acid molecules discussed in other aspects of the invention, specifically to nucleic acid molecules encoding the SCNA (gRNA), the Donor or the protein components as described by the invention.

As indicated above, vectors may be provided directly to the subject cells thereby being contacted with the cell/s. In other words, the cells are contacted with vectors comprising the nucleic acid molecules of the invention that comprise the nucleic acid sequence of interest such that the vectors are taken up by the cells. Methods for contacting cells with nucleic acid vectors that are plasmids, such as electroporation, calcium chloride transfection, and lipofection (e.g. using Lipofectamin), are well known in the art. DNA can be introduced as naked nucleic acid, as nucleic acid complexed with an agent such as a liposome, nanoparticles or poloxamer, or can be delivered by viruses (e.g., adenovirus, AAV).

In another aspect, the invention provides a nucleic acid guided genome modifier system having enhanced homology-directed repair. More specifically, the nucleic acid guided genome modifier system of the invention may comprise: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. It should be noted that the nucleic acid guided genome modifier chimeric or fusion protein of the disclosed system may comprise (i) at least one CRISPR-dCas protein devoid of a nucleolytic activity or any variant or mutant thereof; and (ii) at least one nucleic acid modifier component. The nucleic acid guided genome modifier chimeric or fusion protein of the disclosed system further comprises at least one of (iii) at least one DAD for binding at least one Donor nucleic acid molecule; and (iv) at least one RFRD. Specially, the system may further comprise at least one of: (b) at least one donor nucleic acid molecule; and (c) at least one target recognition element, or any nucleic acid sequence encoding the target recognition element.

In some embodiments, the DAD may at least one of: a sequence specific donor attachment domain, a non-sequence specific donor attachment domain and a covalent interaction domain.

In some specific embodiments, the DAD may be a sequence specific DAD comprising at least one of a zinc finger DNA binding domain, a lambda repressor DNA binding domain, a Gal4 DNA binding domain and a Pot1 ssDNA binding domain.

In some other embodiments, the DAD is a covalent interaction domain may comprise a virD2 domain.

In some other embodiments, the DAD may be a non-sequence specific donor attachment domain comprising at least one domain of an affinity pair, specifically, a streptavidin domain.

In another embodiments, the RFRD suitable for the system of the invention may recruit a protein involved in the HDR pathway of DSBs.

In some specific embodiments, the protein involved in HDR may be any one of a Recombination Protein A (Rad) family member, a Fanconi Anemia Core Complex member, Tumor Suppressor p53, or C-Terminal-Binding Protein-Interacting Protein (CtIP).

In some embodiments, the repair factor recruitment domain RFRD comprises at least one of: the BRCA2 protein, or any fragment or peptides thereof for recruitment of Rad51 and Rad52; the DSS1 protein, or any fragment or peptides thereof for recruitment of Rad52, the RAD52 protein, or any fragment or peptides thereof or the RAD54 protein, any fragment or peptides thereof.

In some embodiments, Cas protein suitable for the nucleic acid guided genome modifier chimeric protein, complex or conjugate of the systems of the present disclosure may be at least one of Cas9, CasX, Cas12a1, CasF, Cas14a1, an ancestral Cas and Cas14b5.

In yet other embodiments, CRISPR-dCas protein used for the HDR enhanced nucleic acid guided genome modifier chimeric or fusion protein of the present disclosure, may be a Cas protein having a reduced or abolished PAM constraint, and the at least one of the PAM Binding Domain (PDB) of the CRISPR-dCas protein suitable for the system of the invention, or any fragment of the PBD, and at least one amino acid residue adjacent to the PBD, may be deleted or replaced.

In some embodiments, the HDR enhanced nucleic acid guided genome modifier chimeric or fusion protein of the present disclosure of the system of the invention may be as defined as defined above with respect to the first aspect of the invention.

In some further embodiments, the CRISPR-dCas protein of the HDR enhanced nucleic acid guided genome modifier chimeric or fusion protein of the present disclosure, suitable for the system of the invention may be capable of binding at least one target recognition element. Moreover, in some embodiments, the systems disclosed herein, may comprise at least one target recognition element.

In some embodiments, at least one target recognition element is at least one nucleic acid target recognition element, said target recognition element is at least one of: a single strand RNA molecule, a double strand RNA molecule, a single strand DNA, a double strand DNA, a modified DNA molecule, a modified RNA molecule, a LNA, a PNA and any hybrid or combinations thereof. In some embodiments, the target recognition element of the disclosed system may comprise at least one gRNA and/or SCNA as defined by the present disclosure.

Still further, in some embodiments, the systems of the present disclosure may comprise in addition to the HDR enhanced nucleic acid guided genome modifier chimeric or fusion protein of the systems of the present disclosure, at least one donor nucleic acid molecule. In other embodiments, the donor nucleic acid molecule of the system of the invention may comprise at least one of nucleic acid sequence for incorporation into a target site within a target nucleic acid sequence.

“Donor nucleic acid” or “donor nucleic acid molecule” is defined here as any nucleic acid molecule that comprise at least one nucleic acid sequence supplied to a cell or receptacle to be inserted, incorporated or recombined wholly or partially into the target sequence within the nucleic acid molecules of the cell, either by DNA repair mechanisms, homologous recombination (HR), or by non-homologous end-joining (NHEJ). Such nucleic acid sequence may be referred to herein as a “replacement sequence”.

In some specific embodiments, the enhanced HDR nucleic acid guided genome modifier chimeric or fusion protein, systems, compositions and methods of the present disclosure uses at least one donor molecule for an HDR reaction. A Donor nucleic acid molecule may be composed of or comprise at least one nucleic acid sequence (either RNA or DNA or a modified nucleic acid or a combination thereof). Donor nucleic acid consisting of DNA or modified DNA may also be referred to as “donor DNA”. It should be noted that the donor molecule useful in the present disclosure may be either single strand DNA or double strand DNA molecule or comprise combinations of regions which are ssDNA and regions which are dsDNA, and moreover, may be prepared either recombinantly or synthetically.

In some embodiments, specifically when the donor nucleic acid molecule is incorporated into the target nucleic acid sequence via homologous recombination (HR), the donor nucleic acid sequence may also comprise, or specifically flanked by, at least one homology arm, that display complementarity to a nucleic acid sequence flanking the target site for incorporation.

In some optional embodiments, the donor nucleic acid molecule may be flanked by at least one homology arm. The term “flanked” as used herein refers to a nucleic acid sequence positioned between two defined regions. For example, as indicated above, the replacement-sequence is flanked by a first and a second, for example, left and right homology arms. Homology arms comprise sequences that display homology to the target site.

In some embodiments, the homology arm may display complementarity, at least partial complementarity, to sequences that flank the target site within a target nucleic acid sequence.

In another embodiments, the donor nucleic acid molecule may further comprise an attachment region that binds the DAD of the nucleic acid guided genome modifier chimeric protein, complex or conjugate disclosed herein.

Thus, in yet some further embodiments, the donor nucleic acid may comprise a recognition or attachment region, configured to associate/bind/attach with any nucleic acid guided genome modifier chimeric protein of the HDR enhanced nucleic acid guided genome modifier chimeric or fusion protein of the present disclosure. In some specific embodiments, such HDR enhanced nucleic acid guided genome modifier chimeric protein may comprise a PAM reduced/free Cas protein or any chimera, complex or conjugate thereof. In some embodiments, the recognition/attachment region may be located at the 5′-end and/or the 3′-end of the donor nucleic acid molecule. In some embodiments, the recognition or attachment region may comprise a chemical group or a modification that is recognized and bound by a chemical group comprised within the DAD, thereby forming a binding pair, for example Biotin-Avidin; Biotin-Streptavidin; Biotin-modified forms of Avidin; Protein-protein interactions; protein-nucleic acid interactions; ligand-receptor interactions; ligand-substrate interactions; antibody-antigen interactions; single chain antibody-antigen; antibody or single chain antibody-hapten interactions; hormone-hormone binding protein; receptor-agonist; receptor-receptor antagonist; anti-Fluorescein single-chain variable fragment antibody (anti-FAM ScFV)-Fluorescein; anti-DIG single-chain variable fragment (scFv) immunoglobin (DIG-ScFv)-Digoxigenin (DIG); IgG-protein A; enzyme-enzyme cofactor; enzyme-enzyme inhibitor; single-strand DNA-VirE2; StickyC-dsDNA; RISC-RNA; viral coat protein-nucleic acid and Agrobacterium VirD2-VirD2 binding protein; and any variants thereof. These proteins may be referred to, in some embodiments as “non-sequence specific donor attachment domains”.

In some embodiments, binding/association between the donor nucleic acid and the HDR enhanced nucleic acid guided genome modifier chimeric or fusion protein of the invention, complex or conjugate thereof is covalently created in vivo. In some embodiments, the covalent association of the HDR enhanced nucleic acid guided genome modifier chimeric protein of the invention or any chimera, complex or conjugate thereof and the donor nucleic acid molecule, results from a biological interaction of Agrobacterium VirD2-Right border sequence or any variants thereof and is created in a bacterium comprising Agrobacterium.

In some embodiments, the recognition region of the donor nucleic acid molecule comprises a nucleotide motif or sequence capable of interacting/attaching/binding with the DAD of the HDR enhanced nucleic acid guided genome modifier chimeric protein of the invention or any chimera, complex or conjugate thereof. In some embodiments, the interaction pair may include any one of Zinc finger protein-Zinc finger motif; restriction enzyme recognition domain-restriction enzyme recognition sequence; DNA binding domain of transcription factor-DNA motif; repressor-operator; Leucine zipper-promoter; Helix loop helix-E box domain; RNA binding motifs comprising Arginine-Rich Motif domains, αβ protein domains, RNA Recognition Motif (RRM) domains, MS2 coat protein-MS2 RNA binding hairpin, K-Homology Domains, Double Stranded RNA Binding Motifs, RNA-binding Zinc Fingers, and RNA-Targeting Enzymes-cognate specific RNA sequence; HIV-rev protein-Stem IIB of the HIV rev response element (RRE); Bovine immunodeficiency virus (BIV) Tat main binding domain-loop 1 of the BIV trans-acting response element (TAR) sequence; Phage lambda, phi21, and P22 Nproteins—The boxB loop hairpins in the N-utilization (nut) sites in their respective RNAs. These proteins may be referred to as “sequence specific donor attachment domains”.

Still further, in some particular embodiments, in addition to the sites or moieties that bind the DAD of the HDR enhanced nucleic acid guided genome modifier chimeric or fusion protein of the present disclosure, the donor molecules used in the systems of the present disclosure may also contain at least one binding site for at least one target recognition element (e.g., SCNA, gRNA and the like). Such at least one binding site/s (for example, those illustrated in FIG. 3A, (BS)), may flank the replacing nucleic acid sequence. Still further, in some embodiments, such at least one BS, may be flanked by at least one of the homology arm/s of the disclosed donor molecule (e.g., LHA, RHA). It should be noted that in such case, the target recognition element acts as the DAD that connects between the donor molecule via the BS, and the HDR enhanced nucleic acid guided genome modifier chimeric or fusion protein of the present disclosure.

In some embodiments, BS useful for attaching the target recognition element may comprise the nucleic acid sequence as denoted by SEQ ID NOs: 186 and 187.

In yet some other embodiments, the system of the invention may further comprise an agent to block cell cycle in G2/S phase. In some alternative embodiments, the system of the invention may further comprise small molecules to enhance HDR or reduce NHEJ such as chemical inhibition of factors repressing or chemical inhibition of NHEJ e.g., Ligase IV inhibition or DNA-PKcs inhibition. Non-limiting embodiments for HDR enhancing molecule/s useful in the present disclosure is the enhancer V2 (IDT), that is a small molecule that increases HDR efficiency in a broad range of cells.

In some further embodiments, the at least one nucleic acid sequence for incorporation of the donor nucleic acid molecule of the system of the invention may be a replacement sequence of a target nucleic acid of interest in the target site.

In some embodiments, such replacement sequence may comprise at least one nucleic acid sequence encoding a product (e.g., protein and/or RNA) that is directly or indirectly essential, beneficial or advantageous for the expressing target cell. In some embodiments, such replacement sequence may comprise the native, non-mutated version of a gene or any nucleic acid sequence that should replace the mutated version in the target cell. It should be however understood that the system of the invention further provides a tool that enables manipulation of genes or gene fragments that do not necessarily comprise any mutation. The replacement gene may be in some embodiment, a gene or fragment thereof that may comprise mutation or any manipulation that may improve and/or change the native nucleic acid sequence within the target cell, or even modulate the expression of a target nucleic acid sequence, e.g., at least one gene or any fragments thereof. In some embodiments, the length of such donor nucleic acid molecule, or specifically, replacement nucleic acid sequence may range between about 100,000 nucleotides or more, to about 10 nucleotides or less. More specifically, the length of the nucleic acid sequence of interest may be about 100,000 nucleotides in length, or less than 75,000 nucleotides in length or less than 50,000 nucleotides in length, or less than 40,000 nucleotides in length, or less than 30,000 nucleotides in length, or less than 20,000 nucleotides in length, or less than 15,000 nucleotides in length, or less than 10,000 nucleotides in length, or less than 5000 nucleotides in length, or less than 1000 nucleotides in length, or less than 900 nucleotides in length, or less than 800 nucleotides in length, or less than 700 nucleotides in length, or less than 600 nucleotides in length, or less than 500 nucleotides in length, or less than 450 nucleotides in length, or less than 400 nucleotides in length, or less than 300 nucleotides in length, or less than 200 nucleotides in length, or less than 100 nucleotides in length, or less than 50 nucleotides in length, or less than 40 nucleotides in length, or less than 30 nucleotides in length, or less than 20 nucleotides in length, or less than 10 nucleotides in length. In some embodiments, the replacement nucleic acid sequence may be in the length of 20,000 (20 Kb) nucleotides or more.

In some embodiments, the replacement sequence comprises a sequence that differs from the target nucleic acid sequence in at least 1, 2, 3, 4, 5, 6, 7, 8, 9, 10 or more, 20, 30, 40, 50, 60, 70, 80, 90, 100 or more, 200, 300, 400, 500 nucleotides or more. It should be understood that the replacement sequence differs from the target sequence that is replaced, and display in some embodiments only 50% to 99% identity, for example, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identity.

In another aspect, the invention provides at least one cell, and in some embodiments any host cell, or any population of cells comprising the cell in accordance with the invention. More specifically, the host cell of the invention or any populations thereof, may comprise and/or may be modified by, at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. The nucleic acid guided genome modifier chimeric or fusion protein comprised within, or modifying the cell of the present disclosure, may comprise (i) at least one defective CRISPR-Cas protein devoid of a nucleolytic activity or any variant or mutant thereof; and (ii) at least one nucleic acid modifier component. The nucleic acid guided genome modifier chimeric or fusion protein of the disclosed cell further comprises at least one of: (iii) at least one DAD for binding at least one Donor nucleic acid molecule; and/or (iv) at least one RFRD. The host cell may further comprise and/or modified by: (b) at least one donor nucleic acid molecule; (c) at least one target recognition element or any nucleic acid sequence encoding the target recognition element; (d) at least one nucleic acid cassette or any vector or vehicle comprising at least one of: (i) the nucleic acid sequence of (a); (ii) the nucleic acid sequence of (b); (iii) the nucleic acid sequence of (c); and (iv) the nucleic acid sequence of (a) and at least one of (b) and (c); and (e) at least one system comprising (a) and at least one of (b) and (c).

In some embodiments, Cas protein suitable for the nucleic acid guided genome modifier chimeric protein, complex or conjugate of the disclosed cells may be at least one of Cas9, CasX, Cas12a1, CasF, Cas14a1, an ancestral Cas and Cas14b5. In yet some further embodiments, the CRISPR-dCas protein of the nucleic acid guided genome modifier chimeric or fusion protein used herein to modify the disclosed cells has reduced or abolished PAM constraint. More specifically, in such PAM reduced or abolished protein, at least one of the PBD, any fragment of the PBD, and/or at least one amino acid residue adjacent to the PBD, is deleted or replaced.

In some specific embodiments, the nucleic acid guided genome modifier chimeric protein, complex or conjugate used to modify the disclosed host cell/s may be as defined above, in connection with other aspects of the present disclosure, the nucleic acid molecule of the host cell of the invention may be as defined above, in connection with other aspects of the present disclosure, and the system of the host cell of the invention may be as defined by above, in connection with other aspects of the present disclosure. In yet some further embodiments, it should be appreciated that any vector, construct, delivery vehicle that comprises the encoding nucleic acid sequences of the present disclosure, and specifically, those defined herein in connection with other aspects of the present disclosure, are applicable to the disclosed aspect as well.

The present disclosure provides modified, specifically, genetically modified cells that were manipulated by the HDR enhanced nucleic acid guided genome modifier chimeric or fusion protein of the present disclosure, and/or systems and compositions thereof.

The term modified cells as used herein, includes a cell into which a heterologous (e.g., exogenous) nucleic acid or protein (e.g., nucleic acid guided genome modifier chimeric or fusion protein of the present disclosure) or Ribonucleoprotein (RNP) thereof (e.g., the system of the invention), has been introduced. Moreover, the introduction of the nucleic acid sequence, for example, the sequence of the donor molecule, replaces target sequences, disrupt and/or adds nucleic acid sequence/s to the original sequence in the target cell. Persons of skill upon reading this disclosure will understand that such terms refer not only to the particular subject cell, but also is used to refer to the progeny of such a cell. Because certain modifications may occur in succeeding generations due to either mutation or environmental influences, developmental maturation, or due to the intended action of the invention, such progeny may not, in fact, be identical to the parent cell, but are still included within the scope of the term “host cell”. In some embodiments, the host cells provided by the invention are transduced or transfected by the nucleic acid sequences provided by the invention that encode the nucleic acid guided genome modifier chimeric or fusion protein of the present disclosure and systems thereof. This may refer in some embodiments, to cells that underwent a transfection procedure, meaning the introduction of a nucleic acid, e.g., an expression vector, or a replicating vector, into recipient cells by nucleic acid-mediated gene transfer. It should be appreciated that alternatively, or in combination with nucleic acids encoding components of the invention, all or part of the components may be delivered to the cell of the present disclosure as an RNA, as a protein, or as a preassembled RNP. Transfection of eukaryotic cells may be either transient or stable, and is accomplished by various ways known in the art.

For example, transfection of eukaryotic cells may be chemical, e.g., via a cationic polymer (such as DEAE-dextran, polyethyleneimine, dendrimer, polybrene, calcium), calcium phosphate (e.g., phosphate, lipofectin, DOTAP, lipofectamine, CTAB/DOPE, DOTMA) or via a cationic lipid. Transfection of eukaryotic cells may also be physical, e.g., via a direct injection (for example, by Micro-needle, AFM tip, Gene Gun), via biolistic particle delivery (for example, phototransfection, Magnetofection), or via electroporation (i.e., Lonza Nucleofector), laser-irradiation, sonoporation or a magnetic nanoparticle. Transfection of eukaryotic cells may also be biological (i.e., use of Agrobacterium in plants (or viruses or virus vectors also known as transduction).

Still further, the term “host cells”, or cells with respect to the modified (or genetically modified) cell of the present disclosure, as used herein refers to any cell known to a skilled person wherein the HDR enhanced nucleic acid guided genome modifier chimeric or fusion protein of the present disclosure, functional fragments or peptides thereof or any nucleic acid molecule or combination thereof according to the invention may be introduced. For example, a host cell may be any prokaryotic or eukaryotic cell of a unicellular or multi-cellular organism. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein.

The “host cell” as used herein refers also to cells which can be transformed or transfected with naked DNA, any plasmid or expression vectors constructed using recombinant DNA techniques. A drug resistance or other selectable marker carried on the transforming or transfecting plasmid is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the phenotype for survival.

Thus, the cell of the present disclosure is modified and/or genetically engineered by the nucleic acid guided genome modifier chimeric or fusion protein of the present disclosure, and any systems and compositions thereof, and any nucleic acid sequence encoding the same.

As indicated above, the disclosed cells and population of cells in some embodiments, relate to any eukaryotic cells. Eukaryotic cells may be mammalian cells, plant cells, fungi or cells of any organism. As used herein, the term “eukaryotic cell” refers to any cell type having a nucleus enclosed within a nuclear envelope, or any cell derived from such ceil (e.g., erythrocytes, Platelets and any anucleate cell). The cells are derived from any organism of the kingdom Eukaryota or Eukarya. Still further, it should be understood that the present disclosure further encompasses any eukaryotic cell known to a person skilled in the art which is suitable for genetic manipulation, or any cell nucleated or anucleate derived from such genetically manipulated cell. It should be noted that the term “eukaryotic cells” as used herein, further encompasses the autologous cells or allogeneic cells used by the methods of the invention via adoptive transfer, as discussed herein after in connection with other aspects of the invention. Thus, eukaryote cells as herein defined may be derived from animals, plants and fungi, for example, but not limited to, insect cells, yeast cells or mammalian cells.

It should be further understood that the term “Cell”, is defined here as to comprise any type of cell, prokaryotic or a eukaryotic cell, isolated or not, cultured or not, differentiated or not, and comprising also higher-level organizations of cells such as tissues, organs, calli, organisms or parts thereof. Exemplary cells include but are not limited to vertebrate cells, mammalian cells, human cells, plant cells, animal cells, invertebrate cells, nematodal cells, insect cells, stem cells, and the like. Other suitable cells applicable in the present disclosure, e.g., stem cells and the like, will be discussed and defined in connection with other aspects of the present disclosure, and are therefore encompassed in the present aspect as well.

In some specific embodiments, the at least one target recognition element of the host cell of the invention may be at least one of: a single strand RNA molecule, a double strand RNA molecule, a ssDNA, a dsDNA, a modified DNA molecule, a modified RNA molecule, a LNA, a PNA and any hybrid or combinations thereof.

In a further aspect, the invention provides a composition. Specifically, the composition of the invention may comprise at least one of: (a) a nucleic acid guided genome modifier chimeric or fusion protein, complex or conjugate having enhanced homology-directed repair, or at least one nucleic acid sequence encoding said nucleic acid guided genome modifier chimeric or fusion protein. The nucleic acid guided genome modifier chimeric or fusion protein of the disclosed composition may comprise: (i) at least one CRISPR-dCas protein devoid of a nucleolytic activity or any variant or mutant thereof; and (ii) at least one nucleic acid modifier component. The chimeric or fusion protein of the disclosed composition further comprises at least one of: (iii) at least one donor attachment domain (DAD) for binding a Donor nucleic acid molecule; and/or (iv) at least one repair factor recruitment domain (RFRD). The composition of the invention may comprise: (b) at least one donor nucleic acid molecule; (c) at least one target recognition element or any nucleic acid sequence encoding said target recognition element. Still further, in some embodiments, the disclosed compositions may comprise (d), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). In some further embodiments, the composition may comprise (e), at least one system comprising (a) and at least one of (b) and (c). In some further embodiments, the composition may comprise (f), at least one cell comprising and/or modified by at least one of: the nucleic acid cassette or any vector or vehicle of (d) and the at least one system of (e); or any matrix, nano- or micro-particle comprising at least one of (a), (b), (c), (d), (e) and (f). The disclosed composition optionally further comprises at least one of pharmaceutically acceptable carrier/s, diluent/s, excipient/s and additive/s.

In some embodiments, CRISPR-dCas protein suitable for the HDR enhanced nucleic acid guided genome modifier chimeric protein, complex or conjugate of the disclosed compositions may be at least one of Cas9, CasX, Cas12a1, CasF, Cas14a1, an ancestral Cas and Cas14b5. In yet some further embodiments, the CRISPR-dCas protein has reduced or abolished PAM constraint and the at least one of the PBD of the CRISPR-dCas protein suitable for the composition of the invention, any fragment of the PBD, and at least one amino acid residue adjacent to the PBD, may be deleted or replaced.

In other embodiments, the nucleic acid guided genome modifier chimeric protein, complex or conjugate of the composition of the invention may be as further defined above, in connection with other aspects of the present disclosure, the nucleic acid of the composition of the invention may be as further defined above, in connection with other aspects of the present disclosure, the system of the composition of the invention may be as further defined above, in connection with other aspects of the present disclosure, and the host cell of the composition of the invention may be as further defined above in connection with other aspects of the present disclosure.

In yet some further embodiments, the composition of the invention may optionally further comprise at least one of pharmaceutically acceptable carrier/s, excipient/s, additive/s diluent/s and adjuvant/s.

The pharmaceutical compositions of the invention can be administered and dosed by the methods of the invention, in accordance with good medical practice, systemically, for example by parenteral intravenous. It should be noted however that the present disclosure may further encompass additional administration modes. Thus, the disclosed compositions may be adapted for any appropriate administration mode, for example, any of the administration modes of the present disclosure. In other examples, the pharmaceutical composition can be introduced to a site by any suitable route including intraperitoneal, subcutaneous, transcutaneous, topical, intramuscular, intraarticular, subconjunctival, or mucosal, e.g., oral, intranasal, or intraocular administration.

Local administration to the area in need of treatment may be achieved by, for example, by local infusion during surgery, topical application, direct injection into the specific organ. More specifically, the compositions used in any of the methods of the invention, described herein before, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically the protein, nucleic acid, host cell of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question.

Still further, pharmaceutical preparations are compositions that include the protein, nucleic acid, host cell of the invention present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid or liquid such as tablets, capsules, powders, granules, ointments, solutions, suppositories, injections, inhalants, gels, microspheres, and aerosols. As such, administration of the protein, nucleic acid, host cell of the invention can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity, or it may be formulated for sustained release.

Still further, the composition/s of the invention and any components thereof may be applied as a single one-time dose, as a single daily dose or multiple daily doses, preferably, every 1 to 7 days. It is specifically contemplated that such application may be carried out once or several times in the lifetime of a patient, once, twice, thrice, four times, five times or six times daily, or may be performed once daily, once every 2 days, once every 3 days, once every 4 days, once every 5 days, once every 6 days, once every week, two weeks, three weeks, four weeks or even more than a month. The application of the nucleic acid guided genome modifier chimeric or fusion protein of the present disclosure, systems thereof, nucleic acid sequences or any vectors thereof, host cell/s transformed or transfected, and/or modified, and/or comprising by the nucleic acid sequence, in accordance with the invention or of any component thereof, or the effects thereof, may last up to the lifetime of the patient, a day, two days, three days, four days, five days, six days, a week, two weeks, three weeks, four weeks, a month, two months three months or even more. More specifically, for one, two, three, four, five, six, seven, eight, nine, ten, eleven, twelve months of more or for several years.

In a further aspect, the invention relates to a method of modifying at least one target nucleic acid sequence of interest in at least one cell. Specifically, the method may comprise the steps of contacting the cell with at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. More specifically, the nucleic acid guided genome modifier chimeric or fusion protein of the disclosed method may comprise: (i) at least one CRISPR-dCas protein devoid of a nucleolytic activity or any variant or mutant thereof; and (ii) at least one nucleic acid modifier component. The chimeric or fusion protein of the disclosed methods further comprises at least one of: (iii) at least one donor attachment domain (DAD) for binding a Donor nucleic acid molecule; and/or (iv) at least one repair factor recruitment domain (RFRD). The methods disclosed herein may further use (b), at least one donor nucleic acid molecule. In some embodiments, the donor molecule comprises at least one replacement sequence for incorporation in, and/or for replacing, the target nucleic acid sequence of interest. Still further, the disclosed method may use (c), at least one target recognition element or any nucleic acid sequence encoding said target recognition element. In some embodiments, the target recognition element specifically recognizes and binds the target sequence. In yet some alternative embodiments the methods of the present disclosure may use (d), at least one nucleic acid cassette or any vector or vehicle comprising at least one of: (i) the nucleic acid sequence of (a); (ii) the nucleic acid sequence of (b); (iii) the nucleic acid sequence of (c); and (iv) the nucleic acid sequence of (a) and at least one of (b) and (c). Still further, in some embodiments, the disclosed methods may use (e), at least one system or composition comprising (a) and at least one of (b) and (c).

The methods of the invention provide targeted modification (either physical or functional as discussed above) of a target nucleic acid sequence of interest, in a target cell.

In some embodiments, the CRISPR-dCas protein suitable for the nucleic acid guided genome modifier chimeric protein, complex or conjugate used in the methods of the present disclosure may be at least one of Cas9, CasX, Cas12a1, CasF, Cas14a1, an ancestral Cas and Cas14b5. In yet some further embodiments, the disclosed has reduced or abolished PAM constraint. In such PAM abolished or reduced CRISPR-dCas protein, at least one of the PBD, any fragment of the PBD, and at least one amino acid residue adjacent to the PBD, may be deleted or replaced.

In other embodiments, the nucleic acid guided genome modifier chimeric protein, complex or conjugate suitable for the methods of the invention, the nucleic acid molecule suitable for the method of the invention, and the system suitable for the methods of the invention are any of the nucleic acid guided genome modifier chimeric or fusion protein, encoding nucleic acids, and systems as defined by the present disclosure in connection with other aspects.

In some other embodiments, the cell suitable for the method of the invention may be of at least one organism of the biological kingdom Animalia.

In some embodiments, the method of the invention may be applicable for any cell of at least one organism of the biological kingdom Animalia. In some embodiments, the method of the invention may be applicable for any cell of at least one organism of the biological kingdom Animalia. In more specific embodiments, such cell may be derived from any unicellular or multicellular invertebrate or vertebrate. More specifically, cells derived from invertebrates, are cells derived from an organism of the Phylum Porifera—Sponges, the Phylum Cnidaria—Jellyfish, hydras, sea anemones, corals, the Phylum Ctenophora—Comb jellies, the Phylum Platyhelminthes—Flatworms, the Phylum Mollusca—Molluscs, the Phylum Arthropoda—Arthropods, the Phylum Annelida—Segmented worms like earthworm and the Phylum Echinodermata—Echinoderms. Still further, in some embodiments, the methods of the invention may be applicable for a cell derived from any vertebrate organism, specifically, an organism derived from any of the vertebrates groups that include Fish, Amphibians, Reptiles, Birds and Mammals (e.g., Marsupials, Primates, Rodents and Cetaceans). In some particular embodiments, the methods of the invention may be particularly applicable for modifying a target nucleic acid sequence of interest in a cell of a mammal (specifically, at least one of a human, Cattle, rodent, domestic pig (swine, hog), sheep, horse, goat, alpaca, lama and Camels), an avian, an insect, a fish, an amphibian, a reptile, a crustacean, a crab, a lobster, a snail, a clam, an octopus, a starfish, a sea-urchin, jellyfish, and worms.

Still further, the methods of the invention may be useful for mutating, deleting, inserting or replacing a target nucleic acid sequence of interest (e.g., a coding or non-coding sequence) or any fragment thereof in a eukaryotic cell, with a replacement sequence provided by the invention, using recombination (HDR). There are several types of eukaryotic cells that may be used by the methods of the invention. By way of example, eukaryotic cells may be, but are not limited to, stem cells, e.g., hematopoietic stem cells (HSCs), embryonic stem cells, totipotent stem cells, pluripotent stem cells or induced pluripotent stem cells, multipotent progenitor cells and plant cells.

Stem cells are generally known for their three unique characteristics: (i) they have the unique ability to renew themselves continuously; (ii) they have the ability to differentiate into somatic cell types; and (iii) they have the ability to limit their own population into a small number. In mammals, there are two broad types of stem cells, namely embryonic stem cells (ESCs), and adult stem cells. Stem cells may be autologous or heterologous to the subject. In order to avoid rejection of the cells by the subject's immune system, autologous stem cells are usually preferred.

Thus, in some embodiments, the eukaryotic cells according to the invention may be embryonic stem cells, or human embryonic stem cells (hESCs), that were obtained from self-umbilical cord blood just after birth. Embryonic stem cells are pluripotent stem cells derived from the early embryo that are characterized by the ability to proliferate over prolonged periods of culture while remaining undifferentiated and maintaining a stable karyotype, with the potential to differentiate into derivatives of all three germ layers. hESCs may be also derived from the inner cell mass (ICM) of the blastocyst stage (100-200 cells) of embryos generated by in vitro fertilization. However, methods have been developed to derive hESCs from the late morula stage (30-40 cells) and, recently, from arrested embryos (16-24 cells incapable of further development) and single blastomeres isolated from 8-cell embryos.

In further embodiments, the eukaryotic cells according to the invention are totipotent stem cells. Totipotent stem cells are versatile stem cells and have the potential to give rise to any and all human cells, such as brain, liver, blood or heart cells or to an entire functional organism (e.g., the cell resulting from a fertilized egg). The first few cell divisions in embryonic development produce more totipotent cells. After four days of embryonic cell division, the cells begin to specialize into pluripotent stem cells. Embryonic stem cells may also be referred to as totipotent stem cells.

In further embodiments, the eukaryotic cells according to the invention are pluripotent stem cells. Similar to totipotent stem cells, a pluripotent stem cell refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type. However, unlike totipotent stem cells, they cannot give rise to an entire organism. On the fourth day of development, the embryo forms into two layers, an outer layer which will become the placenta, and an inner mass which will form the tissues of the developing human body. These inner cells are referred to as pluripotent cells.

In still further embodiments, the eukaryotic cells that may be applicable for therapeutic methods according to the invention, are multipotent progenitor cells. Multipotent progenitor cells have the potential to give rise to a limited number of lineages. As a non-limiting example, a multipotent progenitor stem cell may be a hematopoietic cell, which is a blood stem cell that can develop into several types of blood cells but cannot into other types of cells. Another example is the mesenchymal stem cell, which can differentiate into osteoblasts, chondrocytes, and adipocytes. Multipotent progenitor cells may be obtained by any method known to a person skilled in the art.

In yet further embodiments, the eukaryotic cells according to the invention are induced pluripotent stem cells. Induced pluripotent stem cells, commonly abbreviated as iPS cells are a type of pluripotent stem cell artificially derived from a non-pluripotent cell, typically an adult somatic cell, even a patient's own. Such cells can be induced to become pluripotent stem cells with apparently all the properties of hESCs. Induction requires only the delivery of four transcription factors found in embryos to reverse years of life as an adult cell back to an embryo-like cell. For example, iPS cells could be used for autologous transplantation in a patient with a rare disease. The mutation or mutations responsible for the patient's disease state could be corrected ex vivo in the iPS cells obtained from the patient as performed by the methods of the invention and the cells may be then implanted back into the patient (i.e., autologous transplantation).

It should be understood that any of the cells disclosed herein may be used by the methods of the invention for ex vivo therapy as disclosed above.

As will be elaborated herein below, the activity of the nucleic acid modifier or effector component in the HDR enhanced nucleic acid guided genome modifier/effector chimeric protein, complex or conjugate of the present disclosure, used in the disclosed methods referred to herein, may relate in some embodiments to any modification performed in any target nucleic acid molecule or sequence. For example, any sequence encoding a product, or alternatively any non-coding sequences. Such modification in some embodiments may result (specifically in case performed on a coding sequence, or alternatively in a regulatory non-coding sequence), in modulation of the expression, stability or activity of the encoded product. Non-limiting examples for such modification may be nucleolytic distraction, methylation, demethylation, acetylation and the like. Thus, in some specific embodiments, the nucleic acid modifier protein used in the disclosed methods may be a nuclease, and the activity referred to herein may be the nucleolytic activity of the nuclease.

Thus, the disclosed methods provide means for modulating at least one target nucleic acid sequence in a cell, thereby modulating the activity and/or viability of the genetically modified cell. “Modulation” as used herein means a perturbation of function and/or activity, stability and/or structure. In certain embodiments, modulation means an increase in gene expression. In certain embodiments, modulation means a decrease in gene expression. In yet some further embodiments, modulation may further include editing functions (specifically, deletion, insertion, mutations, substitutions or replacement) performed by the HDR enhanced nucleic acid guided genome modifier chimeric or fusion protein of the present disclosure, used in the disclosed methods, on a target nucleic acid sequence.

Thus, target nucleic acid modification performed by the modifier or effector component of the nucleic acid guided genome modifier/effector chimeric protein, complex or conjugate of the invention may include, but is not limited to: mutation, deletion, insertion, replacement, binding, digestion, nicking, methylation, acetylation, ligation, recombination, helix unwinding, chemical modification, labeling, activation, and inactivation or any combinations thereof. Target nucleic acid functional modification may lead to, but is not limited to: changes in transcriptional activation, transcriptional inactivation, alternative splicing, chromatin rearrangement, pathogen inactivation, virus inactivation, change in cellular localization, compartmentalization of nucleic acid, changes in stability, and the like, any editing activity (e.g., mutation, substitution, replacement, deletion or insertion of at least a part of the target sequence), or combinations thereof. In some further embodiments, as indicated above, the nucleic acid guided genome modifier chimeric or fusion protein of the present disclosure, systems, compositions and particularly any methods thereof, in accordance with the present disclosure may target any coding or non-coding sequences, for example regulatory sequences, such sequences may include any non-coding sequence.

According to some embodiments, the target nucleic acid sequence is a gene or any fragment thereof or any non-coding sequence involved in a genetic trait, and the modification results in changes in the transcription or translation of a genetic element, by a technical procedure that may include permanently replacing, knocking-out, temporarily or permanently enhancing, shutting-off, knocking-down, and frameshifting. In some embodiments, the genetic trait is modified by editing the genetic element sequence itself, its regulatory sequences, genes regulating the gene of interest or their regulatory sequences in a regulatory chain of events.

The present disclosure provides methods for modifying and manipulating a target sequence of interest in a target cell. The terms “target nucleic acid sequence of interest”, “gene of interest”, “a target gene of interest”, “a target gene” are used interchangeably and refer in some embodiments to a nucleic acid sequence that may comprise or comprised within a gene or any fragment or derivative thereof that is comprised by the target cell (or host cell) of the invention and is intended to be replaced, specifically, by the HDR enhanced nucleic acid guided genome modifier chimeric or fusion protein of the present disclosure. The target nucleic acid sequence or gene of interest may comprise coding or non-coding DNA regions, or any combination thereof.

In some embodiments, the nucleic acid sequence of interest may comprise coding sequences and thus may comprise exons or fragments thereof that encode any product, for example, a protein or an enzyme (or fragments thereof). In other embodiments, the target nucleic acid sequence of interest may comprise non-coding sequences, as for example start codons, 5′ un-translated regions (5′ UTR), 3′ un-translated regions (3′ UTR), or other regulatory sequences, in particular regulatory sequences that are capable of increasing or decreasing the expression of specific genes within an organism. By way of example, regulatory sequences may be selected from, but are not limited to, transcription factors, activators, repressors and promoters. In further embodiments, the target nucleic acid sequence or gene of interest may comprise a combination of coding and non-coding regions.

Still further, the term “target gene of interest” or “target nucleic acid sequence of interest” as used herein refers to a gene in a eukaryotic cell or any fragment thereof to be replaced by the replacement sequence according to the invention. The target nucleic acid sequence of interest may be either identical or otherwise different, e.g., mutated with respect to the sequence of a normal target nucleic acid sequence in a healthy individual, or with respect to a frequent allele (major allele in case of polymorphism).

In some embodiments, the target gene or nucleic acid sequence of interest may be any nucleic acid sequence or gene or fragments thereof that display aberrant expression, stability, activity or function in a mammalian subject, as compared to normal and/or healthy subject. Such target gene or any fragments thereof or any target nucleic acid sequence may be in some embodiments, associated, linked or connected, directly or indirectly with at least one pathologic condition. Thus, the target nucleic acid sequence or gene of interest in some embodiments may be a nucleic acid sequence or gene that carry at least one of: (a) at least one point mutation; (b) deletion; (c) insertion; (d) rearrangement of at least one nucleotide or more, in at least one of its coding regions or non-coding regions. In some embodiments, the target nucleic acid sequence or gene of interest may comprise a sequence that differs in at least one nucleotide, from the normal and/or healthy, and/or frequent counterpart. More specifically, a target sequence that carry a mutation in its coding sequence that may be associated with a pathologic disorder. In yet some further embodiments, the replacing sequence, that may be the corresponding gene or fragment, as containing a non-mutated form of the gene of interest or fragments thereof, replaces the mutated target sequence of interest or fragment thereof, thereby resolving the undesired effects of the mutation.

In some further embodiments, the target nucleic acid sequence of interest suitable for the method of the invention may be at least one of: at least one gene encoding at least one tumor associated antigen (TAA), at least one gene encoding a protein involved in at least one metabolic disorder, at least one gene encoding a protein involved in at least one congenital disorder, at least one gene encoding receptors for at least one viral antigen, at least one gene associated with at least one inborn error of metabolism (IEM) disorder, Immunoglobulin locus, T cell receptor (TCR) locus, safe harbor site/s (SHS), and any coding sequence or non-coding sequence involved with at least one pathologic disorder. More specifically, in some embodiments, the target nucleic acid sequence for the HDR enhanced nucleic acid guided genome modifier chimeric or fusion protein of the present disclosure, may be at least one TAA.

Tumor or cancer associated antigen (TAA), as used herein may be an antigen that is specifically expressed, over expressed or differentially expressed in tumor cells. In yet some further embodiments, TAA can stimulate tumor-specific T-cell immune responses. Exemplary tumor antigens that may be applicable in the present invention, include, but are not limited to, RAGE-1, tyrosinase, MAGE-1, MAGE-2, NY-ESO-1, Melan-A/MART-1, glycoprotein (gp) 75, gp100, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGE1b/GAGED2a, MAGE-A3, MAGE-A6, LAGE-1, CAMEL, hTRT and Eph. and TRP-1. Still further, TAA may be recognized by CD8+ T cells as well as CD4+ T cells. Non limiting examples of TAA recognized by CD8+ T cells may be CSNK1A1, GAS7, HAUS3, PLEKHM2, PPP1R3B, MATN2, CDK2, SRPX (P55L), WDR46 (T2271), AHNAK (54460F), COL18A1 (S126F), ERBB2 (H197Y), TEAD1 (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPC1 (R520Q), MLL2, ASTN1, CDK4, GNL3L, SMARCD3, MAGE-A6, MED13, PAS5A WDR46, HELZ2, AFMID, CENPL, PRDX3, FLNA, KIF16B, SON, MTFR2 (D626Y), CHTF18 (L769V), MYADM (R30W), NUP98 (A359D), KRAS (G12D), CASP8 (F67V), TUBGCP2 (P293L), RNF213 (N1702S), SKIV2L (R653H), H3F3B (A48T), AP15 (R243Q), RNF10 (E572K), PHLPP1 (G566E) and ZFYVE27 (R6H). Non limiting examples of TAA recognized by CD4+ T cells may be ERBB2IP (E805G), CIRH1A (P333L), GART (V551A), ASAP1 (P941L), RND3 (P49S), LEMD2 (P495L), TNIK (S502F), RPS12 (V104I), ZC3H18 (G269R), GPD2 (E426K), PLEC (E1179K), XPO7 (P274S), AKAP2 (Q418K) and ITGB4 (S10021). Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, SIGLEC10, hTRT and Eph. Cancer antigen and tumor antigen are used interchangeably herein. The antigens may be related to cancers that include but are not limited to any of the proliferative disorders disclosed by the present disclosure, even if indicated in connection with other aspects of the invention.

In some embodiments, the target sequence targeted by the methods of the invention is the Empty Spiracles Homeobox 1 (EMX1) gene, that encodes a member of the EMX family of transcription factors. The EMX1 gene, along with its family members, are expressed in the developing cerebrum and plays a role in specification of positional identity, the proliferation of neural stem cells, differentiation of layer-specific neuronal phenotypes and commitment to a neuronal or glial cell fate. The human EMX1 gene has a sequence as provided by NCBI Accession Number NC_000002.12, range 72910949-72936691 (NCBI gene ID 2016). In yet some further embodiments, the human EMX1 protein is denoted by NCBI Accession Number NP_004088.2.

In some embodiments, the target sequence may be a target gene or any other coding and/or non-coding sequence involved in a congenital disorder. In some particular embodiments, such gene may be a gene involved in Autosomal dominant Retinitis Pigmentosa (adRP). In some further specific embodiments, the target gene may be the rhodopsin (RHO) gene. The RHO gene (also known as long Wavelength Sensitive opsin, L opsin, LWS opsin, MGC:21585, MGC:25387, Noerg1, Opn2, Ops, opsin 2, Red Opsin, Rod Opsin, RP4), encodes a photoreceptor required for image-forming vision at low light intensity and for photoreceptor cell viability after birth. The protein encoded by this gene is found in rod cells that can sense light and initiate the phototransduction cascade in rod photoreceptors. The encoded protein binds to 11-cis retinal and is activated when light hits the retinal molecule. Defects in this gene are a cause of congenital stationary night blindness. The human RHO gene has a sequence as provided by NCBI Accession Number: NC_000003.12 (range 129528639-129535344) (Gene ID 6010). In yet some further embodiments, the human RHO protein is denoted by NCBI Accession Number NP_000530.1.

In some other embodiment, the target gene may be the Myeloperoxidase (MPO) gene. Myeloperoxidase (MPO), as used herein, is the most toxic enzyme found in the azurophilic granules of neutrophils. MPO utilizes H2O2 to generate hypochlorous acid (HClO) and other reactive moieties, which kill pathogens during infections. The MPO gene is located on the long arm segment q12-24 of chromosome 17 and the primary transcriptional product of this gene consists of 11 introns and 12 exons. Alternative splicing of the MPO mRNA gives two transcripts of 3.6 and 2.9 kB. The primary translation product is an 80 kDa precursor protein that undergoes a series of modifications including cleavage of a signal peptide, N-linked glycosylation, and limited deglycosylation, to form the catalytically inactive MPO precursor (apoproMPO). In the next step, MPO gains catalytic activity by incorporation of an iron-heme molecule into the catalytic centrum. Heme is covalently attached by two ester bonds and, unique for heme containing enzymes, a third sulfonium linkage, that uniquely orients one heme molecule into the enzyme pocket. The unique configuration of the heme moiety confers MPO with very high oxidative potential, enabling chlorination at physiological pH. Cleavage of proMPO leads to a 59 kDa α-subunit and a 13.5 kDa β-subunit that are covalently attached through the heme moiety. A disulfide bridge joins the two heavy-light protomers in mature 150 kDa MPO. MPO expression levels depend upon allelic polymorphisms in the promoter region. Neutrophils are the main source of MPO where it accounts for 5% of the dry weight of the cell, making MPO the most abundant protein in neutrophils. MPO is transcribed only in promyelocytes during neutrophil differentiation in the bone marrow.

It should be further appreciated that in the context of the present disclosure, unless specifically indicated “MPO” encompasses both the MPO gene and the MPO protein. The human MPO gene has a sequence as provided by Accession Number: NC_000017.11. In yet some further specific embodiment, such sequence may comprise the nucleic acid sequence as denoted by SEQ ID NO: 136. In yet some further embodiments, the human MPO protein is denoted by Accession Number: NP_000241. Still further in some embodiments, the MPO protein may comprise the amino acid sequence as denoted by SEQ ID NO: 137. Still further in some other specific embodiment, the invention further provides the mouse MPO encoding sequence as denoted by Accession Number NC_000077, that may in some embodiments comprise the nucleic acid sequence as denoted by SEQ ID NO: 138. In yet some further embodiments, the mouse MPO protein is denoted by Accession Number: NP_034954. Still further in some embodiments, the mouse MPO protein may comprise the amino acid sequence as denoted by SEQ ID NO: 139.

MPO plays a role in suppressing the adaptive immune response. Mechanistically, MPO released from neutrophils inhibits LPS-induced DC activation as measured by decreased IL-12 production and CD86 expression consequently, limiting T cell proliferation and proinflammatory cytokine production. In contrast, a pathogenic role for MPO in driving autoimmune inflammation was also demonstrated. More specifically, increased MPO levels and activity have been observed in many inflammatory conditions and autoimmune diseases including multiple sclerosis (MS) and rheumatoid arthritis (RA). MPO plays a role in modulation of vasculature functioning, associated with chronic vascular diseases such as atherosclerosis. In the extracellular matrix (ECM), MPO works as a nitric oxide (NO)-scavenger consuming NO that leads to impaired endothelial relaxation. MPO and its oxidative species present in the atherothrombotic tissue, promotes lipid peroxidation, conversion of LDL to a highly-uptake atherogenic form, selectively modulates Apolipoprotein A-I (apoA-I) generating dysfunctional HDL particles more susceptible to degradation and impairs the ability of apoA-I to promote cholesterol efflux. Moreover, elevated systemic levels of MPO and its oxidation products are associated with increased cardiovascular risk. As indicated above, MPO has been implicated in variety of pathologic conditions, and thereof targeting the MPO gene provides a specific therapeutic tool for treating and preventing disorders or conditions caused thereby.

In yet some further embodiments, the congenital disorder may be Pseudoachondroplasia (PSACH). In some specific embodiments, the gene targeted by the methods of the invention may be the Cartilage Oligomeric Matrix Protein (COMP) gene. The protein encoded by this gene is a noncollagenous extracellular matrix (ECM) protein. It consists of five identical glycoprotein subunits, each with EGF-like and calcium-binding (thrombospondin-like) domains. Oligomerization results from formation of a five-stranded coiled coil and disulfides. Binding to other ECM proteins such as collagen appears to depend on divalent cations. Contraction or expansion of a 5 aa aspartate repeat, and other mutations can cause pseudochondroplasia (PSACH) and multiple epiphyseal dysplasia (MED). The human COMP gene has a sequence as provided by NCBI Accession Number NC_000019.10 (18782773 . . . 18791305, complement) (Gene ID 1311). In yet some further embodiments, the human COMP protein is denoted by NCBI Accession Number NP_000086.2.

Still further in some embodiments, immune checkpoint receptor proteins or ligands (such as those targeted by checkpoint inhibitors in cancer checkpoint therapy and which can block inhibitory checkpoints, restoring immune system function) and any genes encoding such receptors or ligands may be targeted by the methods of the invention. More specifically, the methods of the invention may target immune checkpoint receptor proteins or ligands that include PD-1/PD-L1 and CTLA-4/B7-1/B7-2. In some specific embodiments, the target gene is a gene encoding the PDCD1 gene. Programmed Cell Death 1 PDCD1, also known as PD-1 and CD279, encodes a cell surface membrane protein of the immunoglobulin superfamily that has a role in regulating the immune system's response to the cells of the human body by down-regulating the immune system and promoting self-tolerance by suppressing T cell inflammatory activity, thus functioning as an immune checkpoint. The human PDCD1 gene has a sequence as provided by NCBI Accession Number: NC_000002.12 (241849881 . . . 241858908, complement) (GENE ID 5133). In yet some further embodiments, the human PDCD1 protein is denoted by NCBI Accession Number: NP_005009.2. Still further, in some embodiments, the nucleic acid guided genome modifier chimeric or fusion protein of the present disclosure may be applicable for modifying the PD1 gene. In yet some further embodiments, exon 1 of the PD1 gene is targeted by the nucleic acid guided genome modifier chimeric or fusion protein of the present disclosure and systems thereof. In more specific embodiments, the PD1 gene comprises the nucleic acid sequence as denoted by SEQ ID NO: 188. In some embodiments, the PD-1 gene disclosed herein encodes the PD-1 protein that comprises the amino acid sequence as denoted by SEQ ID NO: 8, and any variants and homologs thereof.

In yet some further embodiments, the target gene may be the CTLA4 gene that encodes CTLA4. CTLA4 or CTLA-4 (cytotoxic T-lymphocyte-associated protein 4), also known as CD152 (cluster of differentiation 152), is a protein receptor that functions as an immune checkpoint and downregulates immune responses. CTLA4 is constitutively expressed in regulatory T cells but only upregulated in conventional T cells after activation. The human CTLA4 gene has a sequence as provided by NCBI Accession Number: NC_000002.12 (203867771 . . . 203873965) (GENE ID 1493). In yet some further embodiments, the human CTLA4 protein is denoted by NCBI Accession Number: NP_001032720.1.

Still further, in some embodiments, the target nucleic acid sequence of the invention may be any gene encoding, or a sequence involved in the expression of immunological receptors, specifically, T cell receptors (TCR), B cell receptors (BCR) and antibodies. According to such embodiments, the target sequence targeted by the methods of the invention may be located at the immunoglobulin locus, specifically, any one of the Immunoglobulin heavy chain locus, Immunoglobulin κ chain locus, Immunoglobulin λ chain locus, TCRβ chain locus, TCRα chain locus, TCRγ chain and the TCRδ chain locus. In yet some further embodiments, the target sequence may be a sequence enabling insertion of a desired nucleic acid sequence. In more specific embodiments, the target sequence may be within GSHs. Genomic safe harbors (GSHs) are sites in the genome able to accommodate the integration of new genetic material in a manner that ensures that the newly inserted genetic elements: (i) function predictably and (ii) do not cause alterations of the host genome posing a risk to the host cell or organism. GSHs are thus ideal sites for transgene insertion whose use can empower functional genetics studies in basic research and therapeutic applications in human gene therapy. Non-limiting examples for SHS sites applicable in the present invention include the human AAVS1 site on chromosome 19q, and the human ROSA26, and CCR5 sites. In should be appreciated that in some embodiments of the invention, GSHs may be useful as target sequences, particularly in cases where it is desired to express an exogenous nucleic acid sequence of interest in a specific cell. Such encoding sequence may be inserted in GSH sites. Non-limiting examples for such exogenous nucleic acid sequences that can be inserted in GSHs, may be any sequence encoding a receptor or chimeric receptor, for example, any chimeric antigen receptor (CAR).

In yet some further embodiments, the target nucleic acid sequence may be a gene encoding viral receptors, for example, the Integrin Subunit Beta 3 (ITGB3) gene. The ITGB3 protein product is the integrin beta chain beta 3. Integrins are integral cell-surface proteins composed of an alpha chain and a beta chain. A given chain may combine with multiple partners resulting in different integrins. Integrin beta 3 is found along with the alpha IIb chain in platelets. The human ITGB3 gene has a sequence as provided by NCBI Accession Number: NC_000017.11 (47253827 . . . 47313743) (GENE ID 3690). In yet some further embodiments, the human ITGB3 protein is denoted by NCBI Accession Number: NP_000203.2.

In some other embodiments, the cell suitable for the methods of the invention may be of at least one organism of the biological kingdom Plantae. Cells applicable in the disclosed methods are as defined in connection with other aspects of the present disclosure.

In some other embodiments, the modification of at least one target nucleic acid sequence of interest in at least one cell as discussed herein, may be performed in at least one organism of at least one of: the biological kingdom Plantae and the biological kingdom Animalia. Thus, in some embodiments, the step of contacting the cell with the disclosed HDR enhanced nucleic acid guided genome modifier chimeric or fusion protein, may be performed by administering to the discussed organism an effective amount of the HDR enhanced nucleic acid guided genome modifier chimeric or fusion protein or any systems and/or compositions thereof.

It should be understood that the modification of a target nucleic acid sequence in a cell as disclosed herein, may be performed by the methods of the invention either in vitro/ex vivo or in vivo. “In vitro” is defined herein as an artificial environment outside the membranes of a whole or partial, differentiated or undifferentiated, living organism, organ, tissue, callus or cell. In some embodiments, the term in-vitro is not inside a viable cell.

“In vivo” is defined herein as inside a whole or partial, differentiated or undifferentiated, organism, organ, tissue, callus or cell.

It should be noted that in some embodiments, the method of the invention may be applicable for modification of at least one target nucleic acid sequence of interest in at least one cell may be performed in at least one organism of at least one of: the biological kingdom Plantae and the biological kingdom Animalia. Thus, as indicated above, the invention provides in vitro or ex vivo methods for performing a targeted modification in a gene in a cell or any parts thereof or in a tissue, or alternatively, in vivo methods for performing the desired manipulation in an organism, as disclosed by the invention.

As indicated above, the invention provides methods of manipulating a nucleic acid sequence of interest in a biological reaction or in a cell either in vitro/ex vivo, or in vivo in a target organism. The invention thus provides in some embodiments thereof non-therapeutic, and well as therapeutic methods based on manipulations and modifications of nucleic acid sequences of interest in a treated subject, and therefore relates to gene therapy. The non-therapeutic applications of such methods may encompass cosmetic, diagnostic and agricultural uses.

The term “gene therapy” as herein defined, refers to the correction, modulation or ablation of at least one target gene. This term further encompasses insertion of a gene of interest into a target locus (e.g., chimeric receptors, such as CAR, TCRs, BCRs, or antibodies), or replacement of an endogenous gene with at least one nucleic acid sequence of interest. The method of the invention is also suitable for the treatment of diseases caused by the failure of a single gene, or of multiple genes (also referred to as polygenic or chromosomal) and is applicable in cases were specific mutations resulting in a defective gene or gene are identified or not.

The method of the invention is thus suitable for the treatment of diseases caused by the failure of a single gene, or of multiple genes (also referred to as polygenic or chromosomal), provided that the specific mutations resulting in a defective gene or gene are identified. Theoretically, if the dysfunctional gene is replaced with the corresponding healthy one, or alternatively, is knocked out or modulated, a cure can be achieved.

Thus, in a further aspect, the invention provides a method of curing or treating, preventing, inhibiting, reducing, eliminating, protecting or delaying the onset of a pathologic disorder or condition in a subject in need thereof. Specifically, the method of the invention may comprise the steps of administering to the subject an effective amount of at least one of: (a) a nucleic acid guided genome modifier chimeric or fusion protein, complex or conjugate having enhanced homology-directed repair, or at least one nucleic acid sequence encoding the nucleic acid guided genome modifier chimeric or fusion protein. More specifically, the nucleic acid guided genome modifier chimeric or fusion protein of the disclosed method may comprise: (i) at least one CRISPR-dCas protein devoid of a nucleolytic activity or any variant or mutant thereof; and (ii) at least one nucleic acid modifier component. The chimeric or fusion protein of the disclosed methods further comprises at least one of: (iii) at least one donor attachment domain (DAD) for binding a Donor nucleic acid molecule; and/or (iv) at least one repair factor recruitment domain (RFRD). The methods disclosed herein may further use (b), at least one donor nucleic acid molecule. In some embodiments, the donor molecule comprises at least one replacement sequence for incorporation in, and/or for replacing, a target nucleic acid sequence of interest in the genome of the treated subject. In some embodiments, such target sequence is associated directly or indirectly with the treated disorder. The methods disclosed herein may further use (c), at least one target recognition element or any nucleic acid sequence encoding the target recognition element. In some embodiments, the target recognition element specifically recognizes and binds the target sequence in the genome of at least one cell of the treated subject. In some alternative and/or additional embodiment, the methods disclosed herein may use (d), at least one nucleic acid cassette or any vector or vehicle comprising at least one of; (i) the nucleic acid sequence of (a); (i) the nucleic acid sequence of (b); (iii) the nucleic acid sequence of (c); and (iv) the nucleic acid sequence of (a) and at least one of (b) and (c). In some alternative and/or additional embodiment, the methods disclosed herein may use (e), at least one system comprising (a) and at least one of (b) and (c). In some alternative and/or additional embodiment, the methods disclosed herein may use (f), at least one cell and/or a population of cells comprising and/or modified by, at least one of: (a), (b), (c), (d) and (e). In yet some further alternative and/or additional embodiment, the methods disclosed herein may use (g), at least one composition comprising at least one of (a), (b), (c), (d), (e) and (f).

In some embodiments, the CRISPR-dCas protein suitable for the HDR enhanced nucleic acid guided genome modifier chimeric protein, complex or conjugate used by the methods of the present disclosure may be at least one of Cas9, CasX, Cas12a1, CasF, Cas14a1, an ancestral Cas and Cas14b5. In yet some further embodiments, the CRISPR-dCas protein has reduced or abolished PAM constraint. According to some embodiments, in such PAM reduced or abolished CRISPR-dCas protein, at least one of the PBD, any fragment of the PBD, and at least one amino acid residue adjacent to the PBD, may be deleted or replaced.

In some other embodiments, the nucleic acid guided genome modifier chimeric protein, complex or conjugate suitable for the method of the invention, the nucleic acid molecule suitable for the method of the invention, the system suitable for the method of the invention, the host cell suitable for the method of the invention and the composition suitable for the method of the invention may be as defined in connection with other aspects of the present disclosure.

In some embodiments, the subject of the method of the invention may be of the biological kingdom Animalia or of the biological kingdom Plantae.

As mentioned above, the invention concerns any eukaryotic organism and as such may be also applicable for members of the biological kingdom Plantae.

In more specific embodiments, the HDR enhanced nucleic acid guided genome modifier/effector chimeric protein, complex or conjugate of the invention and any systems, compositions and methods thereof, may be applicable for any plant. In more specific embodiments, such plant may be a dioecious plant or monoecious plant.

More specifically, in some embodiments the organism of the biological kingdom Plantae may be a dioecious plant, specifically, a plant presenting biparental reproduction. In some specific embodiments, the plant manipulated by the methods and systems of the invention may be of the family Cannabaceae, specifically, any one of Cannabis (hemp, marijuana) and Humulus (hops). In more specific embodiments, the plant of the family Cannabaceae may be Cannabis (hemp, marijuana). In yet some further embodiments, the plant of the family Cannabaceae may be Humulus (hops).

In some embodiments, any plants are applicable in the present invention, for example, any model plants such as, Arabidopsis, Tobacco, Solanum licopersicum, Solanum tuberosum.

In yet some further embodiments, Canola, Cereals (Corn wheat, Barley), rice, sugarcane, Beet, Cotton, Banana, Cassava, sweet potato, lentils, chickpea, peas, Soy, nuts, peanuts, Lemna, Apple, may be applicable in the present invention.

A non-comprehensive list of useful annual and perennial, domesticated or wild, monocotyledonous or dicotyledonous land plant or Algae—(i.e. unicellular or multicellular algae including diatoms, microalgae, ulva, nori, gracilaria), applicable in accordance with the invention may include but are not limited to crops, ornamentals, herbs (i.e., labiacea such as sage, basil and mint, or lemon grass, chives), grasses (i.e., lawn and biofuel grasses and animal feed grasses), cereals (i.e., rice, wheat, rye, oats, corn), legumes (i.e. soy, beans, lentils, chick peas, peas, peanuts), leafy vegetables (i.e. kale, bok-choi, cress, lettuce, spinach, cabbage), Amaranthacea (i.e. sugar beet, beet, quinoa, spinach), Compositea (i.e. sunflower, lettuce, aster), Malvaceae (i.e. cotton, cacao, okra, hibiscus), cucurbits (i.e., cucumber, squash, melon, watermelon), Solanaceous species (i.e. tobacco, potato, tomato, petunia and pepper), Umbellifera (i.e. carrot, celery, dill, parsley, cumin), Crucifera (i.e., oilseed rape, mustard, brassicas, cauliflower, radish), Sesame, the monocot Aspargales (i.e. onion, garlic, leek, asparagus, vanilla, lilies, tulips, narcissus), Myrtacea (i.e., Eucalyptus, pomegranate, guava), Subtropical fruit trees (i.e. Avocado, Mango, Litchi, papaya), Citrus (i.e. orange, lemon, grapefruit), Rosacea (i.e. apple, cherry, plum, almond, roses), berry-plants (i.e. grapes, mulberries, blueberries, raspberry, strawberry), nut trees (i.e. macademia, hazelnut, pecan, walnut, chestnuts, brazil nut, cashew), banana and plantain, palms (i.e., oil-palm, coconut and dates), evergreen, coniferous or deciduous trees, woody species.

Still further, plants useful for food, beverage (i.e. passion fruit, citrus, Paulinia, Humulus), biofuel (i.e. Ricinus, maize, soy, oil-palm, Jatropha, Switchgrass) biopesticide (i.e. pyrethrum, neem tree), ornament (i.e. cut, gardened or potted flower species such as lilies, roses, carnations, Poinsettia, petunia, cactuses, daffodils, shrubs, climbing plants, junipers), fibers (i.e. cotton, flax, agave, cannabis), construction, paper and cardboard, pigments, latex (i.e. Hevea), alcohol (i.e. grape, rye, sugarcane, cereals, fruit), oil (i.e. soy, peanut, sesame, maize, canola, rape, olive, oil-palm, argan, nuts), sugar (i.e. maize, sugarcane, sugar-beet, maple), fruit and vegetable, tea, coffee, cacao, olives, spices (i.e. ginger, cinnamon, curry, fenugreek, cumin, pepper, cardamom), chemical extraction, phytochemicals, antioxidants (i.e. plants producing phenolics, carotenoids, anthocyanins, and tocopherols), non-sugar sweeteners (i.e. Stevia), medicinal or bioactive compound producing plants (i.e. poppy, alkaloid producing species, cannabis, willow, foxglove, Cinchona (quinine) and Artemisia (antimalarial)), lawns, research model plants (i.e. Arabidopsis, tobacco), cosmetically useful plants (i.e. argan, aloe, jojoba, lavender, chamomile, tea-tree, geranium), industrially useful plants, industrial feedstock plants, animal (incl. mammal, fish and insect) feed and fodder plants, bio-amelioration plants, fertilization, breeding stock, as encompassed by the invention.

In yet some further embodiments, nonfood products made from plants include essential oils, natural dyes, pigments, waxes, resins, tannins, alkaloids, amber and cork. Products derived from plants include soaps, shampoos, perfumes, cosmetics, paint, varnish, turpentine, rubber, latex, lubricants, linoleum, plastics, inks, and gums.

In some embodiments, the methods and systems of the invention may be applicable for any plant parts, specifically, leaves, shoots, seedlings, fronds, cane, seeds, fruit, nuts, berries, flowers, trunks, branches, bark, roots, corms, rhizomes bulbs and stems, latexes and exudates.

Other plants that are pests (i.e., Orobanchaceae, Cuscuta) or weeds (broad-leaf weeds such as Convolvulus, Datura and monocot grasses such as crab grass, Cyperus).

In some alternative embodiments, the subject of the method of the invention may be of the biological kingdom Animalia is a mammalian subject.

Thus, the methods of the invention may be applicable for any subject of the biological kingdom Animalia. It should be understood that an organism of the Animalia kingdom in accordance with the invention includes any invertebrate or vertebrate organism.

More specifically, Invertebrates are animals that neither possess nor develop a vertebral column (commonly known as a backbone or spine), derived from the notochord. This includes all animals apart from the subphylum Vertebrata. More specifically, invertebrates include the Phylum Porifera—Sponges, the Phylum Cnidaria—Jellyfish, hydras, sea anemones, corals, the Phylum Ctenophora—Comb jellies, the Phylum Platyhelminthes—Flatworms, the Phylum Mollusca—Molluscs, the Phylum Arthropoda-Arthropods, the Phylum Annelida—Segmented worms like earthworm and the Phylum Echinodermata—Echinoderms. Familiar examples of invertebrates include insects; crabs, lobsters and their kin; snails, clams, octopuses and their kin; starfish, sea-urchins and their kin; jellyfish and worms.

In some embodiments the invention may be applicable for any organism of the phylum arthropod that are invertebrate animals having an exoskeleton (external skeleton), a segmented body, and paired jointed appendages. Arthropods form the phylum Euarthropoda, which includes insects, arachnids, myriapods, and crustaceans. Insects or Insecta are hexapod invertebrates and the largest group within the arthropod phylum. Definitions and circumscriptions vary; usually, insects comprise a class within the Arthropoda. As used here, the term Insecta is synonymous with Ectognatha. Insects have a chitinous exoskeleton, a three-part body (head, thorax and abdomen), three pairs of jointed legs, compound eyes and one pair of antennae. Insects are the most diverse group of animals; they include more than a million described species and represent more than half of all known living organisms. Insects can be divided into two groups historically treated as subclasses: wingless insects, known as Apterygota, and winged insects, known as Pterygota. The Apterygota consist of the primitively wingless order of the silverfish (Zygentoma). Archaeognatha make up the Monocondylia based on the shape of their mandibles, while Zygentoma and Pterygota are grouped together as Dicondylia. The Zygentoma themselves possibly are not monophyletic, with the family Lepidotrichidae being a sister group to the Dicondylia (Pterygota and the remaining Zygentoma). Paleoptera and Neoptera are the winged orders of insects differentiated by the presence of hardened body parts called sclerites, and in the Neoptera, muscles that allow their wings to fold flatly over the abdomen. Neoptera can further be divided into incomplete metamorphosis-based (Polyneoptera and Paraneoptera) and complete metamorphosis-based groups. It should be noted that the present invention is applicable for any of the insects of any of the groups and species disclosed herein.

Still further, many insects are considered pests by humans. Insects commonly regarded as pests include those that are parasitic (e.g., lice, bed bugs), transmit diseases (mosquitoes, flies), damage structures (termites), or destroy agricultural goods (locusts, weevils). Insects considered pests of some sort occur among all major living orders with the exception of Ephemeroptera (mayflies), Odonata, Plecoptera (stoneflies), Embioptera (webspinners), Trichoptera (caddisflies), Neuroptera (in the broad sense), and Mecoptera (also, the tiny groups Zoraptera, Grylloblattodea, and Mantophasmatodea). Of particular interest of this group is the Mosquito. More specifically, in some embodiments, the invention may be suitable for insects such as mosquito for example. Mosquitoes are a group of about 3500 species of small insects that are a type of fly (order Diptera). Within that order they constitute the family Culicidae. Superficially, mosquitoes resemble crane flies (family Tipulidae) and chironomid flies (family Chironomidae). It should be appreciated that in some embodiments, the term mosquito, as used herein includes all genera encompassed by the subfamilies Anophelinae and Culicinae. In yet some further embodiments, mosquito as used herein include, but is not limited to any mosquito of the following genera, Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina, and Wyeomyia. Females of most species are ectoparasites, whose tube-like mouthparts (called a proboscis) pierce the hosts' skin to consume blood. Though the loss of blood is seldom of any importance to the victim, the saliva of the mosquito often causes an irritating rash that is a serious nuisance. Much more serious though, are the roles of many species of mosquitoes as vectors of diseases. In passing from host to host, some transmit extremely harmful infections such as malaria, yellow fever, Chikungunya, West Nile virus, dengue fever, filariasis, Zika virus and other arboviruses, rendering it the deadliest animal family in the world.

In some embodiments of the present invention is the bee. Bees are flying insects closely related to wasps and ants, known for their role in pollination and, in the case of the best-known bee species, the western honeybee, for producing honey and beeswax. Bees are a monophyletic lineage within the superfamily Apoidea and are presently considered a clade, called Anthophila. There are nearly 20,000 known species of bees in seven recognized biological families, specifically, Andrenidae, Apidae, Colletidae, Halictidae, Megachilidae, Melittidae, Stenotritidae. Some species including honeybees, bumblebees, and stingless bees live socially in colonies. It should be understood that the present invention encompasses any of the bee species of any of the bee families indicated herein. Still further, the invention may be useful for crustacean organisms. Crustaceans, as used herein, form a large, diverse arthropod taxon which includes crabs, lobsters, crayfish, shrimp, krill, woodlice, and barnacles, which are all encompassed by the present invention. The crustacean group is usually considered as a paraphyletic group and comprises all animals in the Pancrustacea clade other than hexapods. Some crustaceans are more closely related to insects and other hexapods than they are to certain other crustaceans. In some embodiments, such crustaceans may be shrimp. The term shrimp is used to refer to decapod crustaceans and covers any of the groups with elongated bodies and a primarily swimming mode of locomotion i.e., Caridea and Dendrobranchiata.

In yet some further embodiments, the invention may be useful for organisms of the subphylum Chelicerata that is one of the major subdivisions of the phylum Arthropoda and includes the sea spiders, arachnids, and several extinct lineages. In more specific embodiments, the invention may be useful for organisms of the Arachnida that are a class including spiders (the largest order in the class), scorpions, Acari (ticks, mites), harvestmen, and solifuges.

Still further, in some embodiments, the methods of the invention may be applicable for a vertebrate organism. Vertebrates comprise all species of animals within the subphylum Vertebrata (chordates with backbones). The animals of the vertebrates group include Fish, Amphibians, Reptiles, Birds and Mammals (e.g., Marsupials, Primates, Rodents and Cetaceans).

Vertebrates represent the overwhelming majority of the phylum Chordata, with currently about 66,000 species described. Vertebrates include the jawless fish and the jawed vertebrates, which include the cartilaginous fish (sharks, rays, and ratfish) and the bony fish.

Still further, in some embodiments, the subject of the invention may be any one of a human or non-human mammal, an avian, an insect, a fish, an amphibian, a reptile, a crustacean, a crab, a lobster, a snail, a clam, an octopus, a starfish, a sea-urchin, jellyfish, and worms.

In more specific embodiments, the subject of the invention may be a mammal. In yet some further embodiments, such mammalian organisms may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks).

In yet some further embodiments, the invention may be applicable for any organism of the order primates. More specifically, primates are divided into two distinct suborders, the first is the strepsirrhines that includes lemurs, galagos, and lorisids. The second is haplorhines—that includes tarsier, monkey, and ape clades, the last of these including humans. In yet some further embodiments, the invention may be applicable for any organism of the subfamily Homininae, that includes the hylobatidae (gibbons) and the hominidae that includes ponqunae (orangutans) and homininae [gorillini (gorilla) and hominini ((panina(chimpanzees) and hominina (humans))].

In some specific embodiment, the methods of the invention may be applicable for a mammal that may be at least one of a Cattle, domestic pig (swine, hog), sheep, horse, goat, alpaca, lama and Camels.

In some embodiments, the invention may be applicable for subject of the Order Artiodactyla, including members of the family Suidae, subfamily Suinae and Genus Sus, and members of the family Bovidae, subfamily Bovinae including ungulates. More specifically, domestic cattle, bison, African buffalo, the water buffalo, the yak. Of particular interest in the present invention are domestic cattle being the most widespread species of the genus Bos and are most commonly classified collectively as Bos taurus. More specifically, the subject the invention as well as the methods disclosed herein above offer great economic advantage for any industrial or agricultural use of animals, specifically, livestock. Thus, in some specific embodiments, the invention may be applicable for mammalian livestock, specifically those used for meat, milk and leather industries. Livestock are domesticated animals raised in an agricultural setting to produce labor and commodities such as meat, eggs, milk, fur, leather, and wool. The term includes but is not limited to Cattle, sheep, domestic pig (swine, hog), horse, goat, alpaca, lama and Camels. Of particular interest are cattle applicable in the meat and milk industry, as well as in the leather industry. More specifically, in certain embodiments, the subject of the invention may be Cattle, colloquially cows, that are the most common type of large, domesticated ungulates, that belong to the Bovidae family.

Still further, the Bovidae are the biological family of cloven-hoofed, ruminant mammals that includes bison, African buffalo, water buffalo, antelopes, wildebeest, impala, gazelles, sheep, goats, muskoxen. The biological subfamily Bovinae includes a diverse group of ten genera of medium to large-sized ungulates, including domestic cattle, bison, African buffalo, the water buffalo, the yak, and the four-horned and spiral-horned antelopes. Of particular interest in the present invention may be the domestic cattle are the most widespread species of the genus Bos and are most commonly classified collectively as Bos taurus. More specifically, Bos is the genus of wild and domestic cattle. Bos can be divided into four subgenera: Bos, Bibos, Novibos, and Poephagus. Subgenus Bos includes Bos primigenius (cattle, including aurochs), Bos primigenius primigenius (aurochs), Bos primigenius taurus (taurine cattle, domesticated) and Bos primigenius indicus (zebu, domesticated).

In yet some further embodiments, rodents may be of particular relevance since it represents the most popular and commonly accepted animal model in research.

Thus, in some further embodiment, the methods of the invention may be applicable for a mammal such as a rodent. Rodents are mammals of the order Rodentia, which are characterized by a single pair of continuously growing incisors in each of the upper and lower jaws. Rodents are the largest group of mammals. Non-limiting examples for such rodents that are applicable in the present invention, appear in the following list of rodents, arranged alphabetically by suborder and family. Suborder Anomaluromorpha includes the anomalure family (Anomaluridae) [anomalure (genera Anomalurus, Idiurus, and Zenkerella)], the spring hare family (Pedetidae) [spring hare (Pedetes capensis)]. The suborder Castorimorpha includes the beaver family (Castoridae) [beaver (genus Castor), giant beaver (genus Castoroides; extinct)], the kangaroo mice and rats (family Heteromyidae) [kangaroo mouse (genus Microdipodops), kangaroo rat (genus Dipodomys), pocket mouse (several genera)], the pocket gopher family (Geomyidae) [pocket gopher (multiple genera)]. Suborder Hystricomorpha, includes the agouti family (Dasyproctidae), acouchy (genus Myoprocta) [agouti (genus Dasyprocta)], the American spiny rat family (Echimyidae), the American spiny rat (multiple genera), the blesmol family (Bathyergidae) [blesmol (multiple genera)], the cane rat family (Thryonomyidae) [cane rat (genus Thryonomys)], the cavy family (Caviidae) [capybara (Hydrochoerus hydrochaeris), guinea pig (Cavia porcellus) mara (genus Dolichotis)], the chinchilla family (Chinchillidae) [chinchilla (genus Chinchilla), viscacha (genera Lagidium and Lagostomus)], the chinchilla rat family (Abrocomidae) [chinchilla rat (genera Cuscomys and Abrocoma)], the dassie rat family (Petromuridae) [dassie rat (Petromus typicus)], the degu family (Octodontidae) [degu (genus Octodon)], the diatomyid family (Diatomyidae), the giant hutia family (Heptaxodontidae), the gundi family (Ctenodactylidae) [gundi (multiple genera)], the hutia family (Capromyidae) [hutia (multiple genera)], the New World porcupine family (Erethizontidae) [New World porcupine (multiple genera)], the nutria family (Myocastoridae) [nutria (Myocastor coypus)], the Old World porcupine family (Hystricidae) [Old World porcupine (genera Atherurus, Hystrix, and Trichys)], the paca family (Cuniculidae) [paca (genus Cuniculus)], the pacarana family (Dinomyidae) [pacarana (Dinomys branickii)], the tuco-tuco family (Ctenomyidae) [tuco-tuco (genus Ctenomys)]. The suborder Myomorpha that includes the cricetid family (Cricetidae) [American harvest mouse (genus Reithrodontomys), cotton rat (genus Sigmodon), deer mouse (genus Peromyscus), grasshopper mouse (genus Onychomys), hamster (various genera), golden hamster (Mesocricetus auratus), lemming (various genera) maned rat (Lophiomys imhausi), muskrat (genera Neofiber and Ondatra), rice rat (genus Oryzomys), vole (various genera), meadow vole (genus Microtus), woodland vole (Microtus pinetorum), water rat (various genera), woodrat (genus Neotoma), dipodid family (Dipodidae), birch mouse (genus Sicista), jerboa (various genera), jumping mouse (genera Eozapus, Napaeozapus, and Zapus)], the mouselike hamster family (Calomyscidae), the murid family (Muridae) [African spiny mouse (genus Acomys), bandicoot rat (genera Bandicota and Nesokia), cloud rat (genera Phloeomys and Crateromys), gerbil (multiple genera), sand rat (genus Psammomys), mouse (genus Mus), house mouse (Mus musculus), Old World harvest mouse (genus Micromys), Old World rat (genus Rattus), shrew rat (various genera), water rat (genera Hydromys, Crossomys, and Colomys), wood mouse (genus Apodemus)], thenesomyid family (Nesomyidae), African pouched rat (genera Beamys, Cricetomys, and Saccostomus)], the Oriental dormouse family (Platacanthomyidae)[Asian tree mouse (genera Platacanthomys and Typhlomys)], the spalacid family (Spalacidae) [bamboo rat (genera Rhizomys and Cannomys), blind mole rat (genera Nannospalax and Spalax), zokor (genus Myospalax), suborder Sciuromorpha], the dormouse family (Gliridae) [dormouse (various genera), desert dormouse (Selevinia betpakdalaensis)], the mountain beaver family (Aplodontiidae) [mountain beaver (Aplodontia rufa)], the squirrel family (Sciuridae) [chipmunk (genus Tamias), flying squirrel (multiple genera), ground squirrel (multiple genera), suslik (genus Spermophilus), marmot (genus Marmota), groundhog (Marmota monax), prairie dog (genus Cynomys), tree squirrel (multiple genera)]. In yet some further embodiments, the subject of the invention may be a mouse. A mouse, plural mice, is a small rodent characteristically having a pointed snout, small, rounded ears, a body-length scaly tail and a high breeding rate. The best-known mouse species is the common house mouse (Mus musculus). Species of mice are mostly found in Rodentia and are present throughout the order. Typical mice are found in the genus Mus.

In yet some further embodiments, the organism applicable in the methods of the invention, may be avian organisms. In yet some further specific embodiments, the invention may be suitable for birds. More specifically, domesticated and undomesticated birds are also suitable organisms for the invention.

Therefore, in certain embodiments, the avian organism of the invention may be any one of a domesticated and an undomesticated bird. In more specific embodiment, the avian organism may be any one of a poultry or a game bird. In some specific embodiments, the avian organism may be of the order Galliformes which comprise without limitation, chicken, quail, turkey, duck, Gallinacea sp, goose, pheasant and other fowl. The term “avian” relates to any species derived from birds characterized by feathers, toothless beaked jaws, the laying of hard-shelled eggs, a high metabolic rate, a four-chambered heart, and a lightweight but strong skeleton. The term “hen” includes all females of the avian species.

In yet some further embodiments, the methods of the invention may be applicable for treating mammalian subjects, specifically, human subjects.

As discussed herein, the present disclosure provides therapeutic methods.

In some other embodiments, the disclosed methods may be applicable to any pathologic disorder, for example, any one of a proliferative disorder, a metabolic disorder, a congenital disorder, an immune-related condition, an inflammatory condition, a disorder caused by a pathogen, an autoimmune disorder and an IEM disorder.

In some embodiments, the disclosed therapeutic methods are applicable for the treatment and prophylaxis of proliferative disorders.

Proliferative disorders, such as cancer, may also be classified as genetic disorders or conditions, as they may result from a defect in a single or multiple genes. Some non-limiting examples of cancers that are classified as genetic disorders or conditions are FAP (familial adenomatous polyposis) or HNPCC (hereditary non-polyposis colon cancer) and breast or ovarian cancers that are associated with inherited mutations in either of the tumor suppressor BRCA1 or BRCA2 genes. The latter examples may be classified as polygenic (or chromosomal) genetic disorders. Approximately five to ten percent of cancers are entirely hereditary. Thus, proliferative disorders may also be treated by the methods of the invention.

More specifically, as used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for treatment of a patient suffering from any one of non-solid and solid tumors. Malignancy, as contemplated in the present invention may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes. Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. In yet some further embodiments, the methods of the invention may be applicable for any of the proliferative disorders discussed herein. In more specific and non-limiting embodiments, the methods of the invention may be specifically applicable for at least one of non-small cell lung cancer (NSCLC) melanoma, renal cell cancer, ovarian carcinoma and breast carcinoma. Still further, it should be appreciated that the methods disclosed herein are applicable for any neoplastic disorder, specifically, any malignant or non-malignant proliferative disorder. In yet some further embodiments, the method and uses of the present disclosure are applicable for any cancer. Thus, in some illustrative and non-limiting embodiments, the methods and uses of the present disclosure may be applicable for any one of: Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

Still further, in some embodiments, where the disorder treatable by the therapeutic methods of the present invention is a proliferative disorder, immune-check point proteins may be targeted by the HDR enhancing nucleic acid guided genome modifier chimeric or fusion protein and systems used by the disclosed methods. In this connection, any of the immune-checkpoint proteins disclosed by the present disclosure are also applicable in the present aspect.

In yet some further embodiments, the therapeutic methods provided by the present disclosure may be applicable for the treatment of metabolic disorders, for example, diabetes. Diabetes mellitus is a syndrome characterized by disordered metabolism and inappropriately high blood sugar (hyperglycaemia) resulting from either low levels of the hormone insulin or from abnormal resistance to insulin's effects coupled with inadequate levels of insulin secretion to compensate. The characteristic symptoms are excessive urine production (polyuria), excessive thirst and increased fluid intake (polydipsia), and blurred vision, these symptoms are likely absent if the blood sugar is only mildly elevated.

There are three main forms of diabetes: type I, type II and gestational diabetes (occurs during pregnancy). Type I diabetes mellitus is characterized by loss of the insulin-producing beta cells of the islets of Langerhans in the pancreas, leading to a deficiency of insulin. The main cause of this beta cell loss is a T-cell mediated autoimmune attack. There is no known preventative measure that can be taken against type I diabetes. Most affected people are otherwise healthy and of a healthy weight when onset occurs. Sensitivity and responsiveness to insulin are usually normal, especially in the early stages. Type I diabetes can affect children or adults and was traditionally termed “juvenile diabetes” as it represents a majority of cases of diabetes affecting children.

The principal treatment of type I diabetes, even from the earliest stages, is replacement of insulin combined with careful monitoring of blood glucose levels using blood testing monitors. Without insulin, diabetic ketoacidosis can develop and may result in coma or death. Emphasis is also placed on lifestyle adjustments (diet and exercise) though these cannot reverse the loss. Apart from the common subcutaneous injections, it is also possible to deliver insulin by a pump, which allows continuous infusion of insulin 24 hours a day at present levels, and the ability to program doses (a bolus) of insulin as needed at mealtimes.

In some more specific embodiments, different strategies or targets may be employed in order to treat diabetes according to the method of the invention. In some embodiments, the targeting element may relate to T cell receptor (TCR). In another embodiments, the targeting element may relate to the Renalase, FAD Dependent Amine Oxidase (RNLS) gene or Inositol-requiring transmembrane kinase endoribonuclease-1α (IRE1α) IRE1alpha gene or to the RE1 Silencing Transcription Factor (REST) gene.

Still further, in some specific embodiments, the methods of the invention may be applicable for treating and curing congenital disorders. A congenital disorder is any one of monogenic or chromosomal or multifactorial.

As indicated above, the invention provides methods for curing genetic disorders. Specifically, by replacing, mutating, deleting or inserting a sequence into a mal functioning or mutated gene or fragment/s thereof that are associated with the genetic condition using the methods of the invention. A genetic disorder or condition as herein defined is a disease caused by an abnormality in the DNA sequence of an individual. Abnormalities as used herein refer to a small mutation in a single gene. A genetic disorder or condition may be a heritable disorder and as such may be present from before birth. Other genetic disorders or conditions are caused by misregulation of a gene or new mutations or changes to the DNA.

Based on their genetic contribution, human genetic disorders or conditions can be classified as monogenic (i.e., which involve mutations in a single gene), chromosomal (also referred to as polygenic), or multifactorial genetic diseases. Monogenic diseases are caused by alterations in a single gene.

A hereditary disease may result unexpectedly when two healthy carriers of a defective recessive gene reproduce but can also happen when the defective gene is dominant. The term “mutation” as herein defined refers to a change in the nucleotide sequence of the genome of an organism. Mutations result from unrepaired damage to DNA or to RNA genomes (typically caused by radiation or chemical mutagens), from errors in the process of replication, or from the insertion or deletion of segments of DNA by mobile genetic elements. Mutations may or may not produce observable (phenotypic) changes in the characteristics of an organism. Mutation can result in several different types of change in the DNA sequence; these changes may have no effect, alter the product of a gene, or prevent the gene from functioning properly or completely. There are generally three types of mutations, namely single base substitutions, insertions and deletions and mutations defined as “chromosomal mutations”.

The term “single base substitutions” as herein defined refers to a single nucleotide base which is replaced by another. These single base changes are also called point mutations. There are two types of base substitutions, namely, “transition” and “transversion”. When a purine base (i.e., Adenosine or Thymine) replaces a purine base or a pyrimidine base (Cytosine, Guanine) replaces a pyrimidine base, the base substitution mutation is termed a “transition”. When a purine base replaces a pyrimidine base or vice-versa, the base substitution is called a “transversion”.

Single base substitutions may be further classified according to their effect on the genome, as follows:

In missense mutations the new base alters a codon, resulting in a different amino acid being incorporated into the protein chain. As a non-limiting example, the disease sickle cell anemia is a result of a single base substitution that is a missense mutation. In sickle cell anemia, the 17th nucleotide of the gene for the beta chain of haemoglobin (haem) is mutated from an ‘a’ to a ‘t’. This changes the codon from ‘gag’ to ‘gtg’, resulting in the 6th amino acid of the chain being changed from glutamic acid to Valine. This alteration to the beta globin gene alters the quaternary structure of haemoglobin, which has a profound influence on the physiology and wellbeing of the individual.

In nonsense mutations the new base changes a codon that specified an amino acid into one of the stop codons (taa, tag, tga). This will cause translation of the mRNA to stop prematurely and a truncated protein to be produced. This truncated protein will be unlikely to function correctly. Nonsense mutations are the molecular basis for between 15% to 30% of all inherited diseases. Some non-limiting examples include Cystic fibrosis, haemophilia, retinitis pigmentosa and Duchenne muscular dystrophy.

In silent mutations no change in the final protein product occurs and thus the mutation can only be detected by sequencing the gene. Most amino acids that make up a protein are encoded by several different codons (see genetic code). So, if for example, the third base in the ‘cag’ codon is changed to an ‘a’ to give ‘caa’, a glutamine (Q) would still be incorporated into the protein product, because the mutated codon still codes for the same amino acid. These types of mutations are ‘silent’ and have no detrimental effect. Mutation may also arise from insertions of nucleic acids into the DNA or from duplication or deletions of nucleic acids therefrom. As herein defined, the term “insertions and deletions” refers to extra base pairs that are added or deleted from the DNA of a gene, respectively. The number of bases can range from a few to thousands. Insertions and deletions of one or two bases or multiples of one or two bases cause, inter alia, frame shift mutations (i.e., these mutations shift the reading frame of the gene). These can have devastating effects because the mRNA is translated in new groups of three nucleotides and the protein being produced may be useless.

Insertions and deletions of three or multiples of three bases may be less serious because they preserve the open reading frame. However, a number of trinucleotide-repeat diseases exist including, for example, Huntington's disease and fragile X syndrome.

In Huntington's disease, for example, the repeated trinucleotide is ‘cag’. This adds a string of glutamines to the Huntington protein. The abnormal protein produced interferes with synaptic transmission in parts of the brain leading to involuntary movements and loss of motor control.

Genetic disorders (or conditions, diseases) that may be cured by the methods of the invention may be further classified as “recessive” and “dominant” as well as autosomal and X-linked (relating to the chromosome the gene is on).

The term “Autosomal dominant disorder” as referred to herein encompasses genetic disorders or diseases, in which only one mutated copy of the gene is required for a person to be affected. Each affected person usually has one affected parent. Some non-limiting examples of autosomal dominant genetic diseases are Huntington's disease, Neurofibromatosis 1, and Marfan syndrome.

The term “autosomal recessive disorder” as referred to herein, encompasses genetic diseases, in which two copies of the gene should be mutated for a person to be affected. An affected person usually has unaffected parents who each carry a single copy of the mutated gene (and are referred to as carriers). Some non-limiting examples of autosomal recessive disorders include Cystic fibrosis, sickle cell anemia, Tay-Sachs disease, spinal muscular atrophy, Sickle-cell disease (SCD) and phenylketonuria (PKU) which is an autosomal recessive metabolic genetic disorder.

The term “X-linked dominant” as herein defined refers to disorders that are caused by mutations in genes on the X chromosome. Males are more frequently affected than females, and the chance of passing on an X-linked dominant disorder differs between men and women. Some X-linked dominant conditions include, but are not limited to Aicardi Syndrome, and Hypophosphatemia. X-linked disorders may also be classified as “recessive X-linked”. Recessive X-linked disorders as herein defined are also caused by mutations in genes on the X chromosome. Males are more frequently affected than females, and the chance of passing on the disorder differs between men and women. Some non-limiting examples of recessive X-linked disorders are Hemophilia A, Duchenne muscular dystrophy, Color blindness, Muscular dystrophy, Androgenetic alopecia and G-6-PD (Glucose-6-phosphate dehydrogenase) deficiency.

Genetic disorders may also be Y-linked. The term “Y-linked disorders” as herein defined refers to genetic diseases that are caused by mutations on the Y chromosome. Only males can get them, and all of the sons of an affected father are affected.

Genetic disorders may also be classified as “Mitochondrial”. The term “Mitochondrial diseases” as herein defined refers to maternal inheritance, and only applies to genes in mitochondrial DNA. Because only egg cells contribute mitochondria to the developing embryo, only females can pass on mitochondrial conditions to their children. A non-limiting example of a mitochondrial genetic disease is Leber's Hereditary Optic Neuropathy (LHON).

In further embodiments, the genetic disorder may be a multifactorial genetic disease. Examples of multifactorial genetic diseases include but are not limited to breast and ovarian cancers that are associated with the BRCA1 or BRCA2 gene, Alzheimer's disease, some forms of colon cancer, e.g., familial adenomatous polyposis (FAP) or hereditary non-polyposis colon cancer (HNPCC) as well as hypothyroidism.

Currently around 4,000 genetic disorders or conditions are known, with more being discovered. Most disorders or conditions are quite rare and affect one person in every several thousands or millions. Interestingly, Cystic fibrosis is one of the most common genetic disorders; around 5% of the population of the United States carry at least one copy of the defective gene.

The method of the invention may also be used for the treatment of orphan diseases. The term “orphan disease” as herein defined refers to a rare disease, which affects a small percentage of the population. Most rare diseases are genetic, and thus are present throughout the person's entire life, even if symptoms do not immediately appear. Many rare diseases appear early in life, and about 30 percent of children with rare diseases will die before reaching their fifth birthday. A disease may be considered rare in one part of the world, or in a particular group of people, but still be common in another. A rare disease was defined in the Orphan Drug Act of 1983 as one that afflicts fewer than 200,000 people in a nation. According to the National Institute of Health, some non-limiting examples of orphan diseases are Cystic fibrosis, Ataxia telangiectasia and Tay-Sachs, to name but few.

In some embodiments, the genetic disorder or condition encompassed by the invention is a monogenic genetic disease, which may be, but is not limited to Duchenne muscular dystrophy, Cystic Fibrosis, Tay-Sachs disease (also known as GM2 gangliosidosis or hexosaminidase A deficiency), Ataxia-Telangiectasia (A-T), Sickle-cell disease (SCD), or sickle-cell anemia (SCA or anemia), Lesch-Nyhan syndrome (LNS, also known as Nyhan's syndrome, Amyotrophic Lateral Sclerosis, Cystinosis, Kelley-Seegmiller syndrome and Juvenile gout), color blindness, Haemochromatosis (or haemosiderosis), Haemophilia, Phenylketonuria (PKU), Phenylalanine Hydroxylase Deficiency disease, Polycystic kidney disease (PKD or PCKD, also known as polycystic kidney syndrome), Alpha-galactosidase A deficiency, Fabry disease, Anderson-Fabry disease, Angiokeratoma Corporis Diffusum, CADASIL (cerebral autosomal dominant arteriopathy with subcortical infarcts and leukoencephalopathy), Cerebral arteriopathy with subcortical infarcts and leukoencephalopathy, Cerebral autosomal dominant ateriopathy with subcortical infarcts and leukoencephalopathy, Carboxylase Deficiency, Multiple (Late-Onset), Cerebroside Lipidosis syndrome, Gaucher's disease, Choreoathetosis self-mutilation hyperuricemia syndrome, Classic Galactosemia, Galactosemia, Crohn's disease, also known as Crohn syndrome and regional enteritis, Incontinentia Pigmenti (also known as “Bloch-Siemens syndrome,” “Bloch-Sulzberger disease,” “Bloch-Sulzberger syndrome” “melanoblastosis cutis,” and “naevus pigmentosus systematicus”), galactosemia Microcephaly, alpha-1 antitrypsin deficiency (Alpha-1), Adenosine deaminase (ADA) deficiency, Severe Combined Immunodeficiency (SCID), neurofibromatosis type 1 (NF1), Wiskott-Aldrich syndrome, Stargardt macular degeneration, Fanconi's anemia, Spinal muscular atrophy (SMA) and Leber's congenital amaurosis (LCA).

In other embodiments non-hereditary diseases such as autoimmune diseases are particularly applicable for curing via knockout or downregulation of the autoantigen by using the method or system of the invention.

In some specific embodiments, the methods of the invention may be applicable for treatment and/or curing of RP.

Retinitis pigmentosa (RP) is an inherited dystrophic or degenerative disease of the retina with a prevalence of roughly one in 4,000. Typically, the disease progresses from the midperiphery of the retina into the central retina and, in many cases, into the macula and fovea. Clinical features include night blindness starting in adolescence, followed by progressive loss of peripheral vision, referred to as “tunnel vision”, culminating in legal blindness or complete blindness in adulthood. Characteristic retinal findings on examination include bone-spicule formations and attenuated blood vessels, reduced visual fields, reduced and/or abnormal electroretinograms (ERGs), changes in structure imaged by optical coherence tomography (OCT), and subjective changes in visual function. However, features and findings are highly variable among patients, even among patients within the same family. Currently, there are no effective treatments for RP. The invention is applicable for all modes of inheritance are encountered, specifically, dominant, recessive, autosomal, X-linked, and even mitochondrial, adRP accounts for 25%-30% of the cases.

It is assumed that each patient has a monogenic form of disease (or digenic in rare cases), but many different genes account for disease in RP patients as a group.

Finding genes and mutations causing adRP: for autosomal dominant diseases, the problems are compounded by the need to detect a single, heterozygous mutation in a diploid organism, the proverbial needle-in a-haystack. Clinical evaluation, NGS, segregation testing and linkage analysis are performed. It should be noted that the prevalence of adRP is around 1:15,000.

An accurate diagnosis of retinitis pigmentosa relies on the documentation of the progressive loss photoreceptor cell function, confirmed by a combination of visual field and visual acuity tests, fundus and optical coherence imagery, and electroretinography (ERG). The patient's family history is also considered due to the mode of inheritance. Clinical findings include night blindness or nyctalopia, Tunnel vision (due to loss of peripheral vision), Latticework vision, Photopsia (blinking/shimmering lights), Photophobia (Aversion to glare), Development of bone spicules in the fundus, Slow adjustment from dark to light environments and vice versa, Blurring of vision, Poor color separation, Loss of central vision and Eventual blindness.

In yet some further embodiments, the methods of the invention may be applicable for treating and curing PSACH. Pseudoachondroplasia (PSACH) is a skeletal dysplasia characterized by disproportionate short stature, small hands and feet, abnormal joints and early onset osteoarthritis. PSACH is caused by mutations in thrombospondin 5 (TSP-5, also known as cartilage oligomeric matrix protein or COMP), a pentameric extracellular matrix protein primarily expressed in chondrocytes and musculoskeletal tissues. The thrombospondin gene family is composed of matricellular proteins that associate with the extracellular matrix (ECM) and regulate processes in the matrix. Mutations in COMP interfere with calcium-binding, protein conformation and export to the extracellular matrix, resulting in inappropriate intracellular COMP retention. This accumulation of misfolded protein is cytotoxic and triggers premature death of chondrocytes during linear bone growth, leading to shortened long bones. Both in vitro and in vivo models have been employed to study the molecular processes underlying development of the PSACH pathology.

While PSACH is a rare disorder with an estimated birth prevalence of approximately 1/30,000 (www.orpha.net), the exact birth prevalence is not known since PSACH newborns are indistinguishable from other babies at birth.

PSACH is an autosomal dominant disorder that occurs as a (de novo) new event in 70-80% of families with the remaining cases being inherited from an affected parent.

The diagnosis of pseudoachondroplasia can be made on the basis of clinical findings and radiographic features. Identification of a heterozygous pathogenic variant in COMP on molecular genetic testing establishes the diagnosis if clinical features are inconclusive. Pseudoachondroplasia is one of the most common skeletal dysplasias affecting all racial groups. However, no precise incidence figures are currently available.

Clinical findings include: Normal length at birth, Normal facies, Waddling gait, recognized at the onset of walking, Decline in growth rate to below the standard growth curve by approximately age two years, leading to moderately severe disproportionate short-limb short stature, Moderate brachydactyly, Ligamentous laxity and joint hyperextensibility, particularly in the hands, knees, and ankles, Mild myopathy reported for some individuals, Restricted extension at the elbows and hips, Valgus, varus, or windswept deformity of the lower limbs, Mild scoliosis, Lumbar lordosis (˜50% of affected individuals), Joint pain during childhood, particularly in the large joints of the lower extremities; may be the presenting symptom in mildly affected individuals. Radiographic features include: Delayed epiphyseal ossification with irregular epiphyses and metaphyses of the long bones (consistent), Small capital femoral epiphyses, short femoral necks, and irregular, flared metaphyseal borders; small pelvis and poorly modeled acetabulae with irregular margins that may be sclerotic, especially in older individuals, Significant brachydactyly; short metacarpals and phalanges that show small or cone shaped epiphyses and irregular metaphyses; small, irregular carpal bones, Anterior beaking or tonguing of the vertebral bodies on lateral view. This distinctive appearance of the vertebrae normalizes with age, emphasizing the importance of obtaining in childhood the radiographs to be used in diagnosis.

In some specific embodiments, the methods of the invention may be applicable for treating and curing an MPO-related condition. In some embodiments, the MPO-related condition may be an immune-related disorder. An “Immune-related disorder” or “Immune-mediated disorder”, as used herein encompasses any condition that is associated with the immune system of a subject, more specifically through inhibition or the activation of the immune system, or that can be treated, prevented or ameliorated by reducing degradation of a certain component of the immune response in a subject, such as the adaptive or innate immune response. An immune-related disorder may include infectious condition (e.g., viral infections), metabolic disorders, auto-immune disorders, vasculitis, inflammation and proliferative disorders, specifically, cancer. In some embodiments, the immune-related disorder may be an autoimmune disease. In accordance with some embodiments, the methods of the invention are applicable in treating autoimmune disorders. An autoimmune disease is a condition arising from an abnormal immune response to a normal body part. Examples of an autoimmune disorder include Rheumatoid arthritis (RA), Multiple sclerosis (MS), Systemic lupus erythematosus (lupus), Type 1 diabetes, Psoriasis/psoriatic arthritis, Inflammatory bowel disease including Crohn's disease and Ulcerative colitis, and Vasculitis.

In some specific embodiments, the methods of the invention may be particularly applicable for autoimmune disorder such as multiple sclerosis (MS), Anti-neutrophil cytoplasmic antibodies (ANCAs)-related disorder, and systemic lupus erythematosus (SLE).

In some embodiments, the methods of the invention may be applicable for the treatment of MS and any related conditions or symptoms associated therewith. The term “Multiple Sclerosis” (MS) as herein defined is a chronic inflammatory neurodegenerative disease of the central nervous system that destroys myelin, oligodendrocytes and axons. MS is the most common neurological disease among young adults, typically appearing between the ages of 20 and 40. The symptoms of MS vary, from the appearance of visual disturbance such as visual loss in one eye, double vision to muscle weakness fatigue, pain, numbness, stiffness and unsteadiness, loss of coordination and other symptoms such as tremors, dizziness, slurred speech, trouble swallowing, and emotional disturbances. As the disease progresses patients may lose their ambulation capabilities, may encounter cognitive decline, loss of self-managing of everyday activities and may become severely disabled and dependent.

MS symptoms develop because immune system elements attack the brain's cells, specifically, glia and/or neurons, and damage the protective myelin sheath of axons. The areas in which these attacks occur are called lesions that disrupt the transmission of messages through the brain. Multiple sclerosis is classified into four types, characterized by disease progression: (1) Relapsing-remitting MS (RRMS), which is characterized by relapse (attacks of symptom flare-ups) followed by remission (periods of stabilization and possible recovery; while in some remissions there is full recovery, in other remissions there is partial or no recovery). Symptoms of RRMS may vary from mild to severe, and relapses may last for days or months. More than 80 percent of people who have MS begin with relapsing-remitting cycles; (2) Secondary-progressive MS (SPMS) develops in people who have relapsing-remitting MS. In SPMS, relapses may occur, but there is no remission (stabilization) for a meaningful period of time and the disability progressively worsens; (3) Primary-progressive MS (PPMS), which progresses slowly and steadily from its onset and accounts for less than 20 percent of MS cases. There are no periods of remission, and symptoms generally do not decrease in intensity; and (4) Progressive-relapsing MS (PRMS). In this type of MS, people experience both steadily worsening symptoms and attacks during periods of remission. It should be understood that the method of the invention may be applicable for any type, stage or condition of the MS patient. Treatment using the methods of the invention may result in some embodiments in alleviation of any symptoms, and/or in prolonging the remission period between attacks.

In yet some further embodiments, the methods of the invention may be applicable for the treatment of SLE, and any related conditions or symptoms associated therewith. More specifically, Systemic lupus erythematosus (SLE), also known simply as lupus, is an autoimmune disease. Symptoms vary between people and may be mild to severe. Common symptoms include painful and swollen joints, fever, chest pain, hair loss, mouth ulcers, swollen lymph nodes, feeling tired, and a red rash which is most commonly on the face. The disease is characterized by periods of illness, called flares, and periods of remission during which there are few symptoms.

The cause of SLE is not clear, however, is thought to involve genetics together with environmental factors. There are a number of other types of lupus erythematosus including discoid lupus erythematosus, neonatal lupus, and subacute cutaneous lupus erythematosus. It should be appreciated that the invention encompasses each of these types.

Still further, in some embodiments, the methods of the invention may be relevant for other auto immune disorders. For example, for the treatment of ANCA-associated disorders. Anti-neutrophil cytoplasmic antibodies (ANCAs), as used herein, include the perinuclear anti-neutrophil cytoplasmic antibodies (P-ANCA) that target mostly the MPO or EGPA, and are therefore also known as MPO-ANCA, Cytoplasmic anti-neutrophil cytoplasmic antibodies (c-ANCAs), that mostly target the proteinase 3 (PR3) protein and therefore are also known as PR3-ANCA, which is mostly associated with GPA, and atypical ANCA (a-ANCA), also known as x-ANCA, and are a group of autoantibodies, mainly of the IgG type, directed against antigens in the cytoplasm of neutrophil granulocytes (the most common type of white blood cell) and monocytes. p-ANCA is also associated with several medical conditions, it is fairly specific, but not sensitive for ulcerative colitis; a majority of primary sclerosing cholangitis; focal necrotizing and crescentic glomerulonephritis; and rheumatoid arthritis.

In some embodiments, the methods of the invention may be applicable for any ANCA-related or associated disorders. More specifically, such disorders include, but are not limited to ANCA-associated vasculitides (AAV), ANCA-associated glomerulonephritis (AAGN), crescentic glomerulonephritis (NCGN), and Rapidly progressive glomerulonephritis (RPGN).

In some further embodiments, the methods of the invention may be applicable for treating immune-related disorder such as an inflammatory disorder. In accordance with some embodiments, the methods of the invention are applicable in treating an inflammatory disorder. The terms “inflammatory disease” or “inflammatory-associated condition” refers to any disease or pathologically condition which can benefit from the reduction of at least one inflammatory parameter, for example, induction of an inflammatory cytokine such as IFN-gamma and IL-2 and reduction in IL-6 levels. The condition may be caused (primarily) from inflammation, or inflammation may be one of the manifestations of the diseases caused by another physiological cause. In some embodiments, an inflammatory disease that may be applicable for the methods of the invention may be any one of atherosclerosis, Rheumatoid arthritis (RA) and inflammatory bowel disease (IBD).

In yet some further embodiments, the MPO-related condition may be a neurodegenerative disorder. In some specific embodiments, the methods of the invention are applicable in treating a neurodegenerative disorder. In some embodiments, the neurodegenerative disorder may further involve inflammatory and/or vascular causes. Neurodegeneration is the umbrella term for the progressive loss of structure or function of neurons, including synaptic dysfunction and death of neurons. Many neurodegenerative diseases including Alzheimer's and Parkinson's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present invention.

In more specific embodiments, the methods of the invention may be applicable for treating a neurodegenerative disorder such as Alzheimer's disease or Parkinson's disease. Alzheimer's disease (AD), as used herein refers to a disorder that involves deterioration of memory and other cognitive domains that in general leads to death within 3 to 9 years after diagnosis. The principal risk factor for Alzheimer's disease is age. The incidence of the disease doubles every 5 years after 65 years of age. Up to 5% of people with the disease have early onset AD (also known as younger onset), that may appear at 40 or 50 years of age. Many molecular lesions have been detected in Alzheimer's disease, but the overarching theme to emerge from the data is that an accumulation of misfolded proteins in the aging brain results in oxidative and inflammatory damage, which in turn leads to energy failure and synaptic dysfunction. More specifically, accumulation of A1 within has been shown in structurally damaged mitochondria isolated from the brains of patients with Alzheimer's disease.

Alzheimer's disease may be primarily a disorder of synaptic failure. Hippocampal synapses begin to decline in patients with mild cognitive impairment (a limited cognitive deficit often preceding dementia) in whom remaining synaptic profiles show compensatory increases in size. In mild Alzheimer's disease, there is a reduction of about 25% in the presynaptic vesicle protein synaptophysin. With advancing disease, synapses are disproportionately lost relative to neurons, and this loss is the best correlate with dementia. Aging itself causes synaptic loss, which particularly affects the dentate region of the hippocampus.

Still further, in some embodiments, the target sequence targeted by the HDR enhancing gene editing systems provided by the invention may be any sequence encoding receptors for antigen derived from a pathogen specifically, viral, bacterial, fungal, parasitic pathogen and the like. Thus, in some embodiments, the therapeutic methods of the invention may be applicable for any condition caused by at least one pathogen. More specifically, any immune-related disorder or condition that may be a pathologic condition caused by any of the pathogens disclosed by the invention, for example, an infectious disease caused by a pathogenic agent, specifically, a viral, bacterial, fungal, parasitic pathogen and the like. Pathogenic agents include prokaryotic microorganisms, lower eukaryotic microorganisms, complex eukaryotic organisms, viruses, fungi, prions, parasites, yeasts, toxins and venoms. Still further, in some embodiments, the methods of the invention may be applicable for disorders caused by a viral pathogen. A viral pathogen, as used herein, may be in some embodiments, of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages). In yet some more specific embodiments, the methods of the invention may be applicable for a viral disorder such as a Foot and Mouth Disease.

It should be appreciated that the methods of the invention enable in vivo editing of a target nucleic acid sequence of interest in cells of the treated subjects, by administering to the treated subject the HDR enhancing nucleic acid guided genome modifier chimeric or fusion protein of the present disclosure, systems and/or any nucleic acid molecules encoding the nucleic acid guided genome modifier chimeric or fusion protein of the present disclosure. However, in some alternative embodiments, the desired editing of the target nucleic acid sequence, may be performed ex vivo. In such option, the editing, or genetic manipulation of the nucleic acid sequence of interest is performed in cells of an autologous or allogeneic source, that are then administered to the subject.

Thus, in some embodiments, the methods of the invention may involve the step of administering to the treated subject an effective amount of a cell that comprises the HDR enhancing nucleic acid guided genome modifier chimeric or fusion protein of the present disclosure, or a cell that has been modified by the modifier of the invention and any fusion protein modifier or effector thereof. In some embodiments, such cell has been ex vivo modified using the systems of the invention. Thus, in some embodiments thereof, the methods of the invention may comprise the step of administering to the treated subject a therapeutically effective amount of at least one cell as defined by the invention or of any composition comprising any of the cells disclosed by the invention.

Still further, in some embodiments, the cells may be of an autologous or allogeneic source.

In some embodiments, the “host cells” provided herein, specifically, the cells transduced, transfected with, and/or modified by, and/or comprising the HDR enhancing nucleic acid guided genome modifier chimeric or fusion protein of the present disclosure, and any systems thereof, and/or the encoding nucleic acid molecules provided by the invention, may be cells of an autologous source. The term “autologous” when relating to the source of cells, refers to cells derived or transferred from the same subject that is to be treated by the method of the invention.

In yet some further embodiments, the cells transduced or transfected with the nucleic acid guided genome modifier chimeric or fusion protein of the present disclosure, systems and/or nucleic acid molecules of the invention used by the methods of the invention may be cells of an allogeneic source, or even of a syngeneic source.

The term “allogeneic” when relating to the source of cells, refers to cells derived or transferred from a different subject, referred to herein as a donor, of the same species. The term “syngeneic” when relating to the source of cells, refers to cells derived or transferred from a genetically identical, or sufficiently identical and immunologically compatible subject (e.g., an identical twin).

In a further aspect, the invention provides an effective amount of at least one of:

(a) a nucleic acid guided genome modifier chimeric or fusion protein, complex or conjugate having enhanced homology-directed repair, or at least one nucleic acid sequence encoding said nucleic acid guided genome modifier chimeric or fusion protein. The nucleic acid guided genome modifier chimeric or fusion protein used herein, comprises: (i) at least one CRISPR-dCas protein devoid of a nucleolytic activity or any variant or mutant thereof; and (ii) at least one nucleic acid modifier component. The genome modifier chimeric or fusion protein used herein further comprises at least one of: (iii) at least one DAD for binding at least one Donor nucleic acid molecule; and/or (iv) at least one RFRD. Still further, in some embodiments (b), at least one donor nucleic acid molecule may be further used. In some embodiments, the donor molecule comprises at least one replacement sequence for incorporation in, and/or for replacing, a target nucleic acid sequence of interest in the genome of the treated subject. In some embodiments, such target sequence is associated directly or indirectly with the treated disorder. In yet some further embodiments (c), at least one target recognition element or any nucleic acid sequence encoding said target recognition element may be used herein. In some embodiments, the target recognition element specifically recognizes and binds the target sequence in the genome of at least one cell of the treated subject. Still further, in some embodiments (d), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c), may be used. In yet some further embodiments (e), at least one system comprising (a) and at least one of (b) and (c). Still further alternative embodiments of the disclosed use pertain to (f), at least one cell comprising and/or modified by, at least one of: (a), (b), (c), (d) and (e). Finally, in some embodiments (g), at least one composition comprising at least one of (a), (b), (c), (d), (e) and (f) may be used. More specifically, the effective amount of the disclosed elements (a) to (g) may be for use in method of curing or treating, preventing, inhibiting, reducing, eliminating, protecting or delaying the onset of a pathologic disorder or condition in a subject in need thereof.

In some embodiments, the CRISPR-dCas protein suitable for the nucleic acid guided genome modifier chimeric protein, complex or conjugate used by the methods of the present disclosure may be at least one of Cas9, CasX, Cas12a1, CasF, Cas14a1, an ancestral Cas and Cas14b5. Still further, in some embodiments, such CRISPR-dCas protein has reduced or abolished PAM constraint. In such case, at least one of the PBD, any fragment of the PBD, and at least one amino acid residue adjacent to the PBD of such CRISPR-dCas protein, may be deleted or replaced.

In some other embodiments, the nucleic acid guided genome modifier chimeric protein, complex or conjugate for use according to the invention, the nucleic acid molecule for use according to the invention, the system for use according to the invention, the host cell for use according to the invention and the composition for use according to the invention may be as defined herein before in connection with other aspects of the invention.

In another aspect, the invention provides an effective amount of at least one of:

(a) a nucleic acid guided genome modifier chimeric or fusion protein, complex or conjugate having enhanced homology-directed repair or at least one nucleic acid sequence encoding said nucleic acid guided genome modifier chimeric or fusion protein. The nucleic acid guided genome modifier chimeric or fusion protein used herein, comprises: (i) at least one CRISPR-dCas protein devoid of a nucleolytic activity or any variant or mutant thereof; and (ii) at least one nucleic acid modifier component. The genome modifier chimeric or fusion protein used herein further comprises at least one of: (iii) at least one DAD for binding at least one Donor nucleic acid molecule; and/or (iv) at least one RFRD. Still further, in some embodiments (b), at least one donor nucleic acid molecule may be used as disclosed herein. In some embodiments, the donor molecule comprises at least one replacement sequence for incorporation in, and/or for replacing, the target nucleic acid sequence of interest. In some further embodiments (c), at least one target recognition element or any nucleic acid sequence encoding the target recognition element may be further used. In some embodiments, the target recognition element specifically recognizes and binds the target sequence. In yet some alternative embodiments (d), at least one nucleic acid cassette or any vector or vehicle comprising at least one of: (i) the nucleic acid sequence of (a); (ii) the nucleic acid sequence of (b); (iii) the nucleic acid sequence of (c); and (iv) the nucleic acid sequence of (a) and at least one of (b) and (c). Still further, in some embodiments (e), at least one system comprising (a), (b), (c) and (d). In yet some further embodiments (f), at least one composition comprising at least one of (a), (b), (c), (d) and (e), according to this aspect, an effective amount of the disclosed elements (a) to (f), are for use in method of modifying at least one target nucleic acid sequence of interest in at least one cell.

In some embodiments, the CRISPR-dCas protein suitable for the nucleic acid guided genome modifier chimeric protein, complex or conjugate used by the methods of the present disclosure may be at least one of Cas9, CasX, Cas12a1, CasF, Cas14a1, an ancestral Cas and Cas14b5. Still further, in some embodiments, such CRISPR-dCas protein has reduced or abolished PAM constraint. In such case, at least one of the PBD, any fragment of the PBD, and at least one amino acid residue adjacent to the PBD of such CRISPR-dCas protein, may be deleted or replaced.

In some other embodiments, the nucleic acid guided genome modifier chimeric protein, complex or conjugate for use according to the invention, the nucleic acid molecule for use according to the invention, the system for use according to the invention, the host cell for use according to the invention and the composition for use according to the invention may be as defined herein before in connection with other aspects of the invention.

An “effective amount” of the HDR enhanced nucleic acid guided genome modifier chimeric or fusion protein of the present disclosure, systems, nucleic acids, host cells of the invention comprised within any of the compositions disclosed herein, can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

It is to be understood that the terms “treat”, “treating”, “treatment” or forms thereof, as used herein, mean curing, preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation”, “prevention”, “suppression”, “repression”, “elimination” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention.

As indicated above, the methods and compositions provided by the present invention may be used for the treatment of a “pathological disorder”, specifically, immune-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understand to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprise”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### Cell Lines

HEK-293 cells [Embryonic Kidney; Human (Homo sapiens), ATCC].

### Primary Cells

Human blood-derived immune cells were extracted by immunodensity negative selection from fresh whole blood according to the protocol provided using RosetteSep Human T Cell Enrichment Cocktail (Stemcell USA) except that 25 μl of antibody cocktail was used for 15 ml of blood and that PBS was supplemented with Human Albumin (HSA) instead of FBS. Cells were resuspended to 3×10\hat{ }6/ml in full RPMI. T-cells were then activated by seeding 3 ml/well in 6-well activation plates (activated plates prepared by overnight incubation at 4 C with 2 ml/well of PBS with 1.1 ug/ml CD3 antibody and 1.1 ug/ml CD28 antibody, blocked with 1% HSA and incubation at 37 C for 20 minutes after which they were frozen at −20 C. Prior to use plates are defrosted, emptied and washed twice with PBS). Three days after activation cells were pooled, counted and resuspended in Lonza P3 buffer immediately before electroporation to 1×10\hat{ }6 cells/20 ul.

Directly after electroporation 0.5×10\hat{ }6 cells in 50 μl were transferred from the cuvette in duplicate to round bottom 96-well plates containing 150 μl prewarmed RPMI with 200 U/ml IL2 (with or without HDR enhancer [IDT]).

Medium containing HDR enhancer was removed after overnight incubation and replaced by 200 μl full RPMI with IL2.

24 hrs after electroporation GFP in control treatments was visually assessed to estimate electroporation efficiency.

4 days after electroporation cells were reactivated. 150 μl of medium was removed and replaced by 150 μl activation medium (full RPMI with 1000 U/ml IL2 and 12.5 ul/ml of CD3/CD28 activation beads) and cells were then resuspended.

7 days after electroporation duplicates were pooled into a 48-well flat-bottom plate and 400 μl of fresh full RPMI with 1000 U/ml of IL2 was added.

9-10 days from electroporation volume was measured, 10 μl were counted, 70 μl were taken to FACS analysis and the remaining 0.6-2.8×10\hat{ }6 cells were lysed for gDNA extraction.

### Constructs

Plasmids for the expression of the modified Cas constructs were constructed by Gibson assembly of a DNA fragment (ordered from GeneArt) into a KanR PUC57-based backbone under the CMV promotor and the BGH terminator.

mRNA In Vitro Transcription (IVT) Capped and tailed mRNA expressing each nuclease or control GFP mRNA was transcribed in vitro from plasmids harboring a T7 promoter (for GFP) or from a purified PCR product amplified from plasmids encoding human codon optimized plasmids (for nucleases) using the following protocol.

### 1) PCR FOR IVT

A hi-fidelity PCR product was prepared as a template for In Vitro Transcription (IVT). The forward primer encompasses a T7 promoter.

### Amplification

Q5 high fidelity DNA polymerase (NEB M0491S) was used to amplify the template. To get high concentrations of DNA several 50 μl PCR reactions were pooled.

TABLE 1a

| COMPONENT | 50 μL reaction |
|---|---|
| 5X Q5 buffer | 10 μl |
| 10 mM dNTPs | 1 μl |
| 10 mM T7 primer F | 2.5 μl |
| 10 mM primer R | 2.5 μl |
| Template Plasmid | 2 μl |
| (diluted) | |
| Q5 high fidelity | 0.5 μl |
| DNA polymerase | |
| Nuclease free water | 31.5 μl |

Assemble on ice. Preheat thermocycler to 98° C.

TABLE 1b

| Step | Temp | Time |
|---|---|---|
| Initial denaturation | 98° C. | 30 sec |
| 30 cycles | 98° C. | 5-10 sec |
| | 64° C. | 10-30 sec |
| | 72° C. | 3 min |
| Final | 72° C. | 2 min |

### Purification

1) PCR products were purified for next step using a PCR cleanup kit (Zymo D4003).
2) Elution in 12 μl pre-warmed buffer supplied in the kit (10 mM Tris-HCl, pH 8.5, 0.1 mM EDTA) or the same without EDTA making sure no nucleases are introduced.
3) Expect ˜2-3 μg/50 μl PCR rxn. For the next step about 1 μg in less than 6.3 μl are needed.
4) DNA concentration was measured after purification and a 1 μl sample was analyzed on gel.

### 2) IVT

IVT with this protocol results in high yields of pure capped, tailed mRNA ready for electroporation or other means of transfection. Yield is typically ˜60 μg of mRNA from each μg of blunt PCR template. Using either the following full kit and instructions from CellScript, USA: T7 mScript™ Standard mRNA Production System (C-MSC100625) or alternatively assembled from components below according to their instructions except for the marked modifications:
1) RNA Polymerase: Use either T7 mScript from the kit or separate high yield T7 enzymes such as T7 FlashScribe
Incubating for 2.5 Hrs at 37 C instead of 30 minutes.
2) mRNA samples were analyzed after DNAse by nanodrop quantification and electrophoresis next to an RNA ladder.
3) Capping was conducted using ScriptCap™ Cap 1 Capping System (SCCS1710)
4) Tailing was conducted using Poly(A) Polymerase Tailing Kit (PAP5104H)
5) Capped and tailed mRNA samples were phenol/chloroform purified, precipitated with Ammonium acetate and resuspended in water, analyzed by nanodrop quantification and electrophoresis next to an RNA ladder.
   sgRNAs (SCNAs)

Synthetic guide RNAs were custom designed in-house and ordered from commercial vendors (IDT or Synthego) with default Cas9 scaffold and chemical modifications. PD1 exon1 guide sequences used in example 4 for paired nucleases were #3478 GUCUGGGCGGUGCUACAACU (SEQ ID NO: 178) together with #3479 CUGUGGGAUCUGCAUGCCUG (SEQ ID NO: 179) and for Cas9 #3480 GGCGCCCUGGCCAGUCGUCU (SEQ ID NO: 180). PAM was set at NGG for Cas9 and at NNG for dimeric dscCas9-derived nucleases.

### Reverse Transfection

Reverse transfection to HEK-293 cells was done using TransIT® LT1 transfection reagent (Mirus Bio) diluted in serum-free medium Opti-MEM® I (Gibco). Transfection reagent:DNA ratio of 5 μl per 1 μg DNA was used. For reverse transfection in a well in 96 wells plate: 100 ng plasmid DNA was used in 3 μl total volume (33.3 ng/ul). Transfection reagent was first diluted (1:19) in Opti-MEM® I. Then, 3 μl DNA (33.3 ng/ul) was added to the diluted transfect reagent, mixed gently and incubated 15-30 min in room temperature. Then, HEK-293 cells (3.6×10⁴) were gently added on the top of the TransIT®-DNA complexes and mixed as is customary. Cells were incubated 72 h in 37° C. in a CO2 incubator (HERACELL 150i, Thermo Scientific). If co-transfection was done, plasmids were equally mixed in advance to final DNA concentration of 100 ng/3 μl. (For example: if two plasmids were used, 50 ng from each plasmid was mixed in 3 μl final volume 33.3 ng/μl DNA, if three plasmids were used, 33.3 ng from each was mixed in 3 μl final volume 33.3 ng/μl DNA).

### Electroporation

Activated primary human T-cells or Hek293 cells were electroporated using the Lonza 4D Nucleofector with 16-well cuvette strips using the following protocol:
A. Prepare in 8-well strips RNA+donor DNA. Mix on ice in as small volume as possible (maximum 7.5 μl) in the following order:
I. Combine 3 g capped and tailed mRNA of nuclease (or control) plus 5 g RNA from each guide (13 g RNA total for dimeric nucleases or Sg for Cas9).
II. Add PGA15-50 kDa 100 g/g 10.5 μl and RNase inhibitor 50 U/μl 0.11.
III. Add Donor DNA 0.5 μg.
B. Turn on Lonza 4D-Nucleofector and choose cuvette type and places and program EH115 for T-cells or program CM-130 for Hek293. Refrigerate cuvettes.
C. For T-cells Incubate 80 μl pre-warmed Full RPMI (with 200 U/ml IL2) per each treatment (1.5-2 ml for 16 treatments) in 37° C. For Hek293 add prewarmed full DMEM.
D. For T-cells pre-warm 96-well culture plates (round bottom) with Full RPMI (with 200 U/ml IL2), 150 μl/well. 2 wells per electroporation treatment. For Hek293 prewarm 24-well flat-bottom plates with DMEM. HDR enhancer V2 (IDT) stock: 0.69 mM. Concentration in medium is 1.5 μM (2.17 μl/ml). Concentration is calculated for the 150 μl (before addition of cells). For each treatment 150 μl medium+0.325 μl/well. Thus, for 16 wells (+spare)=2.5 ml medium+5.4 μl HDR enhancer.
E. Prepare cells—For T-cells 1×10\hat{ }6 activated (day 3 from activation on CD3/CD28 plates) T-cells for each treatment; For Hek293 2×10\hat{ }5 cells per treatment, trypsinyzed for 4 minutes and then resuspended in DMEM:
I. Anti-CD3 and anti-CD28 stimulated primary human T cells or Hek293 cells are resuspended, pooled and washed twice in sterile PBS at 100×g for 10 minutes (500×g for 5 minutes for Hek293), RT (For T-cells first wash 50 ml, second wash 15 ml).
II. Count between washes: Take 10 μl cells plus 10 μl trypan blue and count cells in cell counter (trypan blue program. Clean with kimwipes between counts. Cells viability should be higher than 85% (better higher than 90%). For 16 treatments (one cuvette strip) take 17 million T-cells into 15 ml tube or 3.4 million Hek293 cells. Pellet @100×g for 10 minutes and remove all PBS. Place on ice.
III. About 1×10\hat{ }6 T-cells or 2×10\hat{ }5 Hek293 cells are gently resuspended in 20 l/ice-cold electroporation buffer, P3 for T-cells or SF for Hek293 (Lonza). (Prepare 340 μl electroporation buffer). The exposure time to the electroporation buffers is kept as short as possible. Creation of bubbles Should be avoided.
F. The next steps should be done promptly but gently:
I. Take 20 μl cells in electroporation buffer and add to the ˜5 μl RNA mix (on ice) that was prepared in step (A).
II. keeping the correct orientation is essential. Cells+RNA mix are carefully transferred onto the cold electroporation strips using 200 μl tips to avoid trapping air in the solution. Electroporation strips and cartridges are tapped onto the bench several times to ensure correct placement of fluids within the electroporation vessel.
III. Electroporation is performed on a 4D-Nucleofector™ Device (Lonza) using the program EH-115 for T-cells or program CM-130 for Hek293. Close the cover of the cuvette and transfer to electroporator. Press “START” for electroporation.
IV. Directly after electroporation transfer cuvette back to the cell culture hood and add sterile pre-warmed 80 μl medium (from step C) (total volume 80+˜25=˜105)
I. Resuspend and transfer 50 l/well of T-cells from cuvette to 96-well plate containing 150 μl prewarmed RPMI (with or without HDR enhancer) or total volume of cuvette in case of Hek293 into a single well in the 24-well plate from stage D. Final T-cell density/well=0.5×10\hat{ }6 cells (Two wells per treatment are used, position duplicates A1, A2 etc.) or 2×10\hat{ }5 for Hek293.
G. Incubate cells between 3 hrs and O/N at 37° C. and wash out HDR enhancer. For T-cells Centrifuge plate 10 minutes 100×g RT ° C. Add 200 μl new prewarmed Full RPMI with IL2/well. Medium is simply replaced for the adherent Hek293.

### Genomic DNA Preparation

Genomic DNA (gDNA) from HEK-293 cells was extracted 72 hours post transfection for NHEJ-ER experiments, and alternatively as indicated in specific cases, using the Quick-DNA™ 96 Kit (Zymo Research) and gDNA from T-cells was extracted 9-10 days post electroporation using the equivalent Quick-DNA™ miniprep Kit according to instructions. The concentration of gDNA was determined using NanoDrop 2000 (Thermo Scientific).

### Droplet Digital PCR Design and Execution

QX200™ Droplet Digital™ PCR system (BIORAD) was used for the mutations analysis in the target site on the gDNA.

Assay designed, TGEE3 or TGEE4 for mutation detection in EMX1 gene-Target 2: Detailed reaction particulars and primers can be found in Table 2 (Tables 2a, 2b, 2c and 2d). As TGEE3 and TGEE4 are assays to analyze mutation in adjacent sites, same primers were used for the amplification of the target site were in both assays. In addition, the same reference probe was used for both assays. The drop-off probes were different and specific for each site.

Primers 3042-Forward and 3049-Reverse (as denoted by SEQ ID NO. 24 and SEQ ID NO. 25, respectively), were designed to amplify one specific 317 bp fragment in Tm=55° C. The primers that were designed as reference and drop off probes were first tested in a simple PCR reaction to amplify a specific fragment when using with the 3049-Reverse primer in Tm=55° C. Then, the reference probe was ordered from IDT with FAM™ modification in the 5′ end and with Iowa Black® Quencher in the 3′ end. The drop off probe was ordered from IDT with HEX™ modification in the 5′ end and with Iowa Black® Quencher in the 3′ end with 2 locked nucleic acid (LNA) bases inside the target site. Tm of reference and drop off probes were designed to be higher in 3-10° C. than 55° C.

TABLE 2a
Assay execution:

| EMX1-Target1-TGEE3 | |
|---|---|
| orTGEE4assay | for 1 reaction |
| 2XddPCR Supermix for probes | 11 μl |
| 20X NHEJ TGEE-3 or TGEE4 | 1 μl |
| EMX1-T1 | |
| HindIII diluted 2-5 U/μl | 1 μl |
| 150 ng gDNA | 9 μl |

TABLE 2b

| 20X NHEJ TGEE-3 or TGEE4 | μl for 100 |
|---|---|
| EMX1-T1 | reactions |
| 18 μM primer 3042-EMX1-T1- | 18 μl |
| ddPCR-F1 | |
| 18 μM primer 3049-EMX1-T1- | 18 μl |
| ddPCR-R2 | |
| 100 μM 3088-EMX1-T1-TGEE3- | 5 μl |
| reference probe | |
| 100 μM 3089-EMX1-T1-TGEE3- | 5 μl |
| drop off probe (HEX/2XLNAs) or | |
| 13175-EMX1-T1-TGEE4-drop | |
| off probe (HEX/2XLNAs) | |
| DDW | 54 μl |

TABLE 2c

| | μl for 100 |
|---|---|
| HindIII diluted 2-5 U/μl | reactions |
| 10X buffer CutSmart NEB | 10 μl |
| HindIII NEB | 25 μl |
| DDW | 65 μl |

TABLE 2d

| Primers and probes | for 1 reaction |
|---|---|
| 3042-EMX1-T1-ddPCR-F1 | gaaaccatgcccattc as denoted by SEQ ID NO: 24 |
| 3049-EMX1-T1-ddPCR-R2 | agagaagggtggttttc as denoted by SEQ ID NO: 25 |
| 3088-EMX1-T1-TGEE3- | /56-FAM/CT CTG TAT G/ZEN/G AAA AGA GCA |
| reference probe | T/3IABkFQ/ as denoted by SEQ ID NO: 26 |
| 3089-EMX1-T1-TGEE3-drop off | /5HEX/tcgtaga+g+tcccatgtc/3IABkFQ as denoted by |
| probe | SEQ ID NO: 27 |
| 3175-EMX1-T1-TGEE4-drop off | /5HEX/ATGTCTGC+C+GGCTTCC/3IABKFQ |
| probe | as denoted by SEQ ID NO: 28 |

### Data Analysis

QX200™ Droplet Reader and QuantaSoft™ software (BIORAD) were used for Data analyzing.

For each assay and each experiment, a threshold was determined in relation to all experiment treatments, controls and no DNA control sample.

Thresholds were analyzed separately for each experiment and for each assay, as presented by Table 3:

TABLE 3
ddPCR Analysis thresholds

| Experiment | Assay TGEE3 | Assay TGEE4 |
|---|---|---|
| 1 (#13) | | FAM1840/HEX2410 |
| 2 (#16) | FAM 1500/HEX1430 | FAM1500/HEX1770 |
| 3 (#32) | | FAM 2900/HEX 1900 or |
| | | FAM2020/HEX1570 |

Poisson correction was done according to manufacturer's instructions (Droplet Digital PCR Applications Guide, BioRad, p7-8). Briefly, a Poisson correction factor is inferred by modeling a Poisson distribution from the fraction of empty cells. Explicitly, the Poisson correction factor is the infinite sum of the probability of a cell containing 1 DNA molecule only, plus two times the probability of two DNA molecules, plus three times the probability of three DNA molecules, and so on. This correction factor is multiplied with the observed number of hits to find the true number of DNA molecules.

### Example 1

### Cas Protein Comprising Donor Attachment Domain (DAD)

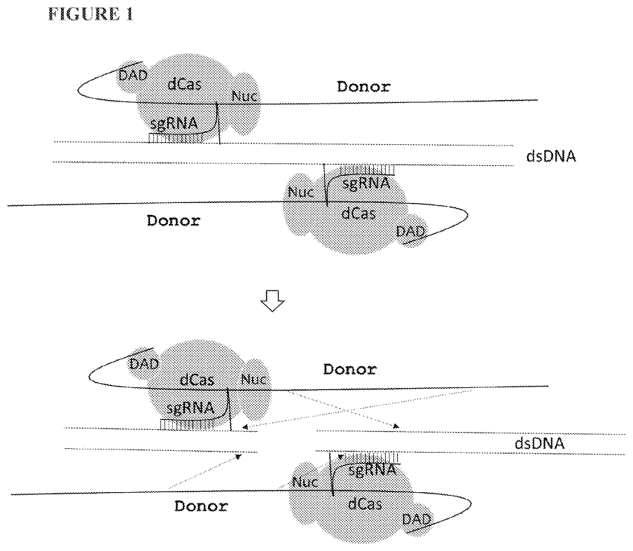

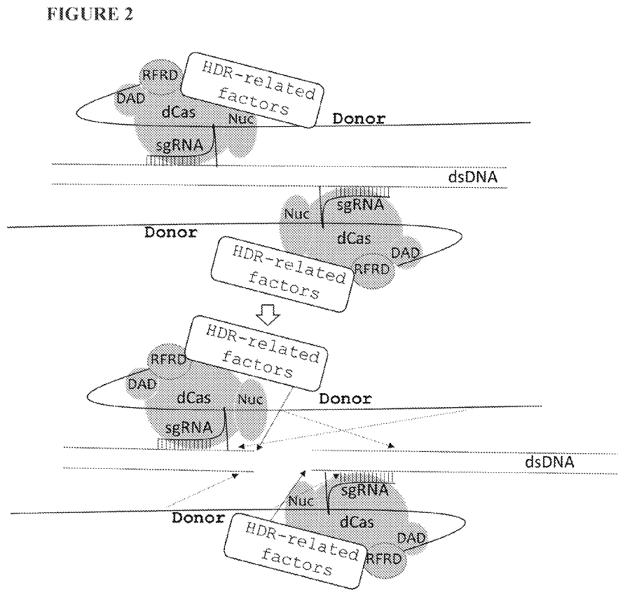

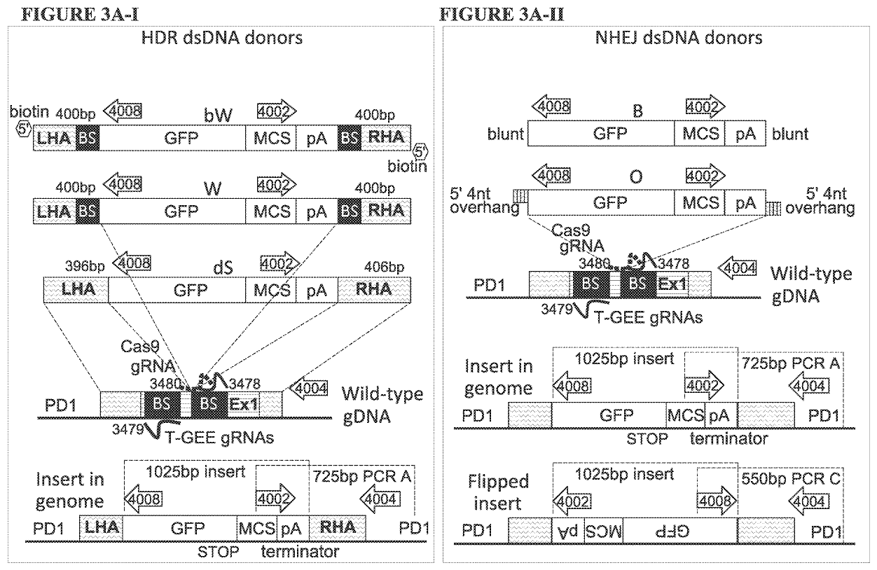

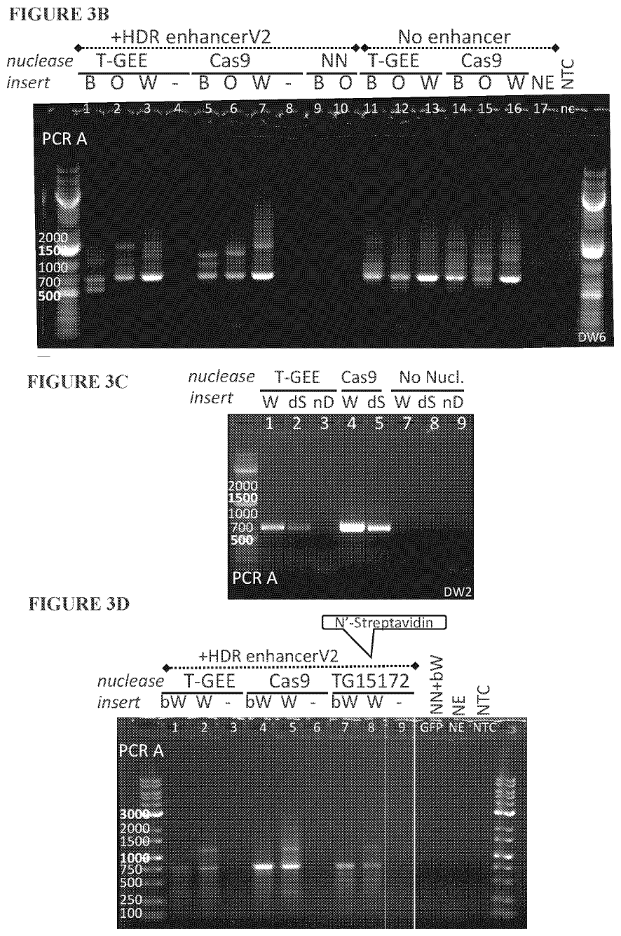

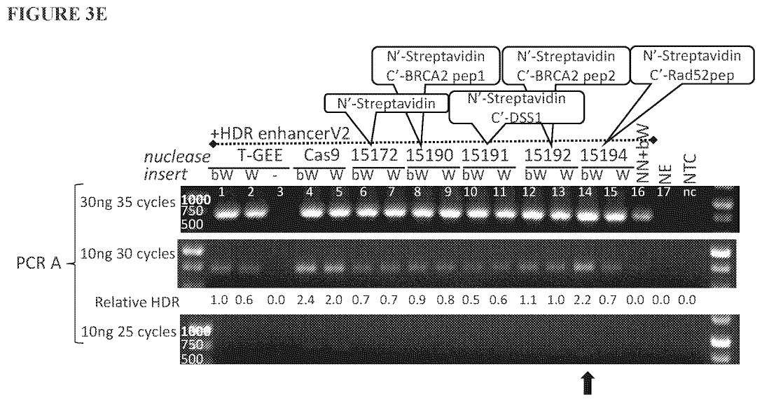

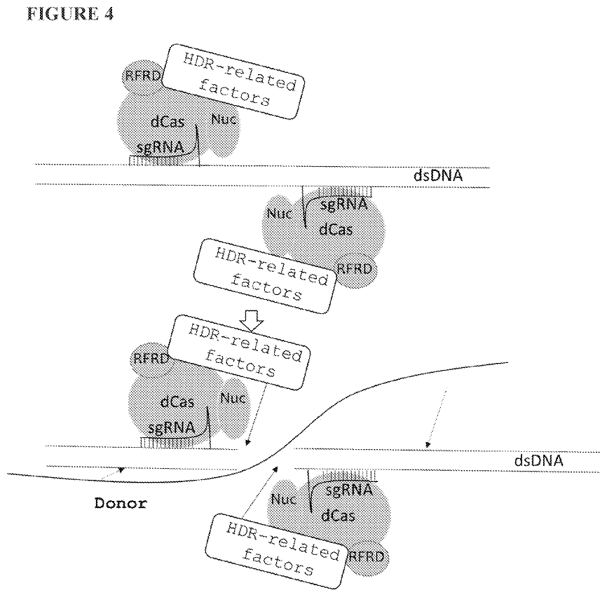

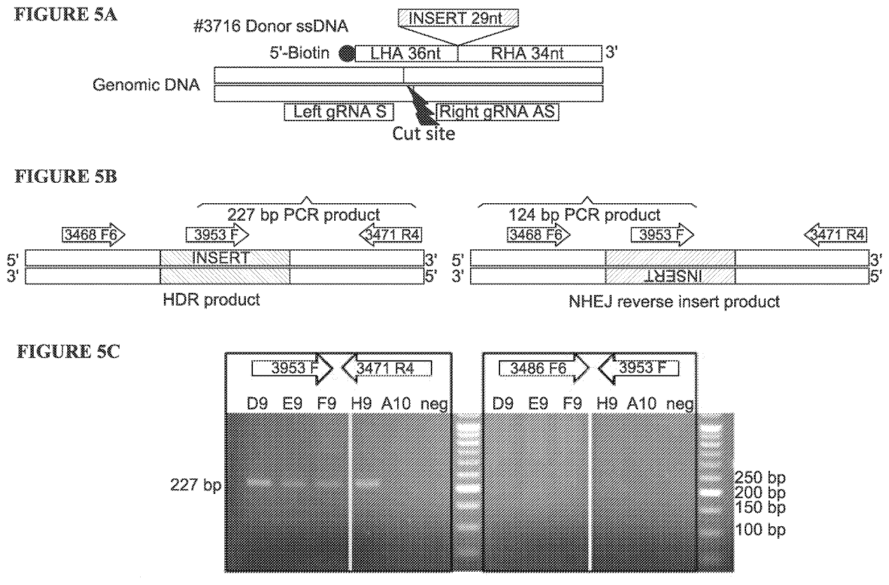

To provide improved systems for specific genome modifications, homology-directed repair may be used, whereby a Donor nucleic acid comprise sequences having homology to the target sequence (homology arms) that flank the sequences that are incorporated into the target DNA. The incorporation of the sequence into the target site is accomplished by homologous recombination following target DNA cleavage by a nuclease. Addition of HDR-enhancement domains to genome editing nucleases may improve their ability to induce HDR. Attachment of Donor nucleic acid to the RNA-guided nuclease by “donor attachment domains (DAD)” could potentially allow higher local concentration at the cleavage site and lower overall concentrations of nucleic acids in the transfected cell. As illustrated in FIGS. 1 and 2, the inventors next designed Cas proteins that comprise various DADs, that include sequence specific DADs, non-specific sequence DADs, and DADs that ae based on covalent interactions.

### Attachment Via Sequence Specific Donor Attachment Domain

As indicated above, attachment of Donor nucleic acid can be achieved using sequence-specific nucleic acid binding domains which bind to donor DNA with specific sequences (“sequence specific donor attachment domain”). As sequence specific DADs, the inventors constructed Cas9 proteins comprising the following sequence specific DADs: zinc fingers (SEQ ID NO: 1), lambda repressor DNA binding domain (SEQ ID NO: 2) (Stayrook et al, 2008, Nature, 452:1022-25), Gal4 DNA binding domain (Keegan et al, 1986, Science, 231:699-704), and Pot1 ssDNA binding domain (SEQ ID NO: 3) (Lei et al, 2003, Nature, 426:198-203).

### Attachment Via Covalent Interactions

Attachment of Donor nucleic acid sequence (also referred to herein as “Donors”) can also be achieved by covalent interactions including conjugation by a virD2 domain (SEQ ID NO: 4), an endonuclease that can cleave and covalently attach to the 5′ end of single-stranded DNA with a defined sequence (Young and Nester, 1988, Journal of Bacteriology, 170, 3367-74; Shiboleth and Weinthal, 2015, Compositions and Methods for Modifying a Predetermined Target Nucleic Acid Sequence). The inventors therefore next constructed Cas9 protein comprising a virD2 domain.

### Attachment Via Non-Sequence Specific Donor Attachment Domains

Attachment to the donor can also be achieved by non-sequence specific domains (“non-sequence specific donor attachment domains”) that bind to chemical groups present on the donor DNA, such as a streptavidin domain that can bind to biotinylated donor DNA (SEQ ID NO: 5) (Shiboleth and Weinthal, 2015, Compositions and Methods for Modifying a Predetermined Target Nucleic Acid Sequence). The inventors therefore next constructed Cas9 protein comprising a streptavidin domain.

To test HDR vs NHEJ mediated insertion in HEK293 and T-cells the inventors constructed a set of donors (FIG. 3A-I, 3A-II, for HDR and NHEJ, respectively). This set of donors when correctly inserted into the human PDCD1 (PD1) exon 1 locus would allow expression of GFP from the endogenous PD1 promoter and abolish PD1 protein, together, a functional gene replacement. An exception is the Blunt (B) NHEJ donor denoted by SEQ ID NO:181 (1053 bp) which is inserted out-of-frame. Overhang (O) NHEJ donor has an identical slightly shorter sequence (1029 bp) derived from donor B after digestion by BsaI from both ends. This results in four single strand nucleotides at the 5′ of each side, corresponding to the expected sticky overhang in PD1 exon1 at the nuclease cleavage site of the HDR enhanced nucleic acid guided genome modifier chimeric or fusion protein of the present disclosure, creating a seamless in-frame fusion. Cleavage of this gene with spCas9 does not create a compatible sticky site.

To first compare between blunt (B), overhang (O) and an HDR donor (W) (SEQ ID NO: 182) (FIG. 3B) an experiment was conducted in Hek293 cells. W denotes the wSCNA donor-with full SCNA (guide RNA) 3478 (SEQ ID NO: 178) and 3479 (SEQ ID NO: 179) binding sites (BS). In W the 3480 (SEQ ID NO: 180) Cas9 guide has only a 9 bp overlap+PAM within the Right Homology Arm (RHA) designed so it cannot cut the donor.

Results showed that in all treatments HDR resulted in properly recombined GFP in the PD1 locus, with a dominant product at the correct size as compared to the NHEJ donors B and O which had a much more “messy” outcome with probable concatamers and mis-inserted products (FIGS. 3B-3D). Analysis of flipped inserts using primers 4004 and 4008 (SEQ ID NOs: 184, 185, respectively) also confirmed that HDR donors resulted in much less mis-inserted donor than the NHEJ donors. Only minor differences for the HDR donor were observed between monomeric spCAS9 (Cas9) and dimeric T-GEE (the HDR enhanced nucleic acid guided genome modifier chimeric or fusion protein of the present disclosure). More specifically, T-GEE used in this example, denotes mRNA transcribed from TG14664 (SEQ ID NO: 173) which is an ancestral dCasFok, his-tagged, Cas9 denotes mRNA transcribed from TG7665-Cas9 (SEQ ID NO: 41) which comprises spCas9. HDR enhancer (Alt-R HDR Enhancer V2, IDT) had no significant effect in this cell line in this experiment (FIG. 3B).

To establish whether the SCNA binding sites (BS) on donor W had an effect on HDR efficiency a similar experiment (without HDR enhancer) in Hek293 was conducted (FIG. 3C). Donor dS (SEQ ID NO: 183) denotes the dSCNA donor-without SCNA binding sites whereby Cas9 guide 3480 has no BS, and the two T-GEE guides have truncated binding sites; 3479 (SEQ ID NO: 179) has an 11 bp overlap+PAM, and 3478 (SEQ ID NO: 178) has an 10 bp overlap+PAM. In this experiment it was observed that the BS may have a positive effect for both T-GEE and Cas9.

A 5′-biotin was added to donor W (same sequence as W) denoted as bW. As seen in FIG. 3D showing HDR directed replacement of PD1 by GFP in activated human T-cells, biotinylated dsDNA donor DNA (bW) may by itself have advantages related to reduced concatenation/ligation of donor molecules. See lanes 2 vs 1; 5 vs 4; 7 vs 8 and 11 vs 10 who all contain an additional oversized band when no biotin is present in the donor DNA (W); With construct TG15172 (Streptavidin (n-term), ancestral dCasFok, his-tagged, SEQ ID NO: 158) harboring the DAD N′-streptavidin domain it can be seen that donor bW (lane 7) has higher HDR efficiency than the same donor with the non-streptavidin fused nuclease (lane 1) ostensibly due to the possibility that the streptavidin binds the biotin on the donor dsDNA bringing the donor in proximity to the dsDNA break site. Strengthening this hypothesis is the observation that the non-biotinylated donor W does not exhibit this difference (compare lanes 2 and 8). To establish whether combinations of a DAD (Streptavidin) and different RFRDs (BRCA peptides 1 and 2, DSS1 and Rad52 pep) may have superior effects an experiment in human Hek293 cells (FIG. 3E) was performed. Cells were harvested 7 days after electroporation. As initially all treatments containing any nuclease had very high levels of HDR in this experiment the difference between the treatments could not be quantitated. The background level of HDR in absence of nuclease can be seen in lane 16 top panel, presumably also aided by the presence of HDR enhancer that was present in all treatments. Thus, the amount of input genomic DNA (gDNA) was reduced from 60 ng/PCR used in FIGS. 3B, 3C and 3D, first to 30 ng (top panel), then to 10 ng only (corresponding to about 1,600 cells), concomitantly reducing the number of cycles to 30 (middle panel) or to 25 cycles (bottom panel). Under the middle panel appear the relative intensity of each band as quantitated by NIH Image software. PCR C (not shown) was performed as a control with 30 ng gDNA and 35 cycles and resulted in no visible bands validating the uni-directionality and lack of concatenation of insertion expected by accurate HDR.

As can be observed in lane 14 the additive effects of a DAD (streptavidin) when presumably bound to the biotin on the bW donor DNA can improve the HDR efficiency of TG15194 (SEQ ID NO:172, comprising a Streptavidin (n-term), RAD52 peptide (c-term), ancestral dCasFok), which was here found to be 2.2 times higher than the non HDR-enhanced T-GEE (SEQ ID NO: 173), and quite similar to that of Cas9 (lanes 4,5). Importantly, this higher level of HDR was achieved even though this HDR enhanced nucleic acid guided genome modifier chimeric or fusion protein had relatively meager NHEJ-ER editing levels of 22-24% when compared to Cas9 which has double its NHEJ-ER efficiency (Table 6). This may be in line with the hypothesis that NHEJ-ER and HDR are competing processes of DNA repair and show how an appropriate RFRD may help in pushing repair in the direction of HDR. Strengthening the role of DAD involvement is the observation that compared to a donor without biotin (W, lane 15), TG15194 with the bW donor (lane 14) had 3-fold higher HDR levels.

Taken together, these results establish the feasibility of performing an effective HDR using the HDR enhanced nucleic acid guided genome modifier chimeric or fusion proteins of the present disclosure that contain DAD.

### Example 2

### HDR Enhancement by Recruitment of Cellular Genomic DNA Repair Factors

HDR can also be enhanced by use of protein domains that are able to recruit cellular genomic DNA repair factors (“repair factor recruitment domain” (RFRD)) as illustrated in FIGS. 2 and 4, including Rad51 and Rad52, which mediate homology-based repair. Rad51 may be recruited by peptides derived from BRCA2 (SEQ ID NO: 6) (Carreira et al., 2009, Cell, 136:1032-1043), peptides derived from RAD54 (SEQ ID NO: 29) (Goyal et al., 2018, Nature Communications, 9:34), and peptides derived from RAD52 (SEQ ID NO:30) (Shen et al., 1996, JBC, 271:148-152). Rad52 may be recruited by peptides derived from DSS1 (SEQ ID NO: 7) (Stefanovie et al., 2020, Nucleic Acids Res., 48:694-708). The inventors therefore next constructed Cas9 protein comprising sequences derived from all RFRDs such as BRCA2, DSS1, RAD54, and RAD52. As can be observed in FIG. 3E showing HDR directed replacement of PD1 by GFP in human T-cells, C′-Rad52 peptide fused nuclease (TG15194, SEQ ID NO: 172) had better HDR insertion efficiency (with donor DNA bW lane 14) than any other dscCas derived nuclease in that experiment (same donor DNA, lanes 1, 6, 8, 10 and 12) ostensibly due to the action of the RFRD C′-BRCA2 peptide at the site of the dsDNA break.

### Example 3

### HDR-Enhanced Chimeras

HDR enhancement may be used also to prepare specific chimeric proteins based for example on dead Cas nucleases, such as a dead or deactivated nuclease Cas (dCas) fused to a FokI-nuclease (dCas-FokI), illustrated in FIGS. 1, 2 and 4. This configuration improves gene editing specificity due to the requirement for two target sites.

The following HDR-enhanced chimeras were designed: dScCas9-FokI-ZFQ (dScCas9-FokI fused to zinc finger QQR which binds to DNA sequence GGGGAAGAA), as denoted by SEQ ID NO. 9, dScCas9-FokI-Lam (dScCas9-FokI fused to Lambda repressor DNA binding domain), as denoted by SEQ ID NO. 10, dScCas9-FokI-Strep (dScCas9-FokI fused to monomeric streptavidin), as denoted by SEQ ID NO. 11, dScCas9-FokI-Vir (dScCas9-FokI fused to monomeric virD2), as denoted by SEQ ID NO. 12, dScCas9-FokI-BRCA2 as denoted by SEQ ID NO. 13, dScCas9-FokI-DSS1 as denoted by SEQ ID NO. 14.

HDR-enhanced chimeras may include both repair factor recruitment domains and donor attachment domains, the combination of which lead to a synergistic improvement in HDR efficiency. The following HDR-enhanced chimeras were designed that included both repair factor recruitment domains and donor attachment domains: dScCas9-FokI-BRCA2-Strep (dScCas9-FokI fused to a BRCA2 peptide and to a monomeric streptavidin), as denoted by SEQ ID NO. 15, dScCas9-FokI-DSS1-Strep (dScCas9-FokI fused to a DSS1 peptide and to a monomeric streptavidin), as denoted by SEQ ID NO. 16, dScCas9-FokI-BRCA2-virD2 (dScCas9-FokI fused to a BRCA2 peptide and to a monomeric virD2), as denoted by SEQ ID NO. 17, and dScCas9-FokI-DSS1-virD2 (dScCas9-FokI fused to a DSS1 peptide and to a monomeric virD2), as denoted by SEQ ID NO. 18.

HDR-enhanced dScCas9-FokI variants were tested for activity using ddPCR on HEK293 cells. Constructs were ordered as gene synthesis constructs from various suppliers. Editing activity on human MPO gene exon 1 are shown in Table 4.

TABLE 4
Gene editing efficiencies of dScCasFok enhanced HDR constructs.

| Description | Construct | Editing % |
|---|---|---|
| dCasFok, 3NLS variant | 13661 (SEQ ID NO: 19) | 1.62 |
| dCasFok-BRCA2, 3NLS variant | 13656 (SEQ ID NO: 20) | 3.20 |
| dCasFok-DSS1, 3NLS variant | 13659 (SEQ ID NO: 21) | 8.08 |
| dCasFok-ZFQ, 1NLS variant | 13648 (SEQ ID NO: 22) | 2.35 |
| dCasFok-Strep, 1NLS variant | 13665 (SEQ ID NO: 23) | 2.17 |

Specificity conferring nucleic acids (SCNAs) targeting the human MPO gene exon 1 were encoded by plasmid GeneMsgRNA15E1. MPO gene NHEJ erroneous repair was tested in Hek293 cells, harvested 72 hours after transfection with plasmids encoding the proteins and the SCNAs. Editing efficiency was quantified by ddPCR (ddPCR #65 results are shown). NLS=Nuclear localization sequence.

Gene editing efficiencies of HDR-enhanced constructs were assessed on MPO gene exon 1, by testing levels of NHEJ-ER by ddPCR assay. Notably, chimeras comprising the RFRD, specifically, both, the dCasFok-BRCA2 and the dCasFok-DSS1 displayed enhanced activity relative to the control dCasFok (all constructs had three nuclear localization sequences so activity could be compared between them). These results demonstrate that fusions between dCasFok and HDR-enhancing domains is possible and may significantly enhance activity due to recruitment of repair proteins. dCasFok-ZFQ and dCasFok-Strep were also shown to be functional, although activity differences compared to control dCasFok may also be due to the number of NLS.

Additional HDR-enhanced constructs were designed, with different NLS, two HDR-enhancement domains, and ancestral Cas9 mutations. (Table 5). dCasFok variants with these additional HDR-enhancement domains were found to have high gene editing activity (˜20%).

TABLE 5
Gene editing efficiencies of dScCasFok enhanced HDR constructs.

| | | Editing, | Editing, |
|---|---|---|---|
| | | ddPCR | ddPCR |
| | | 76 | 81 |
| Description | Construct | (%) | (%) |
| SpCas9, 2NLS variant | 7665 (SEQ ID NO: 41) | 49.7 | 43.0 |
| dCasFok, 1NLS variant | 11241 (SEQ ID NO: 42) | 17.8 | 21.2 |
| dCasFok, 2NLS variant | 14280 (SEQ ID NO: 43) | 29.1 | 26.26 |
| dCasFok, 2NLS, 6His | 14412 (SEQ ID NO: 44) | 30.8 | 26.1 |
| dCasFok, 2NLS, N-terminal | 14287 (SEQ ID NO: 45) | 19.4 | 18.9 |
| BRCA2 | | | |
| dCasFok, 2NLS, N-terminal | 14418 (SEQ ID NO: 46) | 0.0 | 23.3 |
| BRCA2, 6His | | | |
| dCasFok, 2NLS, N-terminal | 14464 (SEQ ID NO: 47) | 25.9 | 17.7 |
| Streptavidin | | | |
| dCasFok, 2NLS, N-terminal | 14466 (SEQ ID NO: 48) | 30.1 | 23.5 |
| Streptavidin, 6His | | | |
| dCasFok, 2NLS, N-terminal | 14470 (SEQ ID NO: 49) | 24.8 | 17.5 |
| Pot | | | |
| dCasFok, 2NLS, N-terminal | 14472 (SEQ ID NO: 50) | 20.6 | 20.3 |
| Pot, 6His | | | |
| dCasFok, 1NLS, N-terminal | 14467 (SEQ ID NO: 51) | 12.1 | 10.1 |
| Streptavidin, C-terminal | | | |
| BRCA2 | | | |
| dCasFok, 1NLS, N-terminal | 14565 (SEQ ID NO: 52) | Not | 5.8 |
| Pot, C-terminal BRCA2 | | tested | |
| dCasFok, 1NLS, ancestral | 14425 (SEQ ID NO: 53) | 12.7 | 11.7 |
| RuvC + REC1/2, N- and C- | | | |
| terminal BRCA2 | | | |
| dCasFok, 2NLS, ancestral | 14324 (SEQ ID NO: 54) | 21.7 | 0.7 |
| RuvC + REC1/2, N-terminal | | | |
| BRCA2 | | | |
| dCasFok, 2NLS, ancestral | 14419 (SEQ ID NO: 55) | 16.8 | 23.1 |
| RuvC + REC1/2, N-terminal | | | |
| BRCA2, 6His | | | |

Editing efficiency was quantified by ddPCR (ddPCR #76 and 81 results are shown).
NLS = Nuclear localization sequence.

Thus, a collection of variants of dCasFok with additional domains that may enhance HDR was found to have high gene editing activity. The next aim was to evaluate HDR-based repair and how it is compared to NHEJ-erroneous repair. HDR activity may be tested using a variation of the ddPCR assay that can detect both HDR events and NHEJ-ER events (Miyaoka et al, 2018, Methods Mol Biol, 1768:349-62), or by a DNA-sequencing based assay that can distinguish HDR from NHEJ-ER (Brinkman et al, 2018, Nucleic Acid Research, 46:e58).

Additionally, the following HDR-enhanced chimeras were designed that included repair factor recruitment domains: ancestral dCas9-FokI-RAD54ntd (ancestral dCas9-FokI fused to a RAD54 N-terminal domain peptide), as denoted by SEQ ID NO. 31, and ancestral dCas9-FokI-RAD52id (ancestral dCas9-FokI fused to a RAD52 peptide that interacts with RAD51), as denoted by SEQ ID NO. 32.

Processing of DNA breaks may be affected by the molecular structure of the DNA at the break site. For example, single-stranded DNA (5′ or 3′ ends) is recognized by specific cellular factors and is processed differently than double-strand breaks. Cas9-based DNA cleavage introduces primarily blunt and 1 bp 5′ overhangs (Gisler et al., 2019, Nature Communications, 10:1598). FokI-based DNA cleavage introduces 4 bp 5′ overhangs (Smith et al., 2000, Nucleic Acids Research, 28:3361-9). Other Type IIs restriction enzymes introduce 3′ overhangs, such as MmeI (SEQ ID NO. 33), MnlI (SEQ ID NO. 34), and BfiI (SEQ ID NO. 35).

The following chimeras were designed that used nucleases that may introduce 3′ overhangs instead of 5′ overhangs: ancestral dCas9-MmeI (ancestral dCas9 fused to MmeI), as denoted by SEQ ID NO. 36, ancestral dCas9-MnlI (ancestral dCas9 fused to MnlI), as denoted by SEQ ID NO. 37, and ancestral dCas9-BfiI (ancestral dCas9 fused to BfiI), as denoted by SEQ ID NO. 38.

The following chimeras were tested (Table 6):

TABLE 6
Gene editing efficiencies of dScCasFok enhanced HDR constructs.

| | | | ddPCR116 | ddPCR116 |
|---|---|---|---|---|
| SEQ | | | Poisson | II Poisson |
| ID | TG | | correction | correction |
| NO | number | description | (old) % | % |
| 141 | 15145 | BRCA2 peptide(n-term), | 34% | 29% |
| | | ancestral dCasFok | | |
| 142 | 15146 | DSS1 peptide(n-term), | 28% | 29% |
| | | ancestral dCasFok | | |
| 143 | 15147 | BRCA2 peptide 2(n-term), | 29% | 31% |
| | | ancestral dCasFok | | |
| 144 | 15149 | RAD52 peptide(n-term), | 29% | 31% |
| | | ancestral dCasFok | | |
| 145 | 15151 | Streptavidin(n-term), ancestral | 30% | 26% |
| | | dCasFok | | |
| 146 | 15152 | BRCA2 peptide (c-term), | 31% | 31% |
| | | ancestral dCasFok | | |
| 147 | 15153 | DSS1 peptide (c-term), | 24% | 22% |
| | | ancestral dCasFok | | |
| 148 | 15154 | BRCA2 peptide 2 (c-term), | 35% | 32% |
| | | ancestral dCasFok | | |
| 149 | 15155 | RAD54 peptide (c-term), | 28% | 18% |
| | | ancestral dCasFok | | |
| 150 | 15156 | RAD52 peptide (c-term), | 32% | 32% |
| | | ancestral dCasFok | | |
| 151 | 15157 | Mdm2 peptide (c-term), | 23% | 22% |
| | | ancestral dCasFok | | |
| 152 | 15158 | Streptavidin (c-term), ancestral | 23% | 21% |
| | | dCasFok | | |
| 153 | 15166 | BRCA2 peptide(n-term), | 36% | 24% |
| | | ancestral dCasFok, his-tagged | | |
| 154 | 15167 | DSS1 peptide(n-term), | 40% | 24% |
| | | ancestral dCasFok, his-tagged | | |
| 155 | 15168 | BRCA2 peptide 2(n-term), | 26% | 23% |
| | | ancestral dCasFok, his-tagged | | |
| 156 | 15169 | RAD54 peptide(n-term), | 20% | 13% |
| | | ancestral dCasFok, his-tagged | | |
| 157 | 15170 | RAD52 peptide(n-term), | 25% | 23% |
| | | ancestral dCasFok, his-tagged | | |
| 158 | 15172 | Streptavidin(n-term), ancestral | 34% | 22% |
| | | dCasFok, his-tagged | | |
| 159 | 15176 | BRCA2 peptide (n and c-term), | 27% | 22% |
| | | ancestral dCasFok | | |
| 160 | 15177 | BRCA2 peptide (n-term), | 22% | 20% |
| | | DSS1 peptide (c-term), | | |
| | | ancestral dCasFok | | |
| 161 | 15178 | BRCA2 peptide (n-term), | 32% | 27% |
| | | BRCA2 peptide 2 (c-term), | | |
| | | ancestral dCasFok | | |
| 162 | 15180 | BRCA2 peptide (n-term), | 35% | 28% |
| | | RAD52 peptide (c-term), | | |
| | | ancestral dCasFok | | |
| 163 | 15181 | BRCA2 peptide (n-term), | 22% | 19% |
| | | Mdm2 peptide (c-term), | | |
| | | ancestral dCasFok | | |
| 164 | 15182 | BRCA2 peptide (n-term), | 23% | 15% |
| | | Streptavidin (c-term), ancestral | | |
| | | dCasFok | | |
| 165 | 15183 | DSS1 peptide(n-term), BRCA2 | 34% | 22% |
| | | peptide (c-term), ancestral | | |
| | | dCasFok | | |
| 166 | 15185 | DSS1 peptide(n-term), BRCA2 | 34% | 18% |
| | | peptide 2 (c-term), ancestral | | |
| | | dCasFok | | |
| 167 | 15186 | DSS1 peptide(n-term), RAD54 | 23% | 11% |
| | | peptide (c-term), ancestral | | |
| | | dCasFok | | |
| 168 | 15189 | DSS1 peptide(n-term), | 26% | 13% |
| | | Streptavidin (c-term), ancestral | | |
| | | dCasFok | | |
| 169 | 15190 | Streptavidin(n-term), BRCA2 | 30% | 18% |
| | | peptide (c-term), ancestral | | |
| | | dCasFok | | |
| 170 | 15191 | Streptavidin(n-term), DSS1 | 20% | 12% |
| | | peptide (c-term), ancestral | | |
| | | dCasFok | | |
| 171 | 15192 | Streptavidin(n-term), BRCA2 | 27% | 22% |
| | | peptide 2 (c-term), ancestral | | |
| | | dCasFok | | |
| 172 | 15194 | Streptavidin(n-term), RAD52 | 24% | 22% |
| | | peptide (c-term), ancestral | | |
| | | dCasFok | | |
| 41 | 7665- | Cas9 | 56% | 54% |
| | Cas9 | | | |
| 173 | 14664 | ancestral dCasFok, his-tagged | | 33% |
| 174 | 14663 | ancestral dCasFok | | 33% |
| 41 | 7665- | No guide control | 0% | 0% |
| | Cas9 | | | |
| | 7976 | | 0% | 0% |
| | (filler) | | | |

Editing efficiency was quantified by ddPCR (ddPCR #116 results are shown).

### Example 4

### Gene Editing HDR for Diabetes

Type I (juvenile) diabetes is an autoimmune disease characterized by removal of insulin-producing beta cells. Three different solutions are proposed herein: 1) sending protective gene-edited regulatory T-cells to the pancreas; 2) creating Immuno-privileged β-cells for transplantations, and 3) REST KO for ex vivo reprogramming of acinar to β cell.

### 1. Sending Protective Modified Tregs to the Pancreas

The potential therapy would involve three complementary modifications: 1) autologous naïve CD4+ cells are induced to become Treg-like cells by expression of FOXP3 (SEQ ID:58); 2) Tregs are retargeted to the beta-cells using a CAR (i.e. anti-insulin (Tenspolde et al, 2019, Journal of Autoimmunity, 103:102289), Hpi2 (Radichev et al, 2020, Cellular Immunology, 104224), or other specific targets); and 3) induce in situ proliferation/maturation/differentiation of beta-cells using Treg's membrane-bound or locally secreted proliferative factors (i.e. WNT-1 (SEQ ID NO:121), PDGF1 (SEQ ID NO:122), IGF1 (SEQ ID NO:39), TGFβ-1 (SEQ ID NO:40)). Tregs on-site will naturally immuno-protect beta-cells. Enhanced proliferation/maturation/differentiation of beta cells may support the control of type I diabetes. Allogeneic protective CAR-T is possible as well and would require removal of donor-specific HLA alleles to avoid graft-versus-host disease. This would allow a potential Universal “off-the-shelf” Treg product.

These modifications may be implemented by gene editing CD4+ cells at the constant regions of the TCRA gene (NCBI gene ID 21473) or TCRB gene (NCBI ID 21577). Guides for dCasFok targeting these regions are shown in SED ID NO:59-69 for TCRA and SEQ ID NO:70-83, for TCRB).

Homologous recombination targeting TCR constant region would be used to introduce a cassette encoding a CAR, FoxP3, and a beta cell proliferation factor. Gene editing is carried out using HDR-enhanced Cas chimeras described in Example 3. Several homologous recombination cassettes have been designed under the general framework of: CAR-FOXP3-Beta cell proliferation.

All combinations of the different factors mentioned above are used.

The following homology cassettes are delivered to the target locus by the enhanced HDR systems described in Example 3:
Left homology arm-P2A-CARanti-insulin-P2A-FOXP3-P2A-WNT1-Right homology arm.
Left homology arm-P2A-CARanti-Hpi2-FOXP3-PDGF1-Right homology arm.
Left homology arm-P2A-CAR anti-insulin-FOXP3-IGF1-Right homology arm.
Left homology arm-CAR anti-Hpi2-FOXP3-TGFB1-Right homology arm
   2. Ex-Vivo Knockout of Key Regulators Genes in β Cells or Stem Cells Transduced into β Cells in Order to Generate Hypoimmunogenic Islets

RNLS was identified as a gene whose deletion/inhibition made beta cells resistant to autoimmune killing (Cai et al Nat Metab. 2020 2:934-945). Importantly, RNLS deficiency did not affect stem cell differentiation into beta cells and RNLS knockout did not impair insulin secretion.

RNLS may be targeted by dCasFok using guides shown in SEQ ID NO:84 to 91. This would allow generation of beta cells from stem cells that are protected from immune destruction.

Another possible target for genetic knockout is IRE1α (also known as ERN1). IRE1α deletion in NOD β cells before insulitis causes their transient dedifferentiation (Lee et al 2020 Cell Metabolism 31:822-36). Dedifferentiated β cells showed diminished expression of β cell autoantigens. Knockout mice exhibit impaired T cell diabetogenic activity. IRE1α-deficient NOD mice are protected from autoimmune destruction and diabetes. Yet after short period of transient mild hyperglycemia, mice recovered and have serum insulin levels comparable with that of control non-diabetic mice.

Using dCasFok to knockout IRE1α at any step of ex-vivo β-cell differentiation may allow creation of immune-privileged β-cells for transplantations. IRE1α may be targeted by dCasFok using guides shown in SEQ ID NO:92-103.

### 3. REST KO for Ex-Vivo Reprogramming of Acinar to β Cell

The RE-1 silencing transcription factor (REST) gene is a transcription repressor that inhibits expression of endocrine genes (Elhanani et al, 2020, Cell Reports. 31:107591). Genetic knock-out of REST gene in conjunction with temporally regulated expression of the reprogramming factors under the REST promoter may allow ex-vivo reprogramming of acinar exocrine cells to endocrine β cells. Expression may be limited to the period of reprograming, dependent on the pattern of REST expression.

REST gene may be targeted by dCasFok using guides shown in SEQ ID:104-117.

Genes introduced by homologous recombination for expression under the native REST promoter include: PDX1 (SEQ ID NO:118), NGN3 (SEQ ID NO:119), MAFA (SEQ ID:120).

Still further, the PDX1-2A-NGN3-2A-MAFA construct would be delivered by the enhanced HDR chimeras described in Example 3.

While transplanting reprogrammed β cells for non-self-donors (cadaveric), an immune privileged site will be generated to protect those cells from rejection, as well as generating less immunogenicity cells (by IRE1α KO or MHC class I elimination).

### Example 5

Pure Directional Insertion, HDR Insertion without NHEJ

An experiment to test insertion via HDR of a short stretch of DNA within a genomic break in exon1 of the PD1 gene was next performed (FIG. 5A). The donor DNA used was a 99 nucleotide (nt) ssDNA with a 5′-biotin (marked/5Biosg/) for the triple purpose of a) binding to different proteins of the invention containing streptavidin as a DAD b) for protection against endogenous exonucleases and c) to avoid ligation leading to NHEJ. Additionally, phosphorothioate modifications (marked *) were made at the first two nucleotides at each end.

The Donor used herein is the 3716-PD1ex1.HDR.3 having the DNA sequence:

/5Biosg/C*A*GATCCCACAGGCGCCCTGGCCAGTCGTCTGGGCGTTT
AATTGAGTTGTCATATGTTAATAACGGTGCTACAACTGGGCTGGCGGCC
AGGATGGTT*C*T, as also denoted by SEQ ID NO: 140.

The Donor comprises two homology arms with sequences identical (sense orientation) to those flanking the expected cleavage site. The Left homology arm (LHA) consisted of 36 nt and the Right homology arm (RHA) consisted of 34 nt. The LHA matched 12 nt of the Left gRNA binding site, spanned the 15 nt gap between the guides including the expected cut site in the middle and included 9 nt of the Right gRNA binding site to the right of the cut site. The RHA matched the remaining 11 nt of the Right gRNA binding site and the sequence to the right of it. The insert was thus expected to integrate into the Right gRNA binding site. Hek293 cells in 96 wells were transfected with plasmids expressing the proteins of the invention or spCas9 as control concomitantly with a plasmid encoding the relevant guide RNAs #14082 (SEQ ID NO: 176 and 177) for proteins of the invention or #14106 (SEQ ID NO: 175), for spCas9. Construct TG numbers corresponding to Table 6 are: 15151 (SEQ ID NO: 145), 15172 (SEQ ID NO: 158), 15190 (SEQ ID NO: 169) and 15155 (SEQ ID NO: 149). Transfected plasmid DNA (total 100 ng) consisted of equivalent amounts of nuclease-expressing construct and guide expressing construct plus 100 ng donor ssDNA. Cells were collected 96 h after transfection.

PCR performed on genomic DNA was conducted as shown in FIG. 5B. Primers were pre-validated (not shown). Result (FIG. 5C) show detection only of correctly oriented insert and not of opposite orientation expected to be 50% of NHEJ insertion. This strongly indicates that all insertion was mediated directionally and thus by homology directed repair (HDR) and not by NHEJ. This result was observed in all treatments including both a nuclease and the respective guide or guide-pair but not when the guide RNA was omitted (sample A10), asserting dependence of this HDR on a programmed nuclease at the target site.

These results therefore establish the feasibility of using the chimeras of the present disclosure for effectively direct insertion of a desired nucleic acid sequence by HDR.

1-59. (canceled)

60. A nucleic acid guided genome modifier chimeric or fusion protein, complex or conjugate having enhanced homology-directed repair (HDR) comprising:
(a) at least one defective CRISPR-Cas protein (CRISPR-dCas) devoid of a nucleolytic activity or any variant or mutant thereof; and
(b) at least one nucleic acid modifier component; and at least one of:
(c) at least one donor attachment domain (DAD) for binding at least one Donor nucleic acid molecule; and
(d) at least one repair factor recruitment domain (RFRD).

61. The nucleic acid guided genome modifier chimeric protein, complex or conjugate according to claim 60, wherein at least one of:
(i) said DAD is at least one of: a sequence specific donor attachment domain, a non-sequence specific donor attachment domain and a covalent interaction domain;
(ii) said DAD is a sequence specific DAD comprising at least one of: a zinc finger DNA binding domain, a lambda repressor DNA binding domain, a Gal4 DNA binding domain and a protection of telomeres 1 protein (Pot1) ssDNA binding domain;
(iii) said DAD is a covalent interaction domain comprising a virD2 domain; and/or
(iv) said DAD is a non-sequence specific donor attachment domain comprising at least one domain of an affinity pair.

62. The nucleic acid guided genome modifier chimeric protein, complex or conjugate according to claim 60, wherein said RFRD recruits a protein involved in the HDR pathway of (double strand breaks) DSBs, optionally, wherein at least one of
(a) said protein involved in HDR is at least one of a Recombination Protein A (Rad) family member, a Fanconi Anemia Core Complex member, Tumor Suppressor p53, or C-Terminal-Binding Protein-Interacting Protein (CtIP);
(b) said RFRD comprises at least one of: the BRCA2 protein, or any fragment or peptides thereof, the DSS1 protein, or any fragment or peptides thereof, the RAD52 protein or any fragment or peptides thereof, and or the RAD54 protein, or any fragment or peptides thereof.

63. The nucleic acid guided genome modifier chimeric protein, complex or conjugate according to claim 60, wherein at least one of:
(a) said CRISPR-dCas protein at least one of Cas9, CasX, Cas12a1, CasF, Cas14a1, an ancestral Cas and Cas14b5, optionally, said CRISPR-dCas protein has reduced or abolished Protospacer Adjacent Motif (PAM) constraint, and wherein at least one of the PAM binding domain (PBD) and/or PAM recognition motif, any fragment of said PBD, and at least one amino acid residue adjacent to said PBD, of said CRISPR-dCas protein is deleted or replaced;
(b) said CRISPR-dCas protein or any variant, mutant, fusion protein, complex or conjugate thereof, is capable of binding at least one target recognition element, optionally, said at least one target recognition element is at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids or combinations thereof; and/or
(c) said at least one nucleic acid modifier component is a protein-based modifier, a nucleic acid-based modifier or any combinations thereof, and wherein said protein-based modifier is at least one of a nuclease, a methyltransferase, a methylated DNA binding factor, a transcription factor, a transcription repressor, a chromatin remodeling factor, a polymerase, a demethylase, an acetylase, a deacetylase, a kinase, a phosphatase, an integrase, a recombinase, a ligase, a topoisomerase, a girase, a helicase, and any combinations thereof.

64. The nucleic acid guided genome modifier chimeric protein, complex or conjugate according to claim 60, wherein said nucleic acid modifier component is at least one nuclease, and wherein said nuclease is a Type IIS restriction endonuclease or any fragment, variant, mutant, fusion protein or conjugate thereof, optionally, at least one of:
(i) said Type IIS restriction endonuclease is FokI or any fragment, variant, mutant, fusion protein or conjugate thereof;
(ii) said Type IIS restriction endonuclease is MmeI or any fragment, variant, mutant, fusion protein or conjugate thereof;
(iii) said Type IIS restriction endonuclease is MnlI or any fragment, variant, mutant, fusion protein or conjugate thereof;
(iv) said Type IIS restriction endonuclease is BfiI or any fragment, variant, mutant, fusion protein or conjugate thereof.

65. The nucleic acid guided genome modifier chimeric protein, complex or conjugate according to claim 64, wherein said nucleic acid guided genome modifier is a chimeric protein, said chimeric protein is any one of: dScCas9-FokI-ZFQ variant; dScCas9-FokI-Lam variant; dScCas9-FokI-Strep variant; dScCas9-FokI-Vir variant; dScCas9-FokI-BRCA2 variant; dScCas9-FokI-DSS1 variant; dScCas9-FokI-BRCA2-Strep variant; dScCas9-FokI-DSS1-Strep variant; dScCas9-FokI-BRCA2-virD2 variant; dScCas9-FokI-DSS1-virD2 variant; dCas9-BfiI variant, dCas9-MnlI variant; dCas9-MmeI variant; dCas9-FokI-RAD54ntd variant; dCasFok-BRCA2 3NLS variant; dCasFok-DSS1 3NLS variant; dCasFok-ZFQ 1NLS variant; dCasFok-Strep 1NLS variant; dCasFok, 2NLS, N-terminal BRCA2 variant; dCasFok, 2NLS, N-terminal BRCA2, 6His variant; dCasFok, 2NLS, N-terminal Streptavidin variant; dCasFok, 2NLS, N-terminal Streptavidin, 6His variant; dCasFok, 2NLS, N-terminal Pot variant; dCasFok, 2NLS, N-terminal Pot, 6His variant; dCasFok, 1NLS, N-terminal Streptavidin, C-terminal BRCA2 variant; dCasFok, 1NLS, N-terminal Pot, C-terminal BRCA2 variant; dCasFok, 1NLS, ancestral RuvC+REC1/2, N- and C-terminal BRCA2 variant; dCasFok, 2NLS variant, ancestral RuvC+REC1/2, N-terminal BRCA2 variant; dCasFok, 2NLS, ancestral RuvC+REC1/2, N-terminal BRCA2, 6His variant; ancestral dCas9-FokI-RAD52id variant; DSS1 peptide (n-term), ancestral dCasFok variant; BRCA2 peptide 2 (N-term), ancestral dCasFok variant; RAD52 peptide (n-term), ancestral dCasFok variant; Streptavidin (n-term), ancestral dCasFok variant; BRCA2 peptide (c-term), ancestral dCasFok variant; DSS1 peptide (c-term), ancestral dCasFok variant; BRCA2 peptide 2 (c-term), ancestral dCasFok variant; RAD54 peptide (c-term), ancestral dCasFok; RAD52 peptide (c-term), ancestral dCasFok variant; Mdm2 peptide (c-term), ancestral dCasFok variant; Streptavidin (c-term), ancestral dCasFok variant; BRCA2 peptide (n-term), ancestral dCasFok, his-tagged variant; DSS1 peptide (n-term), ancestral dCasFok, his-tagged variant; BRCA2 peptide 2 (n-term), ancestral dCasFok, his-tagged variant; RAD54 peptide (n-term), ancestral dCasFok, his-tagged variant; RAD52 peptide (n-term), ancestral dCasFok, his-tagged variant; Streptavidin (n-term), ancestral dCasFok, his-tagged variant; BRCA2 peptide (n and c-term), ancestral dCasFok variant; BRCA2 peptide (n-term), DSS1 peptide (c-term), ancestral dCasFok variant; BRCA2 peptide (n-term), BRCA2 peptide 2 (c-term), ancestral dCasFok variant; BRCA2 peptide (n-term), RAD52 peptide (c-term), ancestral dCasFok variant; BRCA2 peptide (n-term), Mdm2 peptide (c-term), ancestral dCasFok variants; BRCA2 peptide (n-term), Streptavidin (c-term), ancestral dCasFok variant; DSS1 peptide (n-term), BRCA2 peptide (c-term), ancestral dCasFok variant; DSS1 peptide (n-term), BRCA2 peptide 2 (c-term), ancestral dCasFok variant; DSS1 peptide (n-term), RAD54 peptide (c-term), ancestral dCasFok variant; DSS1 peptide (n-term), Streptavidin (c-term), ancestral dCasFok variant; Streptavidin (n-term), BRCA2 peptide (c-term), ancestral dCasFok variant; Streptavidin (n-term), DSS1 peptide (c-term), ancestral dCasFok variant; Streptavidin (n-term), BRCA2 peptide 2 (c-term), ancestral dCasFok variant; Streptavidin (n-term), RAD52 peptide (c-term), ancestral dCasFok variant; and ancestral dCasFok, his-tagged variant.

66. A nucleic acid molecule or any vector, construct, cassette or delivery vehicle thereof, said nucleic acid molecule comprising a nucleic acid sequence encoding at least one nucleic acid guided genome modifier chimeric protein having enhanced HDR according to claim 60, or any variant, mutant, fusion/chimeric protein, complex or conjugate thereof, wherein said nucleic acid guided genome modifier chimeric protein comprises:
(a) at least one CRISPR-dCas protein devoid of a nucleolytic activity or any variant or mutant thereof; and
(b) at least one nucleic acid modifier component; and at least one of:
(c) at least one DAD for binding at least one Donor nucleic acid molecule; and
(d) at least one RFRD.

67. A nucleic acid guided genome modifier system having enhanced HDR comprising:
(a) a nucleic acid guided genome modifier chimeric or fusion protein, complex or conjugate having enhanced homology-directed repair (HDR), according to claim 60, or at least one nucleic acid sequence encoding said nucleic acid guided genome modifier chimeric or fusion protein, wherein said nucleic acid guided genome modifier chimeric or fusion protein comprises:
(i) at least one CRISPR-dCas protein devoid of a nucleolytic activity or any variant or mutant thereof; and
(ii) at least one nucleic acid modifier component; and at least one of:
(iii) at least one DAD for binding at least one Donor nucleic acid molecule; and
(iv) at least one RFRD;
said system further comprises at least one of:
(b) at least one donor nucleic acid molecule; and
(c) at least one target recognition element, or any nucleic acid sequence encoding said target recognition element.

68. The system according to claim 67, wherein at least one of:
(a) said DAD is at least one of: a sequence specific donor attachment domain, a non-sequence specific donor attachment domain and a covalent interaction domain;
(b) said DAD is a sequence specific DAD comprising at least one of a zinc finger DNA binding domain, a lambda repressor DNA binding domain, a Gal4 DNA binding domain and a Pot1 ssDNA binding domain;
(c) said DAD is a covalent interaction domain comprising a virD2 domain; and/or
(d) said DAD is a non-sequence specific donor attachment domain comprising at least one domain of an affinity pair.

69. The system according to claim 67, wherein at least one of:
(a) said RFRD recruits a protein involved in the HDR pathway of DSBs;
(b) said protein involved in HDR is any one of a Rad family member, a Fanconi Anemia Core Complex member, Tumor Suppressor p53, or CtIP;
(c) said repair factor recruitment domain RFRD comprises at least one of: the BRCA2 protein, or any fragment or peptides thereof; the DSS1 protein, or any fragment or peptides thereof, the RAD52 protein, or any fragment or peptides thereof or the RAD54 protein, any fragment or peptides thereto.

70. The system according to claim 67, wherein at least one of:
(a) said CRISPR-dCas protein comprises at least one of Cas9, CasX, Cas12a1, CasF, Cas14a1, an ancestral Cas and Cas14b5, optionally, said CRISPR-dCas protein has reduced or abolished PAM constraint, and wherein at least one of the PDB of said CRISPR-dCas protein, any fragment of said PBD, and at least one amino acid residue adjacent to said PBD, is deleted or replaced; and/or
(b) said CRISPR-dCas protein, is capable of binding at least one target recognition element, and wherein said at least one target recognition element is at least one nucleic acid target recognition element being at least one of: a single strand RNA molecule, a double strand RNA molecule, a single strand DNA, a double strand DNA, a modified DNA molecule, a modified RNA molecule, a LNA, a PNA and any hybrid or combinations thereof.

71. The system according to claim 67, wherein said donor nucleic acid molecule comprises at least one nucleic acid sequence for incorporation into a target site within a target nucleic acid sequence, said donor nucleic acid molecule is flanked by at least one homology arm, optionally, at least one of:
(a) said donor nucleic acid molecule further comprises an attachment region that binds said DAD of said nucleic acid guided genome modifier chimeric protein, complex or conjugate;
(b) said at least one nucleic acid sequence for incorporation of said donor nucleic acid molecule, is a replacement sequence of a target nucleic acid of interest in said target site.

72. At least one cell or population of cells, said cell comprising and/or modified by at least one of:
(a) a nucleic acid guided genome modifier chimeric or fusion protein, complex or conjugate having enhanced HDR according to claim 60, or at least one nucleic acid sequence encoding said nucleic acid guided genome modifier chimeric or fusion protein, wherein said nucleic acid guided genome modifier chimeric or fusion protein comprises:
(i) at least one CRISPR-dCas protein devoid of a nucleolytic activity or any variant or mutant thereof; and
(ii) at least one nucleic acid modifier component; and at least one of:
(iii) at least one DAD for binding at least one Donor nucleic acid molecule; and
(iv) at least one RFRD;
(b) at least one donor nucleic acid molecule;
(c) at least one target recognition element or any nucleic acid sequence encoding said target recognition element;
(d) at least one nucleic acid cassette or any vector or vehicle comprising at least one of; (i) the nucleic acid sequence of (a); (ii) the nucleic acid sequence of (b); (iii) the nucleic acid sequence of (c); and (iv) the nucleic acid sequence of: (a) and at least one of (b) and (c); and
(e) at least one system comprising (a) and at least one of (b) and (c).

73. The cell according to claim 72, wherein said CRISPR-dCas protein comprises at least one of Cas9, CasX, Cas12a1, CasF, Cas14a1, an ancestral Cas and Cas14b5, optionally, said CRISPR-dCas protein has reduced or abolished PAM constraint, and wherein at least one of the PBD of said CRISPR-dCas, any fragment of said PBD, and at least one ammo acid residue adjacent to said PBD, is deleted or replaced.

74. A composition comprising at least one of:
(a) a nucleic acid guided genome modifier chimeric or fusion protein, complex or conjugate having enhanced HDR, according to claim 60, or at least one nucleic acid sequence encoding said nucleic acid guided genome modifier chimeric or fusion protein, wherein said nucleic acid guided genome modifier chimeric or fusion protein comprises:
(i) at least one CRISPR-dCas protein devoid of a nucleolytic activity or any variant or mutant thereof; and
(ii) at least one nucleic acid modifier component; and at least one of:
(iii) at least one DAD for binding at least one Donor nucleic acid molecule; and
(iv) at least one RFRD;
(b) at least one donor nucleic acid molecule;
(c) at least one target recognition element or any nucleic acid sequence encoding said target recognition element;
(d) at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c);
(e) at least one system comprising (a) and at least one of (b) and (c); and
(f) at least one cell comprising and/or modified by at least one of: the nucleic acid cassette or any vector or vehicle of (d) and the at least one system of (e); or any matrix, nano- or micro-particle comprising at least one of (a), (b), (c), (d), (e) and (f), or a cell population comprising said cells, said composition optionally further comprises at least one of pharmaceutically acceptable carrier/s, diluent/s, excipient/s and additive/s.

75. The composition according to claim 74, wherein said CRISPR-dCas protein comprises at least one of Cas9, CasX, Cas12a1, CasF, Cas14a1, an ancestral Cas and Cas14b5, optionally, said CRISPR-dCas protein has reduced or abolished PAM constraint, and wherein at least one of the PBD of said CRISPR-dCas protein, any fragment of said PBD, and at least one amino acid residue adjacent to said PBD, is deleted or replaced.

76. A method of modifying at least one target nucleic acid sequence of interest in at least one cell, said method comprising the steps of contacting said cell with at least one of:
(a) a nucleic acid guided genome modifier chimeric or fusion protein, complex or conjugate having enhanced HDR, or at least one nucleic acid sequence encoding said nucleic acid guided genome modifier chimeric or fusion protein, wherein said nucleic acid guided genome modifier chimeric or fusion protein comprises:
(i) at least one CRISPR-dCas protein devoid of a nucleolytic activity or any variant or mutant thereof; and
(ii) at least one nucleic acid modifier component; and at least one of:
(iii) at least one DAD for binding at least one Donor nucleic acid molecule; and
(iv) at least one RFRD;
(b) at least one donor nucleic acid molecule, said donor molecule comprises at least one replacement sequence for incorporation in, and/or for replacing, the target nucleic acid sequence of interest;
(c) at least one target recognition element or any nucleic acid sequence encoding said target recognition element; said target recognition element specifically recognizes and binds said target sequence;
(d) at least one nucleic acid cassette or any vector or vehicle comprising at least one of: (i) the nucleic acid sequence of (a); (ii) the nucleic acid sequence of (b); (iii) the nucleic acid sequence of (c); and (iv) the nucleic acid sequence of (a) and at least one of (b) and (c); and
(e) at least one system or composition comprising (a) and at least one of (b) and (c).

77. The method of claim 76, wherein at least one of:
(a) said CRISPR-dCas protein comprises at least one of Cas9, CasX, Cas12a1, CasF, Cas14a1, an ancestral Cas and Cas14b5, optionally, said CRISPR-dCas protein has reduced or abolished PAM constraint, and wherein at least one of the PBD of said CRISPR-dCas, any fragment of said PBD, and at least one amino acid residue adjacent to said PBD, is deleted or replaced;
(b) said cell is of at least one organism of the biological kingdom Animalia;
(c) said target nucleic acid sequence of interest is at least one of: at least one gene encoding at least one tumor associated antigen (TAA), at least one gene encoding a protein involved in at least one metabolic disorder, at least one gene encoding a protein involved in at least one congenital disorder, at least one gene encoding receptors for at least one viral antigen, at least one gene associated with at least one inborn error of metabolism (IEM) disorder, Immunoglobulin locus, T cell receptor (TCR) locus, safe harbor site/s (SHS), and any coding sequence or non-coding sequence involved with at least one pathologic disorder; and/or
(d) said cell is of at least one organism of the biological kingdom Plantae.

78. A method of curing or treating, preventing, inhibiting, reducing, eliminating, protecting or delaying the onset of a pathologic disorder or condition in a subject in need thereof, said method comprising the steps of administering to said subject an effective amount of at least one of:
(a) a nucleic acid guided genome modifier chimeric or fusion protein, complex or conjugate having enhanced HDR, or at least one nucleic acid sequence encoding said nucleic acid guided genome modifier chimeric or fusion protein, wherein said nucleic acid guided genome modifier chimeric or fusion protein comprises:
(i) at least one CRISPR-dCas protein devoid of a nucleolytic activity or any variant or mutant thereof; and
(ii) at least one nucleic acid modifier component; and at least one of:
(iii) at least one DAD for binding at least one Donor nucleic acid molecule; and
(iv) at least one RFRD;
(b) at least one donor nucleic acid molecule, said donor molecule comprises at least one replacement sequence for incorporation in, and/or for replacing, a target nucleic acid sequence of interest in the genome of the treated subject;
(c) at least one target recognition element, or any nucleic acid sequence encoding said target recognition element, the target recognition element specifically recognizes and binds the target sequence in the genome of at least one cell of the treated subject;
(d) at least one nucleic acid cassette or any vector or vehicle comprising at least one of; (i) the nucleic acid sequence of (a); (i) the nucleic acid sequence of (b); (iii) the nucleic acid sequence of (c); and (iv) the nucleic acid sequence of (a) and at least one of (b) and (c);
(e) at least one system comprising (a) and at least one of (b) and (c);
(f) at least one cell comprising and/or modified by at least one of: (a), (b), (c), (d) and (e); and
(g) at least one composition comprising at least one of (a), (b), (c), (d), (e) and (f).

79. The method according to claim 78, wherein at least one of:
(a) said CRISPR-dCas protein comprises at least one of Cas9, CasX, Cas12a1, CasF, Cas14a1, an ancestral Cas and Cas14b5, optionally, said CRISPR-dCas protein has reduced or abolished PAM constraint, and wherein at least one of the PBD of said CRISPR-dCas protein, any fragment of said PBD, and at least one amino acid residue adjacent to said PBD, is deleted or replaced;
(b) said subject is of the biological kingdom Animalia or of the biological kingdom Plantae; and/or
(c) said subject is of the biological kingdom Animalia, and wherein said subject is a mammalian subject, optionally, at least one of:
(i) said pathologic disorder is any one of a proliferative disorder, a metabolic disorder, a congenital disorder, an immune-related condition, an inflammatory condition, a disorder caused by a pathogen, an autoimmune disorder and an IEM disorder; and/or
(ii) said method comprises the step of administering to said subject a therapeutically effective amount of at least one of said cell, or of any composition comprising said at least one cell, wherein said at least one cell is of an autologous or allogeneic source.
